SEQUENCE LISTING (57 sequences)

SEQ ID NO: 1 - DNA, 912 bp, Mus musculus
atggtgaagttcctgctaattgcgattgcattaggtgtatcctgtgcacatcatgaatctcttgatatcagtccctcagaggttaatggggactggcacaccctttacatagctgcagacaaggtggagaaagtaaagatgaatggagacctgagagcgtactttgagcatatggagtgcaatgacgactgtgggacactcaaagtcaaattccatgtccagatgaatggcaagtgtcagacacacactgttgtgggagaaaaacaagaagatgggcggtacactactgactgtgagtataaattcgaagttgtaatgaaggaagatggcgcccttttctttcacaacgttaatgtggatgagagcggacaggagacaaatgtgattttagttgctggaaaaggagagaccctgagcaaagcacagaagcaggagcttgggaagctggtcaaggaatacaatattccaaaggagaatatccagcacttggcacccacaggttttaaaactgttgtactcatctgggcactgcagacagatgggccatggaaaactatagctatcgctgctgataatgtagacaaaatagagattagtggagaggacaaaatagagattagtggagagctgaggctctattttcatcaaattacttgtgaaaaggaatgcaagaaaatgaatgtcacattttatgtcaatgaaaatggacaatgttcattgacaacaatcactgggtatttgcaagatgatggcaacacctacagatcccaatttcaaggggataatcattatgcaactgtgaggacgacaccagagaacatagtattttatagtgagaatgtggacagagctggccggaaaacaaaattggtatatgttgttggtaagaatggcagtggatctctgaaatag

SEQ ID NO: 2 - protein, 303 aa, Mus musculus
MVKFLLIAIALGVSCAHHESLDISPSEVNGDWHTLYIAADKVEKVKMNGDLRAYFEHMECNDDCGTLKVKFHVQMNGKCQTHTVVGEKQEDGRYTTDCEYKFEVVMKEDGALFFHNVNVDESGQETNVILVAGKGETLSKAQKQELGKLVKEYNIPKENIQHLAPTGFKTVVLIWALQTDGPWKTIAIAADNVDKIEISGEDKIEISGELRLYFHQITCEKECKKMNVTFYVNENGQCSLTTITGYLQDDGNTYRSQFQGDNHYATVRTTPENIVFYSENVDRAGRKTKLVYVVGKNGSGSLK

SEQ ID NO: 3 - DNA, 758 bp, Rattus norvegicus
gaatccaggctctaacatggtgaagtttctgctgattgttcttgcattaggtgtatcctgtgcacatcatgaaaatcttgatatcagtccctcagaggttaatggggactggcgcaccctttacatagttgcagataatgtggagaaggtagcagaaggtggatccctgagagcttactttcagcacatggaatgtggtgatgaatgccaggaactcaaaatcatattcaatgtcaagttggacagtgaatgtcagacacacactgttgtgggacaaaaacatgaagatgggcggtacactactgactactctggtagaaattacttccatgttttgaagaagacagatgacattattttctttcacaacgttaatgtcgatgagagtggaaggagacaatgtgatttagttgctgggaaaagagaggacctgaacaaagcacagaagcaggagcttaggaagctggctgaggagtataatattccaaatgagaatacccagcacttggtgcccacagacacttgtaaccaataaagactccatatggcttcacaaaggacagcaaggtcagcaatatttcccacatcaccttttccatgaaatcagaatcgtgacaatgaagataactcatccttttcttattttttcttttcatctttcctatgaagccagaaaatctgcttcgtggatttgtttcccaccctcctatcatggtactgattcttctgttgataaaataaatttatttttcatgcac

SEQ ID NO: 4 - DNA, 732 bp, Rattus norvegicus
acacacttccagggtgagctgccttgtgtgagagcccagtgactggagatgaagagccggctcctcaccgtcctgctgctggggctgatggctgtcctgaaggctcaggaagccccacctgatgaccaggaggatttctctgggaagtggtacacaaaggccacggtttgtgacaggaaccacacagatgggaagagacctatgaaagtgttccctatgactgtgacagccctggaaggaggggacttagaggtccggataacattccgggggaagggtcattgtcatttgagacgaattacgatgcacaaaactgatgagcctggcaagtacactaccttcaaaggcaagaagaccttctatactaaggagattcctgtaaaggaccactacatcttctacattaaaggccagcgccatgggaaatcatatctgaaggggaaactcgtggggagagactctaaggacaacccagaggccatggaggaattcaagaaatttgtaaagagcaagggattcagagaagaaaacattactgtccctgagctgttggatgagtgtgtacctgggagtgactaggcacagctgcccgtcaggatagagttgctgatcctgccctaatgctgactcagttctgatacatcctgggagctcccgaactccagacgactttcctcaccttcatggatggacttcccttccacctcagcttcacccaccccagcacagctt

SEQ ID NO: 5 - DNA, 1003 bp, Rattus norvegicus
tgggcaccatcagcagagagattgtcccgacagagaggcaattctattccctaccaacatgaagctgttgctgctgctgctgtgtctgggcctgaccctggtctgtggccatgcagaagaagctagtttcgagagagggaacctcgatgtggacaagctcaatggggattggttttctattgtcgtggcctctgataaaagagaaaagatagaagagaacggcagcatgagagtttttgtgcagcacatcgatgtcttggagaattccttaggcttcacgttccgtattaaggaaaatggagtgtgcacagaattttctttggttgccgacaaaacagcaaaggatggcgaatattttgttgagtatgacggagaaaatacatttactatactgaagacagactatgacaattatgtcatgtttcatctcgttaatgtcaacaacggggaaacattccagctgatggagctctacggcagaacaaaggatctgagttcagacatcaaggaaaagtttgcaaaactatgtgtggcacatggaatcactagggacaatatcattgacctaaccaagactgatcgctgtctccaggcccgaggttgaagaaaggcctgagcctccagattgcagggcaagatctatttcttcatcctttgttctatacaatagagtgcctctctgtccagaagtcaatccaagaagtgcttaatgggttcctttattctttcttcctggattactccgtgctgagtggagacttctcaccaggactccagcattaccatttcctgtccatggagcatcctgagacaaattctgcgatctgatttccatcctgtctcacagaaaagtgcaatcctggtctctccagcatcttccctagttacccaggacaacacatcgagaattaaaagctttcttaaatttctctttgccccactcatgatcattccgcacaaatttcttgctcttgcagtgcaataaatgattacccttgcactt

SEQ ID NO: 6 - protein, 172 aa, Rattus norvegicus
MVKFLLIVLALGVSCAHHENLDISPSEVNGDWRTLYIVADNVEKVAEGGSLRAYFQHMECGDECQELKIIFNVKLDSECQTHTVVGQKHEDGRYTTDYSGRNYFHVLKKTDDIIFFHNVNVDESGRRQCDLVAGKREDLNKAQKQELRKLAEEYNIPNENTQHLVPTDTCNQ

SEQ ID NO: 7 - protein, 176 aa, Rattus norvegicus
MKSRLLTVLLLGLMAVLKAQEAPPDDQEDFSGKWYTKATVCDRNHTDGKRPMKVFPMTVTALEGGDLEVRITFRGKGHCHLRRITMHKTDEPGKYTTFKGKKTFYTKEIPVKDHYIFYIKGQRHGKSYLKGKLVGRDSKDNPEAMEEFKKFVKSKGFREENITVPELLDECVPGSD

SEQ ID NO: 8 - protein, 181 aa, Rattus norvegicus
MKLLLLLLCLGLTLVCGHAEEASFERGNLDVDKLNGDWFSIVVASDKREKIEENGSMRVFVQHIDVLENSLGFTFRIKENGVCTEFSLVADKTAKDGEYFVEYDGENTFTILKTDYDNYVMFHLVNVNNGETFQLMELYGRTKDLSSDIKEKFAKLCVAHGITRDNIIDLTKTDRCLQARG

SEQ ID NO: 9 - DNA, 741 bp, Homo sapiens
cgcccagtgacctgccgaggtcggcagcacagagctctggagatgaagaccctgttcctgggtgtcacgctcggcctggccgctgccctgtccttcaccctggaggaggaggatatcacagggacctggtacgtgaaggccatggtggtcgataaggactttccggaggacaggaggcccaggaaggtgtccccagtgaaggtgacagccctgggcggtgggaacttggaagccacgttcaccttcatgagggaggatcggtgcatccagaagaaaatcctgatgcggaagacggaggagcctggcaaattcagcgcctatgggggcaggaagctcatatacctgcaggagctgcccgggacggacgactacgtcttttactgcaaagaccagcgccgtgggggcctgcgctacatgggaaagcttgtggcatctgctccctgcagggccgtgccgctgtccccacgtcggctcacctggccacctcacctgcaggtaggaatcctaataccaacctggaggccctggaagaatttaagaaattggtgcagcacaagggactctcggaggaggacattttcatgcccctgcagacgggaagctgcgttctcgaacactaggcagcccccgggtctgcacctccagagcccaccctaccaccagacacagagcccggaccacctggacctaccctccagccatgacccttccctgctcccacccacctgactccaaataaag

SEQ ID NO: 10 - DNA, 782 bp, Homo sapiens
cgcccagtgacctgccgaggtcggcagcacagagctctggagatgaagaccctgttcctgggtgtcacgctcggcctggccgctgccctgtccttcaccctggaggaggaggatatcacagggacctggtacgtgaaggccatggtggtcgataaggactttccggaggacaggaggcccaggaaggtgtccccagtgaaggtgacagccctgggcggtgggaagttggaagccacgttcaccttcatgagggaggatcggtgcatccagaagaaaatcctgatgcggaagacggaggagcctggcaaatacagcgcctgcttgtccgcagtcgagatggaccagatcacgcctgccctctgggaggccctagccattgacacattgaggaagctgaggattgggacaaggaggccaaggattagatgggggcaggaagctcatgtacctgcaggagctgcccaggagggaccactacatcttttactgcaaagaccagcaccatgggggcctgctccacatgggaaagcttgtgggtaggaattctgataccaaccgggaggccctggaagaatttaagaaattggtgcagcgcaagggactctcggaggaggacattttcacgcccctgcagacgggaagctgcgttcccgaacactaggcagcccccgggtctgcacctccagagcccaccctaccaccagacacagagcccggaccacctggacctaccctccagccatgacccttccctgctcccacccacctgactccaaataaag

SEQ ID NO: 11 - DNA, 590 bp, Homo sapiens
cgaggtcggcagcacagagctctggagatgaagaccctgttcctgggtgtcacgctcggcctggccgctgccctgtccttcaccctggaggaggaggatatcacagggacctggtacgtgaaggccatggtggtcgataaggactttccggaggacaggaggcccaggaaggtgtccccagtgaaggtgacagccctgggcggtgggaacttggaagccacgttcaccttcatgagggaggatcggtgcatccagaagaaaatcctgatgcggaagacggaggagcctggcaaattcagcgcctatgggggcaggaagctcatatacctgcaggagctgcccgggacggacgactacgtcttttactgcaaagaccagcgccgtgggggcctgcgctacatgggaaagcttgtgggtaggaatcctaataccaacctggaggccctggaagaatttaagaaattggtgcagcacaagggactctcggaggaggacattttcatgcccctgcagacgggaagctgcgttctcgaacactaggcagcccccgggtctgcacctccagagcccaccctaccaccagacacaga

SEQ ID NO: 12 - protein, 228 aa, Homo sapiens
MKTLFLGVTLGLAAALSFTLEEEDITGTWYVKAMVVDKDFPEDRRPRKVSPVKVTALGGGNLEATFTFMREDRCIQKKILMRKTEEPGKFSAYGGRKLIYLQELPGTDDYVFYCKDQRRGGLRYMGKLVASAPCRAVPLSPRRLTWPPHLQVGILIPTWRPWKNLRNWCSTRDSRRRTFSCPCRREAAFSNTRQPPGLHLQSPPYHQTQSPDHLDLPSSHDPSLLPPT

SEQ ID NO: 13 - protein, 165 aa, Homo sapiens
MKTLFLGVTLGLAAALSFTLEEEDITGTWYVKAMVVDKDFPEDRRPRKVSPVKVTALGGGKLEATFTFMREDRCIQKKILMRKTEEPGKYSACLSAVEMDQITPALWEALAIDTLRKLRIGTRRPRIRWGQEAHVPAGAAQEGPLHLLLQRPAPWGPAPHGKACG

SEQ ID NO: 14 - protein, 170 aa, Homo sapiens
MKTLFLGVTLGLAAALSFTLEEEDITGTWYVKAMVVDKDFPEDRRPRKVSPVKVTALGGGNLEATFTFMREDRCIQKKILMRKTEEPGKFSAYGGRKLIYLQELPGTDDYVFYCKDQRRGGLRYMGKLVGRNPNTNLEALEEFKKLVQHKGLSEEDIFMPLQTGSCVLEH

SEQ ID NO: 15 - protein, 159 aa, Bos taurus
AQEEEAEQNLSELSGPWRTVYIGSTNPEKIQENGPFRTYFRELVFDDEKGTVDFYFSVKRDGKWKNVHVKATKQDDGTYVADYEGQNVFKIVSLSRTHLVAHNINVDKHGQTTELTELFVKLNVEDEDLEKFWKLTEDKGIDKKNVVNFLENEDHPHPE

SEQ ID NO: 16 - DNA, 522 bp, Sus scrofa
atgaagagtctgctgctgagtctggtccttggtctggtttgtgcccaggaacctcaacctgaacaagatccctttgagctttcaggaaaatggataaccagctacataggctctagtgacctggagaagattggagaaaatgcacccttccaggttttcatgcgtagcattgaatttgatgacaaagagagcaaagtatacttgaacttttttagcaaggaaaatggaatctgtgaagaattttcgctgatcggaaccaaacaagaaggcaatacttacgatgttaactacgcaggtaacaacaaatttgtagttagttatgcgtccgaaactgccctgataatctctaacatcaatgtggatgaagaaggcgacaaaaccataatgacgggactgttgggcaaaggaactgacattgaagaccaagatttggagaagtttaaagaggtgacaagagagaacgggattccagaagaaaatattgtgaacatcatcgaaagagatgactgtcctgccaagtga

SEQ ID NO: 17 - protein, 173 aa, Sus scrofa
MKSLLLSLVLGLVCAQEPQPEQDPFELSGKWITSYIGSSDLEKIGENAPFQVFMRSIEFDDKESKVYLNFFSKENGICEEFSLIGTKQEGNTYDVNYAGNNKFVVSYASETALIISNINVDEEGDKTIMTGLLGKGTDIEDQDLEKFKEVTRENGIPEENIVNIIERDDCPAK

SEQ ID NO: 18 - DNA, 1824 bp, Bos taurus
atgaagtgggtgacttttatttctcttctccttctcttcagctctgcttattccaggggtgtgtttcgtcgagatacacacaagagtgagattgctcatcggtttaaagatttgggagaagaacattttaaaggcctggtactgattgccttttctcagtatctccagcagtgtccatttgatgagcatgtaaaattagtgaacgaactaactgagtttgcaaaaacatgtgttgctgatgagtcccatgccggctgtgaaaagtcacttcacactctctttggagatgaattgtgtaaagttgcatcccttcgtgaaacctatggtgacatggctgactgctgtgagaaacaagagcctgaaagaaatgaatgcttcctgagccacaaagatgatagcccagacctccctaaattgaaaccagaccccaatactttgtgtgatgagtttaaggcagatgaaaagaagttttggggaaaatacctatacgaaattgctagaagacatccctacttttatgcaccagaactcctttactatgctaataaatataatggagtttttcaagaatgctgccaagctgaagataaaggtgcctgcctgctaccaaagattgaaactatgagagaaaaagtactgacttcatctgccagacagagactcaggtgtgccagtattcaaaaatttggagaaagagctttaaaagcatggtcagtagctcgcctgagccagaaatttcccaaggctgagtttgtagaagttaccaagctagtgacagatctcacaaaagtccacaaggaatgctgccatggtgacctacttgaatgcgcagatgacagggcagatcttgccaagtacatatgtgataatcaagatacaatctccagtaaactgaaggaatgctgtgataagcctttgttggaaaaatcccactgcattgctgaggtagaaaaagatgccatacctgaaaacctgcccccattaactgctgactttgctgaagataaggatgtttgcaaaaactatcaggaagcaaaagatgccttcctgggctcgtttttgtatgaatattcaagaaggcatcctgaatatgctgtctcagtgctattgagacttgccaaggaatatgaagccacactggaggaatgctgtgccaaagatgatccacatgcatgctattccacagtgtttgacaaacttaagcatcttgtggatgagcctcagaatttaattaaacaaaactgtgaccaattcgaaaaacttggagagtatggattccaaaatgcgctcatagttcgttacaccaggaaagtaccccaagtgtcaactccaactctcgtggaggtttcaagaagcctaggaaaagtgggtactaggtgttgtacaaagccggaatcagaaagaatgccctgtactgaagactatctgagcttgatcctgaaccggttgtgcgtgctgcatgagaagacaccagtgagtgaaaaagtcaccaagtgctgcacagagtcattggtgaacagacggccatgtttctctgctctgacacctgatgaaacatatgtacccaaagcctttgatgagaaattgttcaccttccatgcagatatatgcacacttcccgatactgagaaacaaatcaagaaacaaactgcacttgttgagctgttgaaacacaagcccaaggcaacagaggaacaactgaaaaccgtcatggagaattttgtggcttttgtagacaagtgctgtgcagctgatgacaaagaagcctgctttgctgtggagggtccaaaacttgttgtttcaactcaaacagccttagcctaa

SEQ ID NO: 19 - protein, 607 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLTSSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 20 - DNA, 2215 bp, Homo sapiens
agcttttctcttctgtcaaccccacacgcctttggcacaatgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataacatctacatttaaaagcatctcagcctaccatgagaataagagaaagaaaatgaagatcaaaagcttattcatctgttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttctgtgggctaattaaataaatcactaatactcttctaagtt

SEQ ID NO: 21 - protein, 609 aa, Homo sapiens [sequence omitted]

SEQ ID NO: 22 - DNA, 2051 bp, Sus scrofa
accttttctcttctatcaaccccacaagcctttggcacaatgaagtgggtgacttttatttcccttctctttctcttcagctctgcttattccaggggtgtgtttcgtcgagatacatacaagagtgaaattgctcatcggtttaaagatttgggagaacaatatttcaaaggcctagtgctgattgccttttctcagcatctccagcaatgcccatatgaagagcatgtgaaattagtgagggaagtaactgagtttgcaaaaacatgtgttgctgatgagtcagctgaaaattgtgacaagtcaattcacactctctttggagataaattatgtgcaattccatcccttcgtgaacactatggtgacttggctgactgctgtgaaaaagaagagcctgagagaaacgaatgcttcctccaacacaaaaatgataaccccgacatccctaaattgaaaccagaccctgttgctttatgcgctgacttccaggaagatgaacagaagttttggggaaaatacctatatgaaattgccagaagacatccctatttctacgccccagaactcctttattatgccattatatataaagatgttttttcagaatgctgccaagctgctgataaagctgcctgcctgttaccaaagattgagcatctgagagaaaaagtactgacttccgccgccaaacagagacttaagtgtgccagtatccaaaaattcggagagagagctttcaaagcatggtcattagctcgcctgagccagagatttcccaaggctgactttacagagatttccaagatagtgacagatcttgcaaaagtccacaaggaatgctgccatggtgacctgcttgaatgtgcagatgacagggcggatcttgccaaatatatatgtgaaaatcaagacacaatctccactaaactgaaggaatgctgtgataagcctctgttggaaaaatcccactgcattgctgaggcaaaaagagatgaattgcctgcagacctgaacccattagaacatgattttgttgaagataaggaagtttgtaaaaactataaagaagcaaagcatgtcttcctgggcacgtttttgtatgagtattcaagaaggcacccagactactctgtctcattgctgctgagaattgccaagatatatgaagccacactggaggactgctgtgccaaagaggatcctccggcatgctatgccacagtgtttgataaatttcagcctcttgtggatgagcctaagaatttaatcaaacaaaactgtgaactttttgaaaaacttggagagtatggattccaaaatgcgctcatagttcgttacaccaagaaagtaccccaagtgtcaactccaactcttgtggaggtcgcaagaaaactaggactagtgggctctaggtgttgtaagcgtcctgaagaagaaagactgtcctgtgctgaagactatctgtccctggtcctgaaccggttgtgcgtgttgcacgagaagacaccagtgagcgaaaaagttaccaaatgctgcacagagtccttggtgaacagacggccttgcttttctgctctgacaccagacgaaacatacaaacccaaagaatttgttgagggaaccttcaccttccatgcagacctatgcacacttcctgaggatgagaaacaaatcaagaagcaaactgcactcgttgagttgttgaaacacaagcctcatgcaacagaggaacaactgagaactgtcctgggcaactttgcagcctttgtacaaaagtgctgcgccgctcctgaccatgaggcctgctttgctgtggagggtccgaaatttgttattgaaattcgagggatcttagcctaaacaacacagtgacaagcatctcagactaccctgagaataagagaaagagaaatgaagacctagacttatccatctctttttcttttctgttggttttaaaccaacaccctgtctaaagtacacaaatttctttaaatattttgcctcttttctctgtgctacaattaataaaaaaatgaaaagaatct

SEQ ID NO: 23 - protein, 607 aa, Sus scrofa
MKWVTFISLLFLFSSAYSRGVFRRDTYKSEIAHRFKDLGEQYFKGLVLIAFSQHLQQCPYEEHVKLVREVTEFAKTCVADESAENCDKSIHTLFGDKLCAIPSLREHYGDLADCCEKEEPERNECFLQHKNDNPDIPKLKPDPVALCADFQEDEQKFWGKYLYEIARRHPYFYAPELLYYAIIYKDVFSECCQAADKAACLLPKIEHLREKVLTSAAKQRLKCASIQKFGERAFKAWSLARLSQRFPKADFTEISKIVTDLAKVHKECCHGDLLECADDRADLAKYICENQDTISTKLKECCDKPLLEKSHCIAEAKRDELPADLNPLEHDFVEDKEVCKNYKEAKHVFLGTFLYEYSRRHPDYSVSLLLRIAKIYEATLEDCCAKEDPPACYATVFDKFQPLVDEPKNLIKQNCELFEKLGEYGFQNALIVRYTKKVPQVSTPTLVEVARKLGLVGSRCCKRPEEERLSCAEDYLSLVLNRLCVLHEKTPVSEKVTKCCTESLVNRRPCFSALTPDETYKPKEFVEGTFTFHADLCTLPEDEKQIKKQTALVELLKHKPHATEEQLRTVLGNFAAFVQKCCAAPDHEACFAVEGPKFVIEIRGILA

SEQ ID NO: 24 - DNA, 1996 bp, Oryctolagus cuniculus
attcaatataaagaagggtttggacatctttctcctactggtaccacggaattttggcacaatgaagtgggtaacctttatctcccttcttttcctcttcagctctgcttattccaggggtgtgtttcgccgagaagcacataaaagtgagattgctcatcggtttaatgatgtgggagaagaacatttcataggcctggtgctgattaccttttctcagtatctccagaagtgcccatatgaagagcatgcgaagttagtgaaggaagtaacagacttggcaaaagcatgtgttgctgatgagtcagcagcaaattgtgacaaatcacttcatgatatttttggagacaaaatctgtgcattgccaagtcttcgtgacacctatggtgacgtggctgactgctgtgagaaaaaagaacctgagcgaaacgaatgcttcctgcaccacaaggatgataaacccgacttgcctccgtttgcgagaccagaagctgatgttttgtgcaaagcctttcatgatgatgaaaaggcattctttggacactatttatatgaagttgccagaagacatccttacttttatgcccctgaactcctttactatgctcagaagtacaaagccattctaacagaatgttgcgaagctgctgataaaggggcctgcctcacacctaagcttgatgctttggaaggaaaaagcctgatttcagctgcccaagagagactcaggtgtgccagtattcagaaatttggagacagagcttacaaagcatgggcacttgttcgtctgagccaaagatttcccaaggctgacttcacagacatttccaagatagtgacagatctcaccaaagtccacaaggaatgctgccacggtgacctgcttgaatgtgcagatgacagggcggaccttgccaagtacatgtgtgaacatcaggaaacaatctccagtcatctgaaggaatgctgtgataagccaatattggaaaaagcccactgcatttatggtttgcataatgatgagacacctgctggcttgccagcagtagctgaggaatttgttgaggataaggatgtttgcaaaaattatgaagaggcaaaagatctcttcttgggcaagtttttgtatgagtattcaagaaggcaccctgattactctgtcgttctgctgctgagacttggcaaggcctatgaagccaccctgaaaaagtgctgtgccactgatgaccctcacgcatgctatgccaaagtgcttgatgagtttcagcctcttgtggatgaacccaagaatttagtgaaacaaaactgtgaactctatgagcagcttggtgactacaacttccaaaatgcgctcctagttcgttataccaagaaagtacctcaagtgtcaactccaactctcgtggaaatatcaagaagcctaggaaaagtgggcagcaagtgctgtaagcatcctgaagcagaaagactgccttgtgttgaagattatctgtccgtggtcctgaacaggttgtgcgtgttgcatgagaagacaccagtgagtgagaaagtcaccaaatgctgctcagagtcattggtcgacagacgaccatgctttagcgccctgggccccgatgaaacatacgtccccaaagaatttaatgctgaaacattcaccttccatgcggacatatgcactcttccagaaacggagaggaaaatcaagaaacaaacggcacttgttgagttggtgaaacacaagccccacgcaacaaatgatcaactgaaaactgttgttggagagttcacagctttgttagacaagtgctgcagtgctgaagacaaggaggcctgctttgctgtggagggtccaaaacttgttgaatcaagtaaagctaccttaggctaaaaaatcacagccacaataatctcagcctaccctgagaataagagaagagaaatgaagacccagagcctattcatctgtttttcttttctgttgatataaaaccaacag

SEQ ID NO: 25 - protein, 608 aa, Oryctolagus cuniculus
MKWVTFISLLFLFSSAYSRGVFRREAHKSEIAHRFNDVGEEHFIGLVLITFSQYLQKCPYEEHAKLVKEVTDLAKACVADESAANCDKSLHDIFGDKICALPSLRDTYGDVADCCEKKEPERNECFLHHKDDKPDLPPFARPEADVLCKAFHDDEKAFFGHYLYEVARRHPYFYAPELLYYAQKYKAILTECCEAADKGACLTPKLDALEGKSLISAAQERLRCASIQKFGDRAYKAWALVRLSQRFPKADFTDISKIVTDLTKVHKECCHGDLLECADDRADLAKYMCEHQETISSHLKECCDKPILEKAHCIYGLHNDETPAGLPAVAEEFVEDKDVCKNYEEAKDLFLGKFLYEYSRRHPDYSVVLLLRLGKAYEATLKKCCATDDPHACYAKVLDEFQPLVDEPKNLVKQNCELYEQLGDYNFQNALLVRYTKKVPQVSTPTLVEISRSLGKVGSKCCKHPEAERLPCVEDYLSVVLNRLCVLHEKTPVSEKVTKCCSESLVDRRPCFSALGPDETYVPKEFNAETFTFHADICTLPETERKIKKQTALVELVKHKPHATNDQLKTVVGEFTALLDKCCSAEDKEACFAVEGPKLVESSKATLG

SEQ ID NO: 26 - DNA, 1920 bp, Mus musculus
atgaagtgggtaacctttctcctcctcctcttcgtctccggctctgctttttccaggggtgtgtttcgccgagaagcacacaagagtgagatcgcccatcggtataatgatttgggagaacaacatttcaaaggcctagtcctgattgccttttcccagtatctccagaaatgctcatacgatgagcatgccaaattagtgcaggaagtaacagactttgcaaagacgtgtgttgccgatgagtctgccgccaactgtgacaaatcccttcacactctttttggagataagttgtgtgccattccaaacctccgtgaaaactatggtgaactggctgactgctgtacaaaacaagagcccgaaagaaacgaatgtttcctgcaacacaaagatgacaaccccagcctgccaccatttgaaaggccagaggctgaggccatgtgcacctcctttaaggaaaacccaaccacctttatgggacactatttgcatgaagttgccagaagacatccttatttctatgccccagaacttctttactatgctgagcagtacaatgagattctgacccagtgttgtgcagaggctgacaaggaaagctgcctgaccccgaagcttgatggtgtgaaggagaaagcattggtctcatctgtccgtcagagaatgaagtgctccagtatgcagaagtttggagagagagcttttaaagcatgggcagtagctcgtctgagccagacattccccaatgctgactttgcagaaatcaccaaattggcaacagacctgaccaaagtcaacaaggagtgctgccatggtgacctgctggaatgcgcagatgacagggcggaacttgccaagtacatgtgtgaaaaccaggcgactatctccagcaaactgcagacttgctgcgataaaccactgttgaagaaagcccactgtcttagtgaggtggagcatgacaccatgcctgctgatctgcctgccattgctgctgattttgttgaggaccaggaagtgtgcaagaactatgctgaggccaaggatgtcttcctgggcacgttcttgtatgaatattcaagaagacaccctgattactctgtatccctgttgctgagacttgctaagaaatatgaagccactctggaaaagtgctgcgctgaagccaatcctcccgcatgctacggcacagtgcttgctgaatttcagcctcttgtagaagagcctaagaacttggtcaaaaccaactgtgatctttacgagaagcttggagaatatggattccaaaatgccattctagttcgctacacccagaaagcacctcaggtgtcaaccccaactctcgtggaggctgcaagaaacctaggaagagtgggcaccaagtgttgtacacttcctgaagatcagagactgccttgtgtggaggactatctgtctgcaatcctgaaccgtgtgtgtctgctgcatgagaagaccccagtgagtgagcatgttaccaagtgctgtagtggatccctggtggaaaggcggccatgcttctctgctctgacagttgatgaaacatatgtccccaaagagtttaaagctgagaccttcaccttccactctgatatctgcacacttccagagaaggagaagcagattaagaaacaaacggctcttgctgagctggtgaagcacaagcccaaggctacagcggagcaactgaagactgtcatggatgactttgcacagttcctggatacatgttgcaaggctgctgacaaggacacctgcttctcgactgagggtccaaaccttgtcactagatgcaaagacgccttagcctaaacacaccacaaccacaaccttctcaggctaccctgacacatgaaagggcgaattccagcacactggcggccgttactagtggatccgagctcg

SEQ ID NO: 27 - protein, 607 aa, Mus musculus
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDEYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 28 - DNA, 1956 bp, Rattus norvegicus
atgaagtgggtaacctttctcctcctcctcttcatctccggttctgccttttctaggggtgtgtttcgccgagaagcacacaagagtgagatcgcccatcggtttaaggacttaggagaacagcatttcaaaggcctagtcctgattgccttttcccagtatctccagaaatgcccatatgaagagcatatcaaattggtgcaggaagtaacagactttgcaaaaacatgtgtcgctgatgagaatgccgaaaactgtgacaagtccattcacactctcttcggagacaagttatgcgccattccaaagcttcgtgacaactacggtgaactggctgactgctgtgcaaaacaagagcccgaaagaaacgagtgtttcctgcagcacaaggatgacaaccccaacctgccacccttccagaggccggaggctgaggccatgtgcacctccttccaggagaaccctaccagctttctgggacactatttgcatgaagttgccaggagacatccttatttctatgccccagaactcctttactatgctgagaaatacaatgaggttctgacccagtgctgcacagagtctgacaaagcagcctgcctgacaccgaagcttgatgccgtgaaagagaaagcactggtcgcagctgtccgtcagaggatgaagtgctccagtatgcagagatttggagagagagccttcaaagcctgggcagtagctcgtatgagccagagattccccaatgctgagttcgcagaaatcaccaaattggcaacagacgttaccaaaatcaacaaggagtgctgtcacggcgacctgttggaatgcgcggatgacagggcagaacttgccaagtacatgtgtgagaaccaggccactatctccagcaaactgcaggcttgctgtgataagccagtgctgcagaaatcccagtgtctcgctgagacagaacatgacaacattcctgccgatctgccctcaatagctgctgactttgttgaggataaggaagtgtgtaagaactatgctgaggccaaggatgtcttcctgggcacgtttttgtatgaatattcaagaaggcaccccgattactccgtgtccctgctgctgagacttgctaagaaatatgaagccacactggagaagtgctgtgctgaaggcgatcctcctgcctgctacggcacagtgcttgcagaatttcagcctcttgtagaagaacctaagaacttggtcaaaactaactgtgagctttacgagaagcttggagagtatggattccaaaacgccgttctggttcgatacacccagaaagcacctcaggtgtcgaccccaactctcgtggaggcagcaagaaacctgggaagagtgggcaccaagtgttgtacccttcctgaagctcagagactgccctgtgtggaagactatctgtctgccatcctgaaccgtctgtgtgtgctgcatgagaagaccccagtgagcgagaaggtcaccaagtgctgtagtgggtccttggtggaaagacggccatgtttctctgctctgacagttgacgagacatatgtccccaaagagtttaaagctgagaccttcaccttccactctgatatctgcacactcccagacaaggagaagcagataaagaagcaaacggctctcgctgagctggtgaaacacaagcccaaggccacagaagatcagctgaagacggtgatgggtgacttcgcacaattcgtggacaagtgttgcaaggctgccgacaaggataactgcttcgccactgaggggccaaaccttgttgctagaagcaaagaagccttagcctaaacacatcacaaccatctcaggctaccctgagaaaaaaagacatgaagactcaggactcatctcttctgttggtgtaaaaccaacaccctaaggaacacaaatttctttgaacatttgacttcttttctc

SEQ ID NO: 29 - protein, 608 aa, Rattus norvegicus
MKWVTFLLLLFISGSAFSRGVFRREAHKSEIAHRFKDLGEQHFKGLVLIAFSQYLQKCPYEEHIKLVQEVTDFAKTCVADENAENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESDKAACLTPKLDAVKEKALVAAVRQRMKCSSMQRFGERAFKAWAVARMSQRFPNAEFAEITKLATDVTKINKECCHGDLLECADDRAELAKYMCENQATISSKLQACCDKPVLQKSQCLAETEHDNIPADLPSIAADFVEDKEVCKNYAEAKDVFLGTFLYEYSRRHPDYSVSLLLRLAKKYEATLEKCCAEGDPPACYGTVLAEFQPLVEEPKNLVKTNCELYEKLGEYGFQNAVLVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAADKDNCFATEGPNLVARSKEALA

SEQ ID NO: 30 - DNA, 1624 bp, Bos taurus
gcgtgtccaggttctgccacgtgccacctgccgaccctgaagaagatggcctacccgtggaccttcaccttcctctgtggtttgctggcagccaacctggtaggagccaccttaagccctcctgtggttctcagtctcagcacagaagtcatcaagcaaatgctggctcagaaactgaagaatcacgatgttaccaacaccctgcagcagctgccactgctcactgccatggaggaggagtcgtccaggggcattttcggcaacctggtgaaatccatcctgaagcatattctctggatgaaagtcacctcagccagcatcggtcagctgcaggtgcagcccctggccaacggccggcagctgatggtcaaggcccccctggacgtggtggctggattcaacgtgccccttttcaagaccgttgtggagctgcatgtggaggtggaggcccaagccatcatccacgtggagactagggagaaggaccacgcccgcctggtcctcagcgagtgctccaacaccggcgggagcctgcgcgtcagcctgctgcacaagctctccttcctgctcaaatgcttagccgacaaggtcataagccttctgacgccagcgccccctaaactggtgaaaagcgagctgtgtcctgtgctcaaggcgggctttgaggacatgcgtggggaactcttgaatctgacgaaggtgcccatgtctctcaactctgagcacctgaagcttgattttatttctcctgtcatcgaccacagtgttgtccatctcatcctgggggccaggttgttcaactcagaaggaaaggtgactaagctgttcaatgttgctggggattccctgaatctgcccaccctgaaccagacccctttcaggctcaccgtgaggaaggatgtggtggtcgctatcatagctgccttgatccattcgggaaaactcacagtcctgttggactatgtgcttcctgaggtagcccgccagctgaggtcaagcatcaaggtgatcgacgaaacggcagcagcgcagctggggcccacacagatcgtgaagatcatgagtcagacgaccccaatgctcattctggaccagggcaatgccaaggtggcccaactgatcgtgctggaaatattcgccaccgataaagacagccgccccctcttcaccctgggcatcgaagcctcctcggacattcagttttacgtcgaagatggcctacttgtgttcagctttaacgaaatcagagctgatcggatccatctgatgaactcagacatcggtgtgttcaaccctaagcttctgaacaacatcaccaccaagatcctcacctccatcctgctgccaaacgagaatggcaaattaagatctgggatcccagtgtcaatggtgaaaaacttgggatttaagtcgatttcattgtctctgaccaaggaagcccttgtggtcacccaagcctcctcttagaacctcagcccactttcctctctcccagtgaagacttgcactgtggtcctccagggaaggctgtgtctcaatgagagtgtgggagccagcgctgtaatctgtccctccctacaatgaataaactttgtgaatcttgcagtccaaaaaaaaaaaaaaa

SEQ ID NO: 31 - protein, 473 aa, Bos taurus
MAYPWTFTFLCGLLAANLVGATLSPPVVLSLSTEVIKQMLAQKLKNHDVTNTLQQLPLLTAMEEESSRGIFGNLVKSILKHILWMKVTSASIGQLQVQPLANGRQLMVKAPLDVVAGFNVPLFKTVVELHVEVEAQAIIHVETREKDHARLVLSECSNTGGSLRVSLLHKLSFLLKCLADKVISLLTPAPPKLVKSELCPVLKAGFEDMRGELLNLTKVPMSLNSEHLKLDFISPVIDHSVVHLILGARLFNSEGKVTKLFNVAGDSLNLPTLNQTPFRLTVRKDVVVAIIAALIHSGKLTVLLDYVLPEVARQLRSSIKVIDETAAAQLGPTQIVKIMSQTTPMLILDQGNAKVAQLIVLEIFATDKDSRPLFTLGIEASSDIQFYVEDGLLVFSFNEIRADRIHLMNSDIGVFNPKLLNNITTKILTSILLPNENGKLRSGIPVSMVKNLGFKSISLSLTKEALVVTQASS

SEQ ID NO: 32 - DNA, 531 bp, Sus scrofa
atgatgagggctctgctcctggccattggcctcggcctcgttgctgccctgcaggcccaggagttcccggccgtggggcagccgctgcaggatctgctggggagatggtatctgaaggccatgacctcggacccggagattcccgggaagaagcccgagtcggtgacccccctgattctcaaggccctggaggggggcgacctggaagcccagataacctttctgattgacggtcagtgccaggacgtgacactggtcctaaagaaaaccaaccagcccttcacgttcacggcctatgacggcaagcgcgtggtgtacatcttaccgtccaaggtgaaggaccactacattctctactgcgagggtgagctggacgggcaggaggtccgcatggcgaagctcgtgggaagagacccagagaacaacccagaggctttggaggagttcaaggaggtggcaagagccaaagggctaaacctggacatcgtcaggccccagcaaagcgaaacctgctctccaggagggaactag

SEQ ID NO: 33 - protein, 176 aa, Sus scrofa
MMRALLLAIGLGLVAALQAQEFPAVGQPLQDLLGRWYLKAMTSDPEIPGKKPESVTPLILKALEGGDLEAQITFLIDGQCQDVTLVLKKTNQPFTFTAYDGKRVVYILPSKVKDHYILYCEGELDGQEVRMAKLVGRDPENNPEALEEFKEVARAKGLNLDIVRPQQSETCSPGGN

SEQ ID NO: 34 - DNA, 1611 bp, Homo sapiens
gcccgggagaggagaggagcgggccgaggactccagcgtgcccagatggccggcccgtggaccttcacccttctctgtggtttgctggcagccaccttgatccaagccaccctcagtcccactgcagttctcatcctcggcccaaaagtcatcaaagaaaagctgacacaggagctgaaggaccacaacgccaccagcatcctgcagcagctgccgctgctcagtgccatgcgggaaaagccagccggaggcatccctgtgctgggcagcctggtgaacaccgtcctgaagcacatcatctggctgaaggtcatcacagctaacatcctccagctgcaggtgaagccctcggccaatgaccaggagctgctagtcaagatccccctggacatggtggctggattcaacacgcccctggtcaagaccatcgtggagttccacatgacgactgaggcccaagccaccatccgcatggacaccagtgcaagtggccccacccgcctggtcctcagtgactgtgccaccagccatgggagcctgcgcatccaactgctgcataagctctccttcctggtgaacgccttagctaagcaggtcatgaacctcctagtgccatccctgcccaatctagtgaaaaaccagctgtgtcccgtgatcgaggcttccttcaatggcatgtatgcagacctcctgcagctggtgaaggtgcccatttccctcagcattgaccgtctggagtttgaccttctgtatcctgccatcaagggtgacaccattcagctctacctgggggccaagttgttggactcacagggaaaggtgaccaagtggttcaataactctgcagcttccctgacaatgcccaccctggacaacatcccgttcagcctcatcgtgagtcaggacgtggtgaaagctgcagtggctgctgtgctctctccagaagaattcatggtcctgttggactctgtgcttcctgagagtgcccatcggctgaagtcaagcatcgggctgatcaatgaaaaggctgcagataagctgggatctacccagatcgtgaagatcctaactcaggacactcccgagttttttatagaccaaggccatgccaaggtggcccaactgatcgtgctggaagtgtttccctccagtgaagccctccgccctttgttcaccctgggcatcgaagccagctcggaagctcagttttacaccaaaggtgaccaacttatactcaacttgaataacatcagctctgatcggatccagctgatgaactctgggattggctggttccaacctgatgttctgaaaaacatcatcactgagatcatccactccatcctgctgccgaaccagaatggcaaattaagatctggggtcccagtgtcattggtgaaggccttgggattcgaggcagctgagtcctcactgaccaaggatgcccttgtgcttactccagcctccttgtggaaacccagctctcctgtctcccagtgaagacttggatggcagccatcagggaaggctgggtcccagctgggagtatgggtgtgagctctatagaccatccctctctgcaatcaataaacacttgcctgtgatgcctgc

SEQ ID NO: 35 - protein, 484 aa, Homo sapiens
MAGPWTFTLLCGLLAATLIQATLSPTAVLILGPKVIKEKLTQELKDHNATSILQQLPLLSAMREKPAGGIPVLGSLVNTVLKHIIWLKVITANILQLQVKPSANDQELLVKIPLDMVAGFNTPLVKTIVEFHMTTEAQATIRMDTSASGPTRLVLSDCATSHGSLRIQLLHKLSFLVNALAKQVMNLLVPSLPNLVKNQLCPVIEASFNGMYADLLQLVKVPISLSIDRLEFDLLYPAIKGDTIQLYLGAKLLDSQGKVTKWFNNSAASLTMPTLDNIPFSLIVSQDVVKAAVAAVLSPEEFMVLLDSVLPESAHRLKSSIGLINEKAADKLGSTQIVKILTQDTPEFFIDQGHAKVAQLIVLEVFPSSEALRPLFTLGIEASSEAQFYTKGDQLILNLNNISSDRIQLMNSGIGWFQPDVLKNIITEIIHSILLPNQNGKLRSGVPVSLVKALGFEAAESSLTKDALVLTPASLWKPSSPVSQ

SEQ ID NO: 36 - DNA, 925 bp, Mus musculus
ctgaacccagagagtatataagaacaagcaaaggggctggggagtggagtgtagccacgatcacaagaaagacgtggtcctgacagacagacaatcctattccctaccaaaatgaagatgctgctgctgctgtgtttgggactgaccctagtctgtgtccatgcagaagaagctagttctacgggaaggaactttaatgtagaaaagattaatggggaatggcatactattatcctggcctctgacaaaagagaaaagatagaagataatggcaactttagactttttctggagcaaatccatgtcttggagaattccttagttcttaaattccatactgtaagagatgaagagtgctcggaattatctatggttgctgacaaaacagaaaaggctggtgaatattctgtgacgtatgatggattcaatacatttactatacctaagacagactatgataactttcttatggctcatctcattaacgaaaaggatggggaaaccttccagctgatggggctctatggccgagaaccagatttgagttcagacatcaaggaaaggtttgcacaactatgtgagaagcatggaatccttagagaaaatatcattgacctatccaatgccaatcgctgcctccaggcccgagaatgaagaatggcctgagcctccagtgttgagtggagacttctcaccaggactccaccatcatcccttcctatccatacagcatccccagtataaattctgtgatctgcattccatcctgtctcactgagaagtccaattccagtctatccacatgttacctaggatacctcatcaagaatcaaagacttctttaaatttttctttgatatacccatgacaatttttcatgaatttcttcctcttcctgttcaataaatgattacccttgcactta

SEQ ID NO: 37 - protein, 180 aa, Mus musculus
MKMLLLLCLGLTLVCVHAEEASSTGRNFNVEKINGEWHTIILASDKREKIEDNGNFRLFLEQIHVLENSLVLKFHTVRDEECSELSMVADKTEKAGEYSVTYDGFNTFTIPKTDYDNFLMAHLINEKDGETFQLMGLYGREPDLSSDIKERFAQLCEKHGILRENIIDLSNANRCLQARE

SEQ ID NO: 38 - DNA, 405 bp, Helicoverpa assulta
atgaattttgctaagcccttagaagactgtaagaaagagatggatctcccagactcggtgacgacagacttctacaacttctggaaggaaggctacgagttcacgaacagacagacgggctgcgccatcctctgcctctcctccaagctagagctgctggaccaggagatgaagctgcaccacggcaaggcgcaggagttcgccaagaaacatggcgctgacgatgctatggctaagcagctggtagacctgatccacggctgctcgcggtctactcctgacgtgacagacgatccctgtatgaaggccctcaacgtggccaagtgcttcaaggccaagatacacgagctcaactgggcgcccagcatggacctcgtcgtcggagaagtcttggccgaagtttag

SEQ ID NO: 39 - protein, 134 aa, Helicoverpa assulta
MNFAKPLEDCKKEMDLPDSVTTDFYNFWKEGYEFTNRQTGCAILCLSSKLELLDQEMKLHHGKAQEFAKKHGADDAMAKQLVDLIHGCSRSTPDVTDDPCMKALNVAKCFKAKIHELNWAPSMDLVVGEVLAEV

SEQ ID NO: 40 - DNA, 760 bp, Sesamia nonagrioides
attattcaaaatggctgattcaagatggtggttcgcgagtttcatctgcgtcattattatgacaagttcggtgatgtcttccaaggagttggtctccaaaatgagttccgggttctcgaaggttttggatcagtgtaaagctgagctgaacgtgggcgaacacataatgcaagacatgtacaacttctggcgcgaggagtacgagctggtgaaccgcgacctgggatgcatggtgatgtgcatggcctccaagttggacctggtaggagacgaccagaagatgcaccatggaaaggccgaggagtttgccaagagtcatggagctgatgacgagctggctaagcagctggtgggcatcatccatgcctgcgagacgcagcaccaagccatcgaggatccctgcagccgcacgctggaggtggccaagtgcttccgctcgaagatgcacgagctgaagtgggccccgcccatggaggtcgccatagaagagattatgacagctgtttaggtggaatatgggatagaaaggggaggaaggagtgaaatagggccttttcaattcttatttaaaaaatgtaataataatactaaaggtgccggtggtttattagtttcttattgattataacttattattactaacatctctcgcaactcgtcagtttcttattattatttaataataaccggtgttagaattatttttattaaaataaagtatattattttagtccaaaaaaaaaaaaaaaaaaaaaaaaaa

SEQ ID NO: 41 - protein, 165 aa, Sesamia nonagrioides
MADSRWWFASFICVIIMTSSVMSSKELVSKMSSGFSKVLDQCKAELNVGEHIMQDMYNFWREEYELVNRDLGCMVMCMASKLDLVGDDQKMHHGKAEEFAKSHGADDELAKQLVGIIHACETQHQAIEDPCSRTLEVAKCFRSKMHELKWAPPMEVAIEEIMTAV

SEQ ID NO: 42 - DNA, 1078 bp, Sesamia nonagrioides
  misc_feature (462)..(462): n is a, c, g, or t
aatggcgctgcatcgatcgcccatcatgtcggcacgcttggcgctggtactgatcgccagtctgttcatcgtcgtgaaatgttctcaagaagtcatgaagaatctgacccatcatttctctaagcctttggaagactgtaagaaggagatggacctcccggactcagtgatcacagatttctacaatttctggaaagaaggctacgagttcacgagcagacatacaggctgtgccatactctgcctctcatctaagctggaactgctcgatccagaccttaagttgcatcatggaaaggcgcaggagttcgcgcagaaacatggcgctgacgaggccatggcgaagcagctggtaggcctgatccacggctgtatggagacaatccgcgaaccggccgacgacccctgcgtgagggctcagaacgtagtcatgtgcttcaaggccaagatacatgagctganctgggcgcctagcttggacctcatcgtgggagaagtcttggctgaagtctagcatgatgcccttggttccgtgatatacctttatcttctcttcgtcatagaaggccatcattgcatgtgatagtgatgttgttgttttgaatgcaaaacatagtttcatctttttcatttgttttgcttgagtgttttcagctagagacattacgtaaatcaaagtcttttttatcaaatatcattctctgttaagaaaccaataaccagtgctcagacaacattaatgttatgtgcggttgtaatgtaatgcaatgcttatgacctgcaggaataaatgcaaataagtttatatctacattacattatgtttatacattacagtacattgtgttatacaagtctgatttgtttctatctctactttaacgacaaggcttgttcaatggactacagatatttctacagttagttatttgattaatatttaataattcttgtgaagagtctcctgtctcgccagttctcatcaaagggagtgggtacattgtaagttgcaagttctggatgtcatattaataaagaatacatctttacaaaaaaaaaaaaaaaaaaaaaaaaaaaa

SEQ ID NO: 43 - protein, 170 aa, Sesamia nonagrioides
  misc_feature (154)..(154): Xaa can be any naturally occurring amino acid
MALHRSPIMSARLALVLIASLFIVVKCSQEVMKNLTHHFSKPLEDCKKEMDLPDSVITDFYNFWKEGYEFTSRHTGCAILCLSSKLELLDPDLKLHHGKAQEFAQKHGADEAMAKQLVGLIHGCMETIREPADDPCVRAQNVVMCFKAKIHELXWAPSLDLIVGEVLAEV

SEQ ID NO: 44 - DNA, 1077 bp, Spodoptera exigua
acgcgggggcagataacaagatggcgggcgcaaaatggcggtttgtctgtgttgtgttcgcgctgtacctgaccagcgccgcgctgggctcgcaggagctcatgatgaagatgactaagggattcacgaaagtcgtcgatgagtgcaaagctgagcttaacgcgggggagcacatcatgcaggacatgtacaactactggcgcgaagactaccagctcattaaccgggacttgggctgcatgatcctgtgcatggcaaagaagttggacctcatggaagaccagaagatgcaccacgggaagacagaagaattcgccaagagtcatggcgctgatgacgaggttgccaagaagctggtgagcataatccacgaatgcgagcagcagcacgccggcatagcggacgattgcatgagggtgttggagatatccaaatgcttccgcaccaagattcacgagctcaaatgggcacccaacatggaggtcattatggaagaggtgatgaccgccgtgtagacacgagggaaccaggaaacaatgtcattttagggaaaactgctgcagttgttggagtgtcacgcgggataatgatctgcagcgttagcaaaactgatgtacatacttgtaatcgagaatgctatggcaaacgaaacaaatgtatttggagatttatcagtttgaatacgttgtgtggcgcgtgggcaatagtgaatctatacgtatgaacaacatttgtttccttttatttagcgttaacgatcacaagttgtactgaacgataactaaagctcataatggttctaagattatctctagattgcagggctataactggaaagggtttcgtgtcatttcgtttcatgtcgctgattgatcaacactttctaacacctttacacaattctcttcaatcgtcggagttattctacttcacccagaagtgaaattgttgtatcattatcctggctctttattcagtgaaactatgtagctgtataagtattatttatttcctctttaggttcttggttaattaaagtgtttcaattcatgaaaaaaaaaaaaaaaaaaaaaaaaa

SEQ ID NO: 45 - protein, 164 aa, Spodoptera exigua
MAGAKWRFVCVVFALYLTSAALGSQELMMKMTKGFTKVVDECKAELNAGEHIMQDMYNYWREDYQLINRDLGCMILCMAKKLDLMEDQKMHHGKTEEFAKSHGADDEVAKKLVSIIHECEQQHAGIADDCMRVLEISKCFRTKIHELKWAPNMEVIMEEVMTAV

SEQ ID NO: 46 - DNA, 737 bp, Spodoptera exigua
acgcgggggaccatgtcggtgagggtggcgctggtggtggccgccagtatgctggtagtggtacaggcgtcgcaagatgtcatgaagaacttggccatcaatttcgcgaaacctttggatgactgtaagaaggagatggacctgccagactcggtgacgaccgacttctacaacttctggaaggaaggatacgagctgacgaacagacagaccggctgtgctatcctgtgtctctcttcgaagttggagattcttgaccaagaactgaacctgcatcacggcagggcgcaggagtttgctatgaaacacggcgctgacgagaccatggcgaagcagatagtggacatgatccacacttgtgcgcagtctactcccgacgtagcggcggacccttgcatgaagaccctgaatgtagccaagtgcttcaagttgaagatacacgagctcaactgggcgcccagcatggagctcatcgtgggagaagtgctggctgaagtgtaacttgaatcactcaagacctttaagctggccttcattatgtgaggtcttcataaacatatctttgacgtctcggctcgttgaacggaccccagattaggttaggttggtcgtgaagcatctcctggctcgccagttcgcctcagagggtgtgggtagtagtaggctgcagcaagatgtcaaatattgttcaatatactgtacatcattaaaaaaaaaaaaaaaaaaaaaaaaaaaaa

SEQ ID NO: 47 - protein, 162 aa, Spodoptera exigua
MSVRVALVVAASMLVVVQASQDVMKNLAINFAKPLDDCKKEMDLPDSVTTDFYNFWKEGYELTNRQTGCAILCLSSKLEILDQELNLHHGRAQEFAMKHGADETMAKQIVDMIHTCAQSTPDVAADPCMKTLNVAKCFKLKIHELNWAPSMELIVGEVLAEV

SEQ ID NO: 48 - DNA, 546 bp, Drosophila melanogaster
agttcaactttagcaatttttggggagaagcaaaaatggttgcaaggcattttagtttttttttagcactactcatactatatgatttaattcctagtaatcaaggagtggaaattaatcctacgatcataaagcaggtgagaaagctgcgaatgcgatgcttaaatcagacaggagcttctgtagatgtgattgacaagtcggtgaaaaatagaatactacctacagatcccgagatcaagtgttttctctactgcatgtttgatatgttcggattgattgattcacaaaacataatgcacttggaagcactgttggaggttttacccgaggaaatacacaaaacgattaacggattagtcagttcatgtggaactcagaagggaaaagatggctgtgataccgcttatgaaaccgtcaagtgctacattgctgtaaacggaaaattcatatgggaagagataatagtgctacttgggtagcgctaaccaacctaaatatatcccgatccacgattcccaagagcagcaaacagcgcaggatgcg

SEQ ID NO: 49 - protein, 148 aa, Drosophila melanogaster
MVARHFSFFLALLILYDLIPSNQGVEINPTIIKQVRKLRMRCLNQTGASVDVIDKSVKNRILPTDPEIKCFLYCMFDMFGLIDSQNIMHLEALLEVLPEEIHKTINGLVSSCGTQKGKDGCDTAYETVKCYIAVNGKFIWEEIIVLLG

SEQ ID NO: 50 - DNA, 846 bp, Drosophila melanogaster
aaagcaaattcaattgtgactgcggttgtcaaacaattcttgcgtgtcgggtgtgtgcagtatcgagttctggccataactacttctgctaaaagcgaacgagcttgtttttgttttattcagagctcgcaaataaggccgagccagggcacaatttttgctgtttcacggatggaccaggaaggaccacgcagcagcggaaaggagcgaaacggaaagagccacattaaaatggctttgaatggctttggtcggcgtgtcagtgcgtctgtccttttaatcgccttgtcgctgctcagcggagcgctgatcctgccgccggctgcggcgcagcgtgacgagaactatccaccgccgggcatcctgaaaatggccaagcccttccacgacgcgtgtgtggagaagacgggcgtaaccgaggctgccatcaaggagttcagcgatggggagattcacgaggacgagaagctcaaatgctacatgaactgcttcttccacgagatcgaagtggtggacgacaatggggacgtgcatctggagaagctcttcgccacggtaccgctctccatgcgcgacaagctgatggagatgtccaagggctgcgtccatccggagggcgatacgctgtgccacaaggcctggtggttccaccagtgctggaaaaaggccgatcccaagcactacttcttgccgtgaacacctgggccacctttcagcccagttccagttccatggtccgtggaccacccgttgccgaccccgctctatttatgtggtagtttagtttctgctagttttcaatagctgtcgagtaataaacgtaggcgagttgtgcatgcaagctaa

SEQ ID NO: 51 - protein, 154 aa, Drosophila melanogaster
MALNGFGRRVSASVLLIALSLLSGALILPPAAAQRDENYPPPGILKMAKPFHDACVEKTGVTEAAIKEFSDGEIHEDEKLKCYMNCFFHEIEVVDDNGDVHLEKLFATVPLSMRDKLMEMSKGCVHPEGDTLCHKAWWFHQCWKKADPKHYFLP

SEQ ID NO: 52 - DNA, 1104 bp, Drosophila melanogaster
tcacaatcactcatctcacccagagctgttgatcgatttaattacaagcgggatttctcatctctcattttgcatttagcattttgcattttcatttccatttccactagccatagccattcccaattctatatccccggcatttgcagcgatttcatgccagtcaccaattaagcaggtaagtggagatcggtgggccatctcatctggcagcggcagttccagcggggtgtcactcgttcacacgatgcccagtcgagggcatctccgccggattccgtcccatcccgtccagagcggcggagtgaagtggagtgccatgtgccatgtgctgcccatgtagttcataattgcgcgtaattgccggagctgcttgagacgcagctggagatcggcgatggatccgatctgccaaatcaatcacgggactcggcttaggcaatagctcctataaaacgccgacgttgccggcgattcgcatccaagtcagagttcgcacgtcgcgcagttcaatcgcaaatcgaaatgtcgcatctggttcacctgaccgtcctgctcctagtgggcatcctctgcctgggcgccaccagcgccaagccgcacgaggagatcaacagggaccacgccgccgagctggccaacgagtgcaaggctgagacgggagccaccgatgaggatgtggagcagctgatgagccacgacctgcccgagagacacgaggccaagtgcctgcgcgcctgcgtgatgaaaaagctgcagataatggatgaatccggtaagctgaacaaggaacacgccatcgagttggtcaaggtcatgagcaagcacgatgcagagaaggaagacgctcccgccgaggtggtggccaagtgcgaggccatcgagacacccgaggatcattgcgacgctgccttcgcctacgaggaatgcatttacgagcaaatgaaggagcatggactcgagctggaggagcactgagaacagatttgagacccatgacgaccccccgttactgtatcacaagcgcccttctggaatataaccatcttttttttttatgtgtatactatgaattaagtacttgataaactgagaaactgcagg

SEQ ID NO: 53 - protein, 150 aa, Drosophila melanogaster
MSHLVHLTVLLLVGILCLGATSAKPHEEINRDHAAELANECKAETGATDEDVEQLMSHDLPERHEAKCLRACVMKKLQIMDESGKLNKEHAIELVKVMSKHDAEKEDAPAEVVAKCEAIETPEDHCDAAFAYEECIYEQMKEHGLELEEH

SEQ ID NO: 54 - DNA, 579 bp, Drosophila melanogaster
agcactttgtttgttcaagatgtattccgcgttagttagagcttgtgctgtcattgcttttctgatcttgagcccgaattgtgccagggctctacaggatcacgccaaggataatggtgatattttcatcataaactatgatagtttcgatggcgatgtggatgacatatccaccaccacttcagctcctagagaggctgactacgtagattttgacgaggttaatcgtaactgcaatgctagtttcataacgtcgatgaccaatgtcttgcagtttaataacactggggatttgccagatgacaaggataaggtaaccagcatgtgctattttcactgctttttcgaaaagtccggtttgatgacggactataagttaaatacggatctggtgcgcaaatatgtttggccagccactggcgattccgttgaggcctgcgaagctgaaggcaaggacgagacgaatgcttgcatgcggggctatgccatcgtcaagtgcgtgtttactagagccctcacggatgctagaaacaaacccactgtatgaataacatcaaaggtcacatctcggacttatca

SEQ ID NO: 55 - protein, 175 aa, Drosophila melanogaster
MYSALVRACAVIAFLILSPNCARALQDHAKDNGDIFIINYDSFDGDVDDISTTTSAPREADYVDFDEVNRNCNASFITSMTNVLQFNNTGDLPDDKDKVTSMCYFHCFFEKSGLMTDYKLNTDLVRKYVWPATGDSVEACEAEGKDETNACMRGYAIVKCVFTRALTDARNKPTV

SEQ ID NO: 56 - DNA, 494 bp, Drosophila melanogaster
aagttccgttcagacacaccgacctagcatcatgcagtctactccaatcattctggtggcaatcgtccttctcggcgccgcactggtgcgagcctttgacgagaaggaggccctggccaagctgatggagtcagccgagagctgcatgccggaagtgggggccaccgatgccgatctgcaggaaatggtcaagaagcagccagccagcacatatgccggcaagtgcctgcgcgcctgcgtgatgaagaacatcggaattctggacgccaacggaaaactggacacggaggcaggtcacgagaaggccaagcagtacacgggcaacgatccggccaagctaaagattgccctggagatcggcgacacctgtgccgccatcactgtgccggatgatcactgcgaggccgccgaagcctatggcacttgcttcaggggcgaggccaagaaacatggactcttgtaatcattgatgcagcgctacccacctggacacg

SEQ ID NO: 57 - protein, 143 aa, Drosophila melanogaster
MQSTPIILVAIVLLGAALVRAFDEKEALAKLMESAESCMPEVGATDADLQEMVKKQPASTYAGKCLRACVMKNIGILDANGKLDTEAGHEKAKQYTGNDPAKLKIALEIGDTCAAITVPDDHCEAAEAYGTCFRGEAKKHGLL

(19) United States
(12) Patent Application Publication
(10) Pub. No.: US 2009/0136968 A1
(43) Pub. Date: May 28, 2009

(54) Novel in vitro methods for studying receptors recognizing volatile compounds

(75) Inventor: Frederic Sallman, Brussels (BE)

Correspondence Address: EDWARDS ANGELL PALMER & DODGE LLP, P.O. BOX 55874, BOSTON, MA, 02205, US

(73) Assignee: Chemcom S. A., Brussel (BE)

(21) Appl. No.: 12/152,267

(22) Filed: May 12, 2008

Related U.S. Application Data
(63) Continuation of application No. 11/893,459, filed on Aug. 16, 2007, now abandoned.
Application No. 11/893,459 is a continuation of application No. PCT/US2006/001330, filed on Feb. 14, 2006.

(30) Foreign Application Priority Data
Feb. 17, 2005 (EP) 05447035.6

(51) Int. Cl. G01N 33/566

(52) U.S. Cl. 435/7.2

(57) ABSTRACT

The present invention in particular relates to in vitro methods to identify and/or confirm the binding and/or function of a volatile compound onto a membrane-integrated receptor using volatile-compound-Binding Protein (BP) or compositions thereof. Additionally, the present invention relates to kits comprising receptor proteins recognizing volatile compounds or a candidate receptor for said compound; and; BP, a complex or composition thereof. Said kits may be used to identify and/or confirm the binding and/or function of volatile compounds onto a membrane-integrated receptor.

This application is a Continuation of Ser. No. 11/893,459, Filed on Aug. 16, 2007, which is a Continuation of PCT/US2006/001330, filed on Feb. 14, 2006, which claims priority to EP05447035.6, filed on Feb. 17, 2005, the contents of which are incorporated herein by reference in their entirety.

The present invention in particular relates to in vitro methods to identify and/or confirm the binding and/or function of a volatile compound onto a membrane-integrated receptor using a volatile-compound-Binding Protein (BP) or compositions thereof. Additionally, the present invention relates to kits comprising receptor proteins recognizing volatile compounds or a candidate receptor for said compound; and; BP, a complex or a composition thereof. Said kits may be used to identify and/or confirm the binding and/or function of volatile compounds onto a membrane-integrated receptor.

### BACKGROUND ART

Volatile compounds are small chemical entities which may be derived from any source, and when contained in a solid or liquid (either organic solvent or water) volatizes or evaporates at room temperature or an elevated temperature and, therefore, becomes present in the air or in discharge as vapor or smoke. Volatile organic compounds may include, but is not limited to, odorant molecules, which are part of the following fragrance families: aldehyde, fruity light, fruity dark, sweet aromatic, balsamic, aromatic spicy, tobacco, oakmoss, leather, animal, amber, woody, coniferous, herbal spicy, herbaceous, green, citrus. These odorant molecules also include but are not limited to chemical compounds bearing aldehydes, ketones, carboxylic acids, alkenes, ether oxide, phenols or alcohol groups. In addition to these odorant molecules, it is known that certain volatile compounds results in a taste, hormonal behavior and/or smell perception in mammals. Said compounds may help to orient cells and organisms such as sperm, animals and insects.

The sense of smell allows chemical communications between living organisms from invertebrates to mammals and environment. Perception and discrimination of thousands of odorants is made through olfaction. Such chemical signalling may modulate social behaviour of most animal species which rely on odorant compounds to identify kin or mate, to locate food or to recognize territory for instance. Smelling abilities are initially determined by neurons in the olfactory epithelium, the olfactory sensory neurons (OSN). Therein, odorant molecules bind to olfactory receptor proteins (OR), also known as odorant receptors. These OR are members of the G-protein coupled receptors (GPCR) superfamily. They are encoded by the largest gene family. While in rodents as many as 1300 different OR genes have been identified, around 800 OR genes have been identified in the human genome. Each olfactory neuron is thought to express only one type of OR, forming therefore cellular basis of odorant discrimination by olfactory neurons. They are synthesized in the endoplasmatic reticulum, transported and eventually concentrated at the cell surface membrane of the cilia at the tip of the dendrite. Similarly, ORs are found at the axon terminal of OSN. They are assumed to play a role in targeting axons to OR-specific olfactory bulb areas.

Most mammals have a secondary olfactory system, the vomeronasal system. The vomeronasal organ is localized in nasal cavity and is partly made of vomeronasal sensory neurons. This system would be responsible for detecting pheromones through activation of pheromone receptors. However, there is no evidence to affirm that detection of pheromone is solely done through vomeronasal sensory neurons and that vomeronasal sensory neurons detect pheromone only. Pheromone receptors are also 7TM proteins, but they are completely distinct from the OR superfamily. Even though pheromone receptors are part of the GPCR superfamily, no G-protein coupled to those receptors has been identified yet. Two families of pheromone receptors have been listed to date: the V1R and the V2R families. Receptors of both of them have been identified in mouse (more than 300) while only 5 receptors of the V1R family in human.

Taste is also part of chemosensation. It relies on the activation of taste receptors localized on the tongue and palate in human. They are expressed in taste receptor cells (TCRs) part of taste buds. These cells are specialized epithelium cells that contact neurons, which in turn relay the information to the brain. Thereby, unlike OSN, TCRs are not neuron cells. As olfactory receptors, taste receptors are part of the GPCR superfamily. Today, 2 families have been identified: T1R and T2R families. While human T1R family is made of three receptors namely T1R1, T1R2 and T1R3, T2R family is made of 25 putative receptors in human. T2R receptors are responsible for bitter taste detection and would be functional as monomers. However, T1R receptors are thought to work as dimmers. Dimerization would confer specificity to receptors. Heterodimers of T1R1/T1R3 detect umami taste, while T1R2/T1R3 heterodimers are activated by sweet compounds. Besides those two families, other proteins are thought to be taste receptors such as TRMP5, a potential channel, mGluR4 that might function as an umami receptor, ASIC2, a sour taste receptor, ENaC, a salt taste receptor, VN1, a burning taste receptor or TMP8, a cold taste receptor.

Smell, taste and pheromones constantly influence personal behaviour of animals and humans. It is thus of great importance to understand mechanisms of said perceptions. Most particularly to determine means to influence it. Already known is that olfactory, taste and pheromone systems do not follow the one ligand/one receptor rules. Several ligands have been described in the literature to activate same receptors. Therefore said sensory systems are probably part of a system wherein different receptors may be activated by same ligands, and wherein one receptor may be modulated by different ligands. In order to unravel said complex systems it is important to have sensitive methods which allow studying volatile-compound-binding receptors including OR, but also taste and pheromone receptors under physiological conditions that is in vivo-like conditions.

Some receptors have been described as candidate receptors whereon odorants, pheromones or taste-compounds may act. However, scientists are hampered by the fact that no methods are available to accurately study said receptors and/or ligands under physiological conditions. Indeed, current assays described in the literature include, but are not limited to, single cell calcium imaging and plate reader-based assays. In said assays concentrations of odorants up to 10⁻² molar are tested. Under these conditions, observed cellular responses are most likely non specific. Physiological concentrations are expected to lay around nano to femtomolar that is 10¹² less.

The large number of Olfactory Receptors (OR) necessitated setting up optimised methods, preferably high-throughput and high-sensitive screening methods, to deorphanising the all plethora.

Many proteins have been found to be present in olfactory mucus. One example is the Olfactory Binding Protein, also called OBP. OBP from many mammal species including bovine, rat, and porcupine OBP but also from many insect species are known to bind a wide variety of odorants with micromolar range affinity. The function of OBP in the olfaction process remains very controversial. In the one hand, because of its high concentration and its capacity to bind odorant molecules, OBP may possibly direct odorants from airway toward olfactory receptors (Pevsner et al. 1984. Proc. Natl. Acad. Sci. USA. 1985. 82:3050-3054; Pevsner et al. 1988. Science. 241:336-339; Pevsner et al. 1988. Proc. Natl. Acad. Sci. USA. 85:2383-2387; Avanzini et al. 1987. Cell. Tissue Res. 247:461-464; Lee et al. 1987. Science. 235:1053-1056; Bianchet et al. 1996. Nat. Struct. Biol. 3:934-939, Tegoni et al. 1996. Nat. Struct. Biol. 3:863-867, Pes et al. 1998. Gene. 212:49-55; Briand et al. 2002 Biochemistry. 41:7241-7252). On the other hand, there is evidence that in vivo OBP is a competitor to olfactory receptors and trap odorant molecules for further degradation fate (Boudjelal et al. 1996. Biochem. J. 317:23-27; Tegoni et al. 2000. Biochim. Blophys. Acta. 1482:229-240; Lazar et al. 2002. Biochemistry. 41:11786-117894). Since no functional evidence has been shown, deciphering these two hypotheses is not feasible. In some articles it is even suggested that OBP has both capacities depending of the concentrations of the odorant (Matarazzo et al. 2002 Chem. Senses 27:691-701). OBP are part of the Lipocalin proteins superfamily. These proteins are also characterized by the ability to form covalent or non covalent complexes with soluble macromolecules. Lipocalins are found in many fluids of mammals or insects.

Human Serum Albumin (HSA) is a high molecular weight endogenous plasma protein (MW 67 kDa). It is the main component of the blood transport system and provides the transport of fatty acids (FA), bilirubin, tryptophan, calcium and other physiologically important compounds. Different factors such as association with metabolites, toxins, pharmacological drugs are able to cause conformation modifications of the HSA molecule which can lead to transport malfunctions thereby to developments of some pathological processes. As HSA is part of the blood system, it has a broad role in the human body and is also present in most tissues. For instance, HSA is known to be preferentially accumulated by solid tumors. Also, Pernollet and collaborators have evidenced presence of Serum Albumin (SA) in the olfactory mucus. However, no specific function has been assigned to SA in olfactory process. Said serum albumin protein is found in species other than human including bovine specie (Bovine Serum Albumin, BSA).

### DETAILED DESCRIPTION OF THE INVENTION

As mentioned above, because they require high concentrations of odorants, current methods used to study ligand/receptor interaction most likely lead to non-specific cellular responses. In fact, as most volatile compounds are hydrophobic they probably form micelles at high concentration altering their efficacy and specificity, as already described by McGovern et al. (J. Med. Chem. 2002. 45:1712-1722 and J. Med. Chem. 2003. 46: 4265-4272). Furthermore, said methods are hampered as the trafficking of said volatile-compound-binding receptors to the cell membrane is inefficient, making the characterization and/or study of said receptors difficult. Therefore at this moment only few volatile-compound-binding receptors including OR could be studied and their ligand identified. Consequently, there is an urge to optimise existing methods enabling studying interaction and effect of volatile compounds, also called volatile ligands, with/on their receptors.

The present invention is directed towards providing novel methods, which allows the study of interaction of volatile compounds with their receptors under conditions much closer to the known physiological conditions than currently used. Said method may be cell-based or membrane-based method.

Optimisation of prior art methods may be found in further solubilizing volatile compounds to form a complex with a volatile-compound-binding protein (BP). With the term “volatile-compound-binding protein” is meant any protein that bind volatile compound(s). According to the present invention said BP may be Lipocalin or Serum Albumin (SA). According to the invention, Lipocalin may be chosen from the group consisting of odorant binding protein (OBP), pheromone binding protein (PBP), Retinol binding protein (RBP), major urinary protein (MUP), aphrodisin and von Ebner gland protein. Said Lipocalins and Serum albumin may originate from any species. For instance said Lipocalin may originate from mouse, rat, human, bovine, pig, porcupine and rabbit; said SA may be chosen from the group consisting of human serum albumin (HSA), bovine serum albumin (BSA), boar serum albumin, rabbit serum albumin, mouse serum albumin, pig serum albumin and rat serum albumin. A skilled person may easily determine if a protein is capable of binding a volatile compound. Examples of such tests are given in Example 12. That a protein is a member of the Lipocalin or the serum albumin family may be determined as explained in Examples 13 and 14.

Said BP-complex is then used to modulate volatile-binding-compound receptors activity, including OR, which are known, or are candidate, to recognize said volatile compound.

As mentioned above, one of the drawbacks of the currently running methods lays in the fact that most volatile compounds, such as odorant molecules, are hydrophobic forming micelles in an aqueous solution thereby leading most likely to non specific cellular responses. The present invention found unexpectedly that volatile-compound-binding proteins (BP), which are specific carrier molecules including Serum Albumin (SA) and Odorant Binding Protein (OBP), improve solubilization and presentation of volatile ligand to its receptor. Consequently, the present invention suggests using said carrier molecules to set up improved methods to study ligand/receptor interaction of volatile-compound-binding receptors. According to the present invention, said carrier proteins may be present in a composition such as natural occurring mucus, e.g. the mucus of olfactory cavity, or fractions thereof, or may be present as a more purified proteins or compositions thereof. In particular, the present invention experimentally proves that BP play a critical role as carrier in the solubilization and presentation of odorants to their receptor. However, as receptors recognizing volatile-compounds other than odorants, such as pheromone receptors and taste receptors, behave similarly, and as the ligands for said receptors have similar chemical properties, the invention suggests that the optimisation of the methods illustrated for odorant receptors may be applied to optimise methods wherein ligand-receptor interactions of other volatile-compound-binding receptors are studied. While BP protein for taste receptors would be von Ebner gland proteins as well as OBP, Pheromone Binding Protein (PBP) would be carrier of pheromone.

It has been shown in literature that Odorant Binding Protein (OBP) may bind odorants. However, it has also been shown that Olfactory Receptor (OR) activation by an odorant is not dependent upon the presence of OBP, suggesting that the role of said OBP relates more as regulator molecule to control the signalling induced by said odorant. Until now, nobody tried to study the effect of OBP on the volatile-ligand solubilization and presentation to its receptor. The present invention found that the role of OBP lays in a better solubilization and presentation of the odorant ligand to the receptor. As said role is positive, the present invention suggests that the use of said OBP, or a composition thereof, may be used to improve in vitro methods to identify and characterize ligand/receptor interactions for olfactory receptors. Using a novel olfactory functional assay based on the peri-receptor event, the present invention illustrates that bovine olfactory mucus can capture odorant molecules and enhance their efficacy to activate olfactory receptors. The present invention further shows that fractions of this mucus containing OBP and SA play the role of carrier protein. Finally, the present invention demonstrates that a solution of bovine Serum Albumin alone can play the role of odorant carrier protein.

The present invention thus in particular relates to the finding that Serum Albumin (SA) and more generally a volatile-compound-Binding Protein (BP) may be used to optimise binding conditions for volatile-compounds to bind their receptors in in vitro assays. The present invention suggests that SA and more generally BP make volatile compounds more soluble, making complexes which may be applied in functional and binding assays for volatile-compound binding receptors.

The present invention relates to methods which may be applied to study different kinds of volatile-compound-binding receptors, such as olfactory receptors, taste receptors and pheromone receptors. Consequently, the methods of the present invention may be used for any industry including food industry, health industry, cosmetic industry, militaries, sanitary agencies, animal sniffers (e.g. for drugs, explosives, accident victims etc.) among many others. For example, the present invention provides a systematic way to identify which receptors and ligands are responsible for particular olfactory sensations (e.g. perceived scents). Thus for example, by blocking particular reactions (e.g. via nasal spray having the inhibitors) or enhancing particular interactions (e.g. via a nasal spray that provide certain ligands or a coating on the surface of an object that emits certain ligands) one can control perceived scents. Thus, undesired scents can be blocked, covered, or altered (e.g. a snifferdog can be treated so as to only smell a target of interest and no other distracting smells, a sanitary worker can be made immune to the scent of waste, etc.) and desired scents can be enhanced. It is also clear that possibilities for modulating taste and pheromones will find a lot of applications in many aspects. In the paragraphs below the methods of the present invention are elaborated.

A first embodiment of the present invention relates to a method to identify and/or to confirm the binding and function of a volatile compound onto a membrane-integrated receptor. Said method may comprise the steps of:
selecting a compound which may be a ligand for said receptor,
solubilizing said compound by airborne absorption onto a volatile-compound-Binding Protein (BP), making a BP-ligand-complex,
applying said ligand complex on cells expressing said receptor,
measuring the functional response of said receptor, and,
identifying a ligand for said receptor and/or confirming the binding and function of said compound onto said receptor.

A second embodiment of the present invention relates to a method to identify and/or confirm the binding of a volatile compound onto a membrane-integrated receptor. Said method may comprise the steps of:
selecting a compound which may be a ligand for said receptor,
solubilizing said compound by airborne absorption onto volatile-compound-Binding Protein (BP), making a BP-ligand-complex,
applying said ligand complex to cell membranes comprising said receptor,
measuring the binding of said compound to said receptor, and,
identifying a ligand for said receptor and/or confirming the binding of said compound onto said receptor.

According to the present invention, the above-mentioned methods may be used to deorphanise volatile compounds and/or deorphanise receptors recognizing volatile-compounds. This may help to unravel the complex system wherein a volatile compound may recognize different receptors, and wherein a receptor is recognized by different volatile compounds. Complex assays may thus be set up using one volatile-compound-binding receptor with the aim to identify one, or a set of volatile compound(s) which bind(s) specifically to said receptor. Once (a) volatile ligand(s) is/are identified, one of these ligands may be used to screen a panel of different receptors which are known or candidate to recognize volatile compounds. Alternatively, complex assays may be set up to first screen a ligand onto different (candidate) volatile-compound binding receptors. Once (a) receptor(s) is/are identified as binding to said ligand, one of these receptors may be used to screen a panel of different volatile compounds. In both approaches the specificity/preference of said ligand/receptor interaction may be determined.

Different subtypes of BP can be found in one species. It has been suggested in literature that said different BPs may have different ligand-specificity. If so, said specificity still needs to be elucidated. Therefore, the methods of the present invention may also be used to identify the specificity of a BP for a (group of) volatile ligand(s) and its/their effect on a specific receptor.

Although it is suggested that SA and Lipocalins may have a similar effect as carrier on the ligand/receptor binding interaction of volatile-compound-binding receptors, the present invention does not exclude that both molecules may work cooperatively as a complex or subsequently.

While in the binding assay described in the present invention, binding of compound(s) onto receptor(s) is measured; in the functional assay, not only binding is measured but also capacity of a molecule to modulate positively of negatively the tested receptor. Compounds which positively modulate the tested receptor, thereby resulting in activation of the signaling pathway are called (full or partial) agonists for said receptor. Compounds which negatively modulate the tested receptor, thereby resulting in inhibition of the signaling pathway, are called (full or partial) antagonists or inverse agonists for said receptor. A compound may behave competitive or noncompetitive for another compound, depending if it can displace said latter compound from the receptor or not.

The term ‘volatile molecule’ or ‘volatile compound’ or ‘volatile agent’ or ‘volatile ligand’ refers to any chemical volatile entity. Said compound may be obtained using any of the numerous approaches in combinatorial library methods known in the art, including biological libraries, synthetic chemical library methods requiring deconvolution, and the ‘one compound’ chemical library method.

Testing the ability of a volatile compound to bind a receptor can be performed using different approaches. This can be accomplished, for instance, by coupling the compound with a radioisotope such that binding of the compound can be determined by detecting the labeled compound in a complex. For example, compounds may be labeled with ¹²⁵I, ³⁵S, ¹⁴C or ³H, either directly or indirectly, and the radioisotope detected by direct counting of radioemmission or by scintillation counting. A difference in the binding of different tested compounds may indicate which compound recognizes more efficiently a receptor compared to others. Alternatively, the binding of said volatile compounds to the receptor may be measured in the presence of a reference compound. The binding of the compound to its receptor may comprise a step of contacting the said receptor with a reference volatile-compound complexed with the volatile-compound-Binding Protein in the presence and in the absence of a candidate modulator also present in such complex under conditions permitting the binding of said complex and/or candidate alone to said receptor, and, a step of measuring the binding of said volatile compound-complex and/or candidate alone to said receptor, wherein a decrease in binding in the presence of said candidate modulator, relative to the binding in the absence of said candidate modulator, identifies said candidate modulator as an agent that modulates the function of said receptor. In said case the reference compound is labelled. Such volatile compounds may be part of the following fragrance families: aldehyde, fruity light, fruity dark, sweet aromatic, balsamic, aromatic spicy, tobacco, oakmoss, leather, animal, amber, woody, coniferous, herbal spicy, herbaceous, green, citrus. These odorant molecules also include but are not limited to chemical compounds bearing aldehydes, ketones, carboxylic acids, alkenes, ether oxide, phenols or alcohol groups. Alternatively, one of the carrier molecules may be labelled.

Another approach is to determine functional modulation of a receptor by a volatile-compound. This may be performed in the presence or in the absence of a reference compound. Modulation of said receptor by said compound may comprise the following steps 1/contacting a receptor with a reference compound complexed with a volatile-compound-Binding Protein in the presence and in the absence of a candidate modulator also present in such complex; 2/ measuring a signalling activity of said receptor, wherein a change in the activity in the presence of said candidate modulator relative to the activity in the absence of said candidate modulator identifies said candidate modulator as an agent that modulates the function of said receptor. Using this method, the finding of an antagonist, agonist or inverse-agonist is aimed at. Alternatively, the functional modulation of the receptor by a volatile-compound may comprise the steps of contacting the receptor with a candidate modulator-BP complex; measuring a signalling activity of said receptor in the presence of said candidate modulator; and comparing said activity measured in the presence of said candidate modulator to said activity measured in a sample in which said volatile-compound-binding receptor is contacted with a reference volatile-compound at its EC₅₀, wherein said candidate modulator is identified as an agent that modulates the function of said receptor when the amount of said activity measured in the presence of said candidate modulator is at least 50% of the amount induced by said reference compound present at its EC₅₀. The aim for both the binding and the functional methods may be the identification of a volatile agent that binds and/or modulates a receptor. When no reference molecules are available, the method of the present invention may be used to de-orphanize the volatile-compound-binding receptor.

Alternatively, functional responses may also be studied on cell membranes e.g. through the determination of GTPγS binding. In general, for binding assays of the present invention cell membranes are mostly applied, while intact cells are used for functional assays.

Prior to the filing of the present application, it was not clear how a volatile-compound-Binding Protein may influence the binding and/or modulation of volatile-compounds to their receptor(s). For instance it was suggested by Matarazzo et al (2002, Chem. Senses 27: 691-701) that at low odorant concentration, the OBP may favor the uptake of the odorant in the mucus layer (p. 699, sec column, I. 12-15); however, when the ligand concentration is high, it would prevent saturation of the OR binding sites. As OBP is also suggested to play a role of scavenger, said molecule would never been used as a carrier molecule in assays wherein binding of ligands to its receptor, also at higher concentrations is measured. Contrarily, according to the concept of the present invention, said odorant may also be used at higher concentrations without interfering with the ligand-activation of the receptor. Based on said information, a method may be set up to determine ligand binding in a dose-response-like fashion. It has also been shown in literature that OR may be activated by odorant, independent upon the presence of OBP. The present invention shows thus for the first time that ligand-OBP-OR, ligand-SA-OR or ligand-OBP/SA-OR complex formation is of general physiological relevance for OR modulation and may be applied in in vitro methods to study volatile-compound-binding receptor. The present invention also demonstrates via single-cell-Ca²⁺-imaging that when using a method of the present invention, a signal can be measured in nearly all tested cells, rather than a small fraction as usually described in the literature. This indicates that the methods of the present invention may be easily applied in small volume screening methods, in particular high throughput screening methods (see below).

Serum Albumin is a protein which is commonly present in different parts of the body. This protein may be found in most tissues as it forms a major compound of the blood, and the blood flow most tissues. Pernollet and collaborators have found that serum albumin is present in the olfactory mucus. However, no olfactory function has been assigned to it yet. The present invention presents Serum Albumin as alternative molecule to Lipocalin. The present invention suggests that SA behaves as a carrier protein, which solubilizes and presents volatile compound to volatile-compound-binding receptors, including OR.

The present invention gives evidence that OBP and SA work to make volatile-compound more soluble and only work as carrier molecule and not as (partly) scavenger as suggested before for OBP. Therefore, said molecules may be used in ligand-binding and functional assays. In the present invention experimental evidences for this are given through detection of OR activation by odorants. However, the role of OBP and SA towards the ligand/receptor recognition is not ligand and/or receptor specific. Therefore, the concept of the present invention may be generalized for other volatile-ligands and for other receptors recognizing volatile-compounds. Consequently, as the methods are not limited to a specific kind of volatile-compound binding receptor; said membrane integrated receptor may be chosen from the group consisting of, but not limited to, a G Protein Coupled Receptor, an ion-channel, and a Tyrosine kinase receptor; recognizing a volatile compound. According to the present invention, the membrane integrated receptor may be chosen from the group consisting of an olfactory receptor, a pheromone receptor, a taste receptor, and, any kind of membrane-bound receptor recognizing a volatile-compound. As used herein the term ‘odorant receptor’ refers to odorant receptors generated from olfactory sensory neurons. Examples of olfactory receptors which may be studied according to the methods of the present invention may be, but are not limited to, I7, M71, MOR23, mOR-EG, mOR-EV, U131, I-C6, I-D3, I-G7, mOR912-93, OR17-40, OR174. Human OR which have been identified so far and may be used in the methods of the present invention are published in Malnic et al. (PNAS. 2004. 101 (8): 2584-2589). Said receptors are hereby included by reference. Examples of pheromone receptors may be, but are not limited to, hV1RL1, hV1RL2, hV1RL3, hV1RL4, hV1RL5, V1R and V2R. Pheromone receptors which have been identified so far and may used in the methods of the present invention are published in Rodriguez (Horm. Behav, 2004. 46 (3):219-230) and Matsunami and Amrein (Genome Biol, 2003. 4 (7):220). Said receptors are hereby included by reference. Examples of taste receptors may be, but are not limited to, T2R, T1R1, T1R2, T1R3, ASIC2, ENaC, VN1 and TMP8. Taste receptors which have been identified so far and may used in the methods of the present invention are published in Scott (Curr. Opin. Neurobiol. 2004. 14 (4):423-7); Clafani A (Appetite 2004. 43 (1):1-3), Huang (J. Am. Soc. Nephrol. 2004. 15 (7): 1690-9), Kim et al. (J. Dent. Res. 2004. 83 (6):448-53), Ugawa (Anat. Sci. Int. 2003. 78 (4):205-10), Bigiani et al. (Prog. Biophys. Mol. Biol. 2003. 83 (3): 193-225) and Matsunami and Amrein (Genome Biol. 2003. 4 (7):220). Said receptors are hereby included by reference.

In the method of the present invention, wherein the volatile-compound-Binding Protein (BP) may be of mammalian or insect origin.

Furthermore, according to the present invention the volatile-compound-Binding Protein (BP) used may be chosen from the group consisting of Lipocalin, serum albumin (SA) and any protein having the capacity of binding a volatile compound and functioning as a carrier to present said volatile compound to membrane-integrated receptors. Said Lipocalin may be chosen from the group consisting of Olfactory Binding Protein (OBP), Pheromone Binding Protein (PBP), retinol binding protein (RBP), major urinary protein (MUP), aphrodisin and von Ebner gland protein.

As used herein the term Olfactory Binding Protein (OBP), encompasses proteins that are identical to wild-type OBP and those that are defined from wild type OBP (e.g. variants of OBP) or chimeric proteins constructed with portions of OBP coding regions. In the sections below sequences are listed referring to particular polypeptides. Nucleic acid sequences coding for said particular polypeptides are also mentioned as they can form part of a kit of the present invention (see below).

In some embodiments the OBP is a wild type murine OBP nucleic acid (mRNA) (sequence 1 of Table 1) or polypeptide encoded by the wild type murine OBP nucleic acid sequence (sequence 2 of Table 1). In other embodiments the OBP is a wild type rat OBP nucleic acid (mRNA) (sequence 3, 4 and 5 of table 1) or polypeptide encoded by the wild type rat OBP nucleic acid sequence (sequence 6, 7 and 8 of Table 1). In other embodiments, the OBP is a wild type human OBP nucleic acid (mRNA) (sequence 9, 10 and 11 of Table 1) or polypeptide encoded by the wild type human OBP nucleic acid sequence (sequence 12, 13 and 14 of Table 1). In other embodiments, the OBP is a wild type bovine OBP nucleic acid (mRNA) or polypeptide encoded by the wild type bovine OBP nucleic acid sequence (sequence 15 of Table 1). In other embodiments the OBP is a wild type pig OBP nucleic acid (mRNA) (sequence 16 of Table 1) or polypeptide encoded by the wild type pig OBP nucleic add sequence (sequence 17 of Table 1). In other embodiments the OBP is a wild type rabbit OBP nucleic acid (mRNA) or polypeptide encoded by the wild type rabbit OBP nucleic acid sequence. In other embodiments the OBP is a wild type porcupine OBP nucleic acid (mRNA) or polypeptide encoded by the wild type porcupine OBP nucleic acid sequence. However, the present invention does not rule out the OBP of other species. For instance insects are known to synthesize lot of OBP or PBP (Pheromone Binding Protein) in their antenna.

As used herein the term Serum Albumin (SA), encompasses both proteins that are identical to wild-type SA and those that are defined from wild type SA (e.g. variants of SA) or chimeric polypeptide constructed with portions of SA coding regions. In some embodiments the SA is, but is not limited to, a wild type bovine SA nucleic add (mRNA) (sequence 18 of Table 1) or polypeptide encoded by the wild type SA nucleic acid sequence (sequence 19 of Table 1). In other embodiments the SA is, but is not limited to, a wild type human SA nucleic add (mRNA) (sequence 20 of Table 1) or polypeptide encoded by the wild type human SA nucleic add sequence (sequence 21 of Table 1). In other embodiments the SA is but is not limited to a wild type pig SA nucleic acid (mRNA) (sequence 22 of Table 1) or polypeptide encoded by the wild type pig SA nucleic acid sequence (sequence 23 of Table 1). In other embodiments the SA is but is not limited to a wild type rabbit SA nucleic acid (mRNA) (sequence 24 of Table 1) or polypeptide encoded by the wild type rabbit SA nucleic add sequence (sequence 25 of Table 1). In other embodiments the SA is but is not limited to a wild type mouse SA nucleic add (mRNA) (sequence 26 of Table 1) or polypeptide encoded by the wild type mouse SA nucleic add sequence (sequence 27 of Table 1). In other embodiments the SA is but is not limited to a wild type rat SA nucleic acid (mRNA) (sequence 28 of Table 1) or polypeptide encoded by the wild type rat SA nucleic acid sequence (sequence 29 of Table 1).

As used herein the term ‘von Ebner gland protein’ encompasses both proteins that are identical to wild-type protein and those that are defined from said wild type protein (e.g. variants) or chimeric polypeptide constructed with portions of coding regions of said protein. In some embodiments the von Ebner gland protein is, but is not limited to, a wild type bovine von Ebner gland protein nucleic acid (mRNA) (sequence 30 of Table 1) or polypeptide encoded by the wild type von Ebner gland protein nucleic acid sequence (sequence 31 of Table 1). In other embodiments the von Ebner gland protein is, but is not limited to, a wild type wild boar von Ebner gland protein nucleic acid (mRNA) (sequence 32 of Table 1) or polypeptide encoded by the wild type wild boar von Ebner gland protein nucleic acid sequence (sequence 33 of Table 1). In other embodiments the von Ebner gland protein is, but is not limited to, a wild type human von Ebner gland protein nucleic acid (mRNA) (sequence 34 of Table 1) or polypeptide encoded by the wild type human von Ebner gland protein nucleic acid sequence (sequence 35 of Table 1).

As used herein the term Major Urinary Protein (MUP) encompasses both proteins that are identical to wild-type MUP protein and those that are defined from said wild type protein (e.g. variants) or chimeric polypeptide constructed with portions of coding regions of said protein. In some embodiments the MUP protein is, but is not limited to, a wild type mouse MUP nucleic acid (mRNA) (sequence 36 of Table 1) or polypeptide encoded by the wild type mouse MUP nucleic acid sequence (sequence 37 of Table 1).

As used herein the term Pheromone Binding protein (PBP) encompasses both proteins that are identical to wild-type PBP protein and those that are defined from said wild type protein (e.g. variants) or chimeric polypeptide constructed with portions of coding regions of said protein. In some embodiments the PBP is, but is not limited to, a wild type Helicoverpa assulta PBP nucleic acid (mRNA) (sequence 38 of Table 1) or polypeptide encoded by the wild type Helicoverpa assulta PBP nucleic acid sequence (sequence 39 of Table 1). In other embodiments the PBP is, but is not limited to, a wild type Sesamia nonagrioides PBP1 nucleic acid (mRNA) (sequence 40 of Table 1) or polypeptide encoded by the wild type Sesamia nonagrioldes PBP1 nucleic acid sequence (sequence 41 of Table 1). In other embodiments the PBP is, but is not limited to, a wild type Sesamia nonagrioides PBP2 nucleic acid (mRNA) (sequence 42 of Table 1) or polypeptide encoded by the wild type Sesamia nonagrioides PBP2 nucleic acid sequence (sequence 43 of Table 1). In other embodiments the PBP is, but is not limited to, a wild type Spodoptera PBP1 nucleic add (mRNA) (sequence 44 of Table 1) or polypeptide encoded by the wild type Spodoptera PBP1 nucleic acid sequence (sequence 45 of Table 1). In other embodiments the PBP is, but is not limited to, a wild type Spodoptera PBP2 nucleic acid (mRNA) (sequence 46 of Table 1) or polypeptide encoded by the wild type Spodoptera PBP2 nucleic acid sequence (sequence 47 of Table 1). In other embodiments the PBP is, but is not limited to, a wild type Drosophila PBP nucleic acid (mRNA) (sequences 48, 50, 52, 54 and 56 of Table 1) or polypeptide encoded by the wild type Drosophila PBP nucleic acid sequence (sequence 49, 51, 53, 55 and 57 of Table 1).

According to the present invention, the volatile-compound-Binding Protein may be a wild type protein or a variant protein. With the term ‘wild type’ is meant naturally occurring types or subtypes. ‘Wild type’ and ‘variant’ may refer to nucleic acid or amino acid sequences. Said variant or mutant nucleic acid or amino acid may be 70%, preferably 80%, more preferably 90%, and most preferably 95% homologous to the nucleic add sequences or amino acid sequences mentioned above, as long as said carrier molecule has a positive effect on the transfer of volatile ligand to its receptor. Variant forms of BP polypeptides are also contemplated as being equivalent to those peptides and DNA molecules that are set forth in more detail herein. For example, it is contemplated that isolated replacement of a leucine residue with an isoleucine or valine residues, an aspartate residue with glutamate residue, a threonine residue with a serine residue, or a similar replacement of an amino acid with a structurally related amino add (i.e. conservative mutations) will not have a major effect on the biological activity of the resulting molecule. Accordingly, some embodiments of the present invention provide the use of variants of BP containing conservative replacements. Conservative replacements are those that can take place within a family of amino adds that are related in their side chain. Genetically encoded amino acids can be divided into four families: (1) acidic (aspartate and glutamate residues); basic (lysine, arginine, and histidine residues); (3) nonpolar (glycine, alanine, valine, leucine, isoleucine, proline, phenylalanine, methionine, and thryptophan residues); and (4) uncharged polar (asparagine, glutamine, cysteine, serine, threonine, and tyrosine residues). Phenyl alanine, thryptophan and tyrosine residues are sometimes classified jointly as aromatic amino acids. In similar fashion, the amino acid repertoire can be grouped as (1) acidic (aspartate and glutamate residues); basic (lysine, arginine, and histidine residues), (3) aliphatic (glycine, alanine, valine, leucine, isoleucine, serine, and threonine residues), with serine and threonine optionally be grouped separately as aliphatic-hydroxyl; (4) nonpolar (phenylalanine, tyrosine, and tryptophan residues); (5) amide (asparagine and glutamine residues); and (6) sulfur-containing residues (cysteine and methionine residues) (e.g. Stryer ed. Biochemistry, pg. 17-21, 2^{nd} ed, WH Freeman and Co., 1981). Whether a change in the amino acid sequence of a peptide results in a functional polypeptide can be readily determined by assessing the ability of the variant to function in a fashion similar to the wild-type protein. Polypeptides having more than one replacement can readily be tested in same manner. More rarely, a variant may include ‘non-conservative’ changes (e.g., replacement of a glycine with a tryptophan residue). Analogous minor variations can also include amino acid deletions or insertions, or both. Guidance in determining which amino acid residues can be substituted, inserted, or deleted without abolishing biological activity can be found using computer programs (e.g. LASERGENE software, DNASTAR Inc., Madison Wis.). As described in more detail below, variants may be produced by methods such as directed evolution or other techniques for producing combinatorial libraries of variants. In still other embodiments of the present invention, the nucleotide sequences coding for BP may be engineered in order to alter said sequence, including, but not limited to, alternations that modify the cloning, processing, localization, secretion, and/or expression of the gene product. For example, mutations may be introduced using techniques that are well known in the art. Those techniques include, but are not limited to, site directed mutagenesis to insert new restriction sites, alteration of glycosylation patterns, or change of codon preference.

The term ‘variant proteins’ not only refers to a (possibly engineered) protein comprising small AA variants compared to the naturally occurring protein sequences, but also referring to variants comprising larger modifications such as fusion proteins or fragments thereof. For instance, the domain called “calix” (binding pocket) may be used for this purpose. Said calix is an assembly of sequences issued from the tertiary structure of the considered protein. It is known for a skilled person that the calix domain may differ from protein to protein. Based on said tertiary structure, a skilled person may derive for each protein the calix sequence. For instance, it has been shown that OBP that dimerize would have one binding pocket as OBP-1F (Nespoulous C, Briand L, Delage M M, Tran V and Pernollet J C 2004. Odorant binding and conformational changes of a rat odorant binding protein. Chem. Senses 29 (3): 189-98), two or even three binding pockets one in each barrel and another one at the intersection of the two barrels (Tegoni M, Ramoni R., Bignetti E, Spinelli S and Cambllau C 1996. Domain swapping create a third putative combining site in bovine odorant binding protein dimmer. Nature Struct. Biol. 3: 934-9. In addition, the calix sequence of Bovine lactaglobulin is known (Qin et al. 1998. FEBS Lett. 438 (3):272-8). In addition, the calix may be taken out OBP to be placed in other scaffold protein to engineer an “OBP-like protein”. Said volatile-compound-Binding Protein may be from any origin, purified or part of an extract.

The present invention relates to a method to study in vitro a volatile-compound-binding receptor in the presence of a ligand complexed with BP. In said method the receptor and BP (including SA and Lipocalin) used may be of the same origin (i.e human or bovine), however the present invention does not exclude study of a receptor of a particular origin (i.e. human) with carrier proteins of another origin (i.e. bovine). When using both SA and Lipocalin as carrier protein, also the origin of said carriers may be different indeed as Lipocalins of different origins are similar in structure, their effects in the methods proposed are expected to be the same.

BP, in particular Lipocalins or SA, used in a method of the present invention may be purified according to methods known in the art. Said purification methods include, but are not limited to, ammonium sulfate or ethanol precipitation, add extraction, anion or cation exchange chromatography, gel filtration chromatography, phosphocellulose chromatography, hydrophobic interaction chromatography, affinity chromatography, hydroxylapatite chromatography and lectin chromatography. In other embodiments protein-refolding steps can be used as necessary, in completing configuration of the mature protein. In still other embodiments, high performance liquid chromatography (HPLC) can be employed for final purification steps. The nucleotides coding for BP, including SA and Lipocalin, may be fused in frame to a marker sequence that allows purification of BP polypeptides. A non-limiting example of a marker sequence is a hexahistidine tag which may be supplied by a vector, which provides for purification of the polypeptide fused to the marker in the case of a bacteria host for expression, or for example, the marker sequence may be a hemagglutinin (HA) tag when a mammalian host for expression (e.g., COS-7 cells) is used. The HA tag corresponds to an epitope derived from the influenza hemagglutinin protein (Wilson et al., Cell 37:767 (1984)). In addition, in the methods of the present invention fragments of BP, or fusions comprising the full length or fragments thereof, may be used which still carry a carrier property as found in the wild type protein.

As mentioned above, volatile-compound-Binding Protein may be purified. Alternatively said proteins may be present in a composition. According to the present invention, said composition may be a natural fluid or fractions thereof. With natural fluids is meant all fluids possible secreted from cells (individually cells, or glands or tissues from the animal body including blood). Said fluids may be for instance nasal, respiratory, salivary (von Ebner gland protein), urinary (major urinary protein, MUP), liver (MUP) and vaginal secretion (aphrodisin), but may also comprise fluids wherein BP can be found. With ‘fraction’ is meant, part of a composition which comprises a molecule of interest.

According to the present invention, the volatile-compound-Binding Protein (BP) used in the methods described above may be a member of the Lipocalin family wherein said proteins have a canonical super-secondary structure. It has been previously shown that said specific structure allows the binding of odorants. Other volatile compounds most likely bind Lipocalin and Lipocalin-like protein.

In an alternate embodiment of the invention used BP polypeptides may be produced using chemical methods to synthesize either a full length BP amino add sequence or a portion thereof. For example, peptides can be synthesized by solid phase techniques, cleaved from the resin, and purified by preparative high performance liquid chromatography (see e.g., Creighton, Proteins Structures and Molecular Principles, W. H. Freeman and co, New York N.Y. (1983)). In other embodiments, the composition of the synthetic peptides is confirmed by amino acid analysis or sequencing (See e.g., Creighton, supra). Direct peptide synthesis can be performed using various solid-phase techniques and automated synthesis may be achieved, for example, using ABI 431A Peptide Synthesizer (Perkin Elmer) in accordance with instructions provided by the manufacturers. Additionally, the amino acid sequence of a BP may be altered direct synthesis and/or combined using chemical methods with other sequences to produce a variant polypeptide (see above).

According to the present invention, the volatile-compound-Binding Proteins may function as carrier molecule as a monomer, a homodimer or heterodimer, a homomultimer or heteromultimer thereof. It is for instance known that hOBP may exist as a monomer at neutral pH like several OBP, while others are known as dimmers such as rat, pig and bovine OBP. As thus the complexity of the BP ligand complex formed when following the method of the present invention, is not completely understood, we may not exclude that said carrier proteins may work as mono-, di- or multimers. OBP belongs to the Lipocalin family, forming a typical Lipocalin binding pocket. It was previously suggested that odorants enter the β-barrel pocket of OBP with their hydrophobic moiety inside (Briand et al. 2002). It was also previously suggested that a set of complementary OBP with different specificity would be necessary to solubilize a vast array of diverse odorants, which are perceived. For porcupine OBP monomer-dimer equilibrium seems to depend on the experimental conditions.

In the method of the present invention, the functional response may be measured by studying cellular signaling molecules chosen from the group: Ca2+, cAMP pool, IP3, GTP, melanophore assay and MAP-kinase; or, wherein the functional response may be measured by studying G protein/OR interaction, desensitization of the receptor, or, wherein the functional response may be measured by studying the modulation of a reporting system. The invention contemplates the use of natural cell lines or heterologous cell lines transfected with a volatile-compound binding receptor or variants thereof for screening compounds for activity, and in particular to high throughput screening of compounds from combinatorial chemical libraries (e.g., libraries containing greater than 10⁴ compounds). In some embodiments, the cells can be used in second messenger assays that monitor signal transduction following activation of cell-surface receptors. In other embodiments the cells can be used in reporter gene assays that monitor cellular responses at the transcription/translation level. In second messenger assays, the host cells are preferably transfected with vectors encoding a receptor, or a candidate therefore, recognizing a volatile compound. The host cells may then be treated with a ligand-BP complex or a plurality of said complexes (e.g. from a combinatorial library) and assayed for the presence or absence of a response. It is contemplated that at least some of the compounds in the combinatorial library can serve as agonists, antagonists, activators or inhibitors of the volatile-compound binding receptors at the cell membrane.

In some embodiments, the second messenger assays measure fluorescent signals triggered by receptor molecules activation that in turn lead to intracellular changes (e.g. Ca2+ concentration, membrane potential, pH, IP3, cAMP, arachidonic acid release) due to stimulation of membrane receptors and ion channels. Examples of reporter molecules include, but are not limited to, FRET (fluorescence resonance energy transfer) systems (e.g., Cuo-lipids and oxonols, EDAN/DABCYL), calcium sensitive indicators (e.g., Fluo-3, FURA2, INDO1 and FLUO4/AM, BAPTA AM, Calcium3), chloride-sensitive indicators (e.g., SPQ, SPA), potassium-sensitive indicators (e.g., PBFI), sodium-sensitive indicators (e.g., SBFI), and pH sensitive indicators (e.g., BCECF). In general the host cells are loaded with the indicator prior to exposure to compounds. Responses of the host cells to treatment with volatile compound-BP complex can be detected by methods known in the art, including, but not limited to fluorescence microscopy, confocal microscopy (e.g., FCS systems), flow cytometry, microfluidic devices, FLIPR systems and plate reading systems. The present invention also notes that other methods may be used for this purpose including, but not limited to, gene-reporter system (including Luciferase systems), GTPγS assay, G protein/OR interaction-based assays by for instance FRET or BRET assays, or assays studying receptor desensitization including β2-arrestin assay.

As mentioned in some of the paragraphs above, the response (e.g., increase in fluorescent intensity) caused by the compound of unknown activity is preferably compared to the response generated by a known agonist and expressed as a percentage of the maximal response of the known agonist. The maximum response caused by a known agonist is defined as a 100% response. Likewise, the maximal response recorded after addition of an agonist to a sample containing a known or test antagonist is detectably lower than the 100% response.

The methods of the present invention may be set up as a reporter system. With the term ‘reporter system’ is meant a system that applies gene encoding a protein that may be assayed. Examples of reporter genes include, but are not limited to, luciferase, green fluorescent protein, chloramphenicol acetyltransferase, b-galactosidase, alkaline phosphatase and horse radish peroxidase. Reporter gene assays preferably involve the use of host cells transfected with vectors encoding a nucleic acid comprising transcriptional control elements of a target gene (i.e. a gene that controls the biological expression and function of a disease target) spliced to a coding sequence for a reporter gene. Therefore activation of the target gene results in the activation of the reporter gene product. Alternative reporting systems which may be used are for instance the CRE-luciferase assay, melanophore assay, or the MAP-kinase assay. The CRE-luciferase assay is based on following principle: when the membrane receptor becomes activated, the cellular cAMP pool increases; said cAMP may bind to a CRE (cAMP responsive element) linked to a gene coding for a reporter protein (in this case luciferase). Receptor activation can thus easily be measured through the appearance of a luminescent signal. As the volatile-compound-binding receptor may stimulate different signaling molecules, different mechanisms may be used to set up a similar assay. Responsive elements to certain secondary messengers may thus be linked to a marker gene.

The melanophore assay is a color-based assay. Basically cells used for this assay are derived from skin of the frog Xenopus Laevis. These immortalized cells contained melanosomes, which are organelles containing dark pigment. Activation of endogenous or recombinant GPCR that trigger activation of adenylate cyclase or phospholipase C lead to melanosome dispersion and thereof cell darkening. Alternatively, GPCR that inhibits adenylate cyclase or phospholipase C leads to cell lightening. Thereby, instead of measuring concentrations of second messenger, one can easily pinpoint hit observing cell coloration change. This color change can easily be quantified on a microplate reader measuring absorbance at 650 nM or by examination on a video imaging system.

The ability of a volatile compound, present in a BP complex, to interact with a receptor can also be evaluated without labeling any of the interactants. For example, a microphysiometer can be used to detect interaction of a volatile compound, present as a BP complex, with the receptor without labeling either of the compound, the carrier molecule or the receptor. As used herein a microphysiometer (e.g., Cytosensor) is an analytical instrument that measures the rate at which a cell acidifies its environment using a light-addressable potentiometric sensor (LAPS). Changes in this acidification rate can be used as an indicator of interaction between volatile compounds with a receptor. Alternatively, the Biacore system may be used. Biacore® is a Surface Plasmon Resonance (SPR)-based biosensor system dedicated to the qualitative or quantitative determination of substances in samples.

In yet another embodiment, a membrane based assay may be used to test if a volatile compound, present in a BP complex, may bind a volatile-compound-binding receptor. Cell-free assays involve preparing a reaction mixture of the studied receptor and the volatile compound as a BP complex under conditions that allow the two components to interact and bind, thus forming a further complex that can be removed and/or detected. Interactions between two molecules can also be detected, e.g., using fluorescence energy transfer (FRET). A fluorophore label is elected such that a first ‘donor’ molecule's emitted fluorescent energy will be absorbed by a fluorescent label on a second, ‘acceptor’ molecule, which in turn is able to fluoresce due to the absorbed energy. Alternatively, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between labels is related to the distance separating the molecules, the spatial relationship between molecules is related to the distance separating the molecules, the spatial relationship between the molecules can be assessed. In a situation in which binding occurs between the molecules, the fluorescent emission of the ‘acceptor’ molecule label in the assay should be maximal. A FRET binding event can be conveniently measured through standard fluorometric detection means well known in the art (e.g. using a fluorimeter). Such binding can also be detected by the Biacore system, but also by fluorescence polarization.

In the examples of the present application the functional response is measured using a fluorescent method. However, in the methods of the present invention a luminescent, radioisotope or fluorescent method may also be used.

A preferred method of the present invention may be divided into two steps: 1) Solubilization of the volatile compound by adsorption of this compound onto either a solution of proteins (secretory mucus such as olfactory mucus) or specific carrier proteins (volatile-compound Binding Protein such as serum albumin and/or Lipocalin), 2) application of the so-made complex onto cells expressing the receptor recognizing said volatile compound, or a candidate receptor which may recognize said compound. Transduced signals are then followed using either conventional fluorescence assays measuring calcium flux with calcium-sensitive dyes including Fura2, Fluo4 (Molecular Probes) and Calcium3 (Applied Biosystem), or luminescence-based assay measuring either calcium flux including aequorine-based assay (Euroscreen) and reporter-based assay such as CRE-Luciferase assay (Promega), or assessing intracellular pool of cAMP (TROPIX). Single cell signaling can be measured using single cell calcium imaging; general signals can be measures using for instance a microplate reader.

When using binding assays, the binding may be measured using a radioisotope, fluorescent (FRET, polarization) or luminescent method (BRET).

As mentioned in previous paragraphs, the cells used in the method according to the present invention may be heterologous cells; or the cell membranes used in the method according to the present invention may be prepared from heterologous cells expressing said receptor. With the term ‘heretologous cell’ is meant any eukaryotic (e.g. yeast cells, mammalian cells, avian cells, amphibian cells, plant cells, fish cells and insect cells) or prokaryotic cell (e.g. bacterial cells such as E. coli).

Alternatively, the cells used in the method of the present invention may be cells isolated from tissues chosen from the group consisting of, but not limited to, the olfactory epithelium, germ cells, testis, spleen, insulin-secreting β-cells, heart, brain, trachea, intervertebral intercosa, hit joint cartilages, liver, stomach, intestinal surface, thymus, dorsal muscles and coronaries; or when cell membranes are used said cell membranes may be prepared from said tissues. It has been previously shown that OR expression is not restricted to olfactory epithelium but has also been observed in other tissues like germ cells, testis, insulin-secreting β-cells, spleen, specific brain areas and heart. In said tissues, the function of these OR receptors still needs to be deciphered. Consequently, said cells may be used in a method of the present invention in order to identify which ligand binds to said receptors resulting in the de-orphanisation of said receptors. Besides, specific binding sites for porcupine OBP has been found in olfactory epithelium but also in many other locations such as tracheal, intervertebral, intercostals, hit joint cartilages, liver, stomach, intestinal surface, thymus, dorsal muscles, heart and coronaries. Also for these latter tissues, no clear function of OBP is known. As found in the blood, SA is consequently present in most tissues of the body. This highlights the urge to identify role(s) of specific OR, and role(s) of specific OBP and SA, more generally BP, in certain tissues.

More particular the cells used in the method of the present invention may be neurons isolated from one of said tissues; or, when cell membranes used wherein said cell membranes are prepared from said isolated neurons. Methods to purify neurons from tissues are well known in the art. Such cells can be purified either by differential centrifugations or by affinity (eg. Immunoprecipitation).

The method of the present invention may be a screening method. In a preferred embodiment, the method of the present invention may be a high throughput method. In said method volatile compounds of different chemical families may be tested. Furthermore, chemically analogous compounds may be tested in a parallel set up. The present invention demonstrates that a panel of volatile compounds may easily be tested to determine their specificity towards a receptor.

The present invention further relates to a kit comprising a cell expressing a membrane-integrated receptor recognizing a volatile compound, or a membrane fraction thereof, and, a volatile-compound-Binding Protein (BP), a complex thereof, or, a composition thereof. According to the present invention, said BP may be chosen from the group consisting of serum albumin or Lipocalin (Lipocalin-like-protein or serum-albumin-like protein). Said Lipocalin may be chosen from the group consisting of odorant binding protein (OBP), pheromone binding protein (PBP), Retinol binding protein (RBP), major urinary protein (MUP), aphrodisin, and von Ebner gland protein. Said receptor may be also a candidate receptor for recognizing volatile compound.

Alternatively, the kit according to the present invention may also be defined as comprising nucleic acid sequences coding for a membrane-integrated receptor recognizing a volatile compound, or a candidate for recognizing such as ligand; and, a nucleic acid encoding a volatile-compound-Binding Protein (BP). When nucleic acids are included in the kit, a skilled person may easily prepare recombinant proteins using a wide variety of known heterologous systems.

Furthermore, said kit may also comprise a cell expressing a membrane-integrated receptor recognizing a volatile ligand, or a membrane fraction thereof, or a receptor candidate for recognizing such as ligand; and, a nucleic acid encoding a volatile-compound-Binding Protein (BP).

Each of the kits according to the present invention may further comprise instructions for using said kit for identifying or to confirm binding and function of a volatile compound onto a membrane-integrated receptor. As used herein, the terms “instructions for using said kit” for the study of said receptors, include instructions for using the reagents contained in the kit for the study of variant and wild type receptors recognizing volatile compound, or receptor candidates for said volatile compound. The nucleic acids present in the kits of the present invention may be present in a vector or expression vector. The term ‘vector’ refers to nucleic acid molecules that transfer DNA segments from one cell to the other. The term ‘expression vector’ refers to a recombinant DNA molecule containing a desired coding sequence and appropriate nucleic acid sequences necessary for the expression of the operable linked coding sequence in a particular host organism. Nucleic acid sequences necessary for expression in prokaryotes usually include promoter, an operator (optional), and a ribosome binding site, often along with other sequences. Eukaryotic cells are known to utilize promoters, enhancers, and termination and polyadenylation signals. Said vector may be used to transfect cells. Transfection may be accomplished by a variety of means known to the art including calcium phosphate-DNA co-precipitation, DEAE-dextran-mediated transfection, electroporation, microinjection, liposome fusion, lipofection, protoplast fusion, retroviral infection and biolistics.

The present invention also elaborates on the use of the kit according to the present invention to identify and/or to confirm the binding and/or function of a volatile compound onto a membrane-integrated receptor. The terms such as ‘cell’, ‘membrane-integrated receptor’, ‘volatile compound’, ‘SA’, ‘BP’, ‘fraction’, ‘complex’ and ‘composition’ should be interpreted as explained for the method of the present invention.

In the methods, kits, or uses according to the present invention, the volatile-compound binding protein or the Lipocalin may be chosen from the group consisting of odorant binding protein (OBP), pheromone binding protein (PBP), retinol binding protein (RBP), major urinary protein (MUP), aphrodisin and von Ebner gland protein.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can be used in the practice or testing of the present invention. All publications and other references mentioned herein are incorporated by reference in their entirety. In case of conflict, the present specification, including definitions, will control. The materials, methods, and examples are illustrative only and not intend to be limiting. Other features and advantages of the invention will be apparent from the following drawings, detailed description, and from the claims.

### BRIEF DESCRIPTION OF THE FIGURES

FIG. 1: Signal transduction cascade triggered by OR activation by an odorant molecule. See text for details.

FIG. 2: Bovine olfactory mucus enhances hORL-424 activation by citronellal. (A and B) Calcium measurement was performed after activation of hORL-424 with citronellal in the presence of different amount of bovine olfactory mucus. 0 to 10 μl of mucus (0 to 10 μg of total protein) have been added to assay buffer. Calcium flux has been followed on FDSS (Hamamatsu Photonics K.K. plate reader, Japan) using Fluo4 as fluorescent dye. Histograms represent fluorescent signal observed in the presence or in the absence of 10 μl of bovine olfactory mucus. (C) Concentration response curve of HEK 293T overexpressing hORL-424 treated with citronellal. Gray boxes represent values obtained in the presence of bovine olfactory mucus. Calcium flux measurements have been performed on FDSS (Hamamatsu Photonics K.K. plate reader, Japan). (D and E) Traces obtained during single cell calcium imaging after addition of 500 μM octanal on HEK 293T overexpressing mOR-I7 in the absence (D) or in the presence of 10 μl of bovine olfactory mucus (E). Fura2 was used as fluorescent dye. Results are expressed as F340/F380 ratiometric measurement.

FIG. 3: Development of a novel olfactory functional assay. (A) Scheme of the novel assay according to the present invention, CCBSA. See details in the text. (B) Application of CCBSA on a microplate format fluorescent cell-based assay. Increasing volume of octanal were incubated in CCBSA, and applied on HEK 293T cell line overexpressing or not mOR-I7. Calcium flux was followed with fluo4 as calcium tracer. (C) Application of CCBSA on single cell calcium imaging assay. Octanal was incubated in CCBSA and applied on HEK 293T overexpressing mOR-I7. Upper panel depicts kinetics of calcium flux followed with fluo4 as calcium tracer. Octanal-incubated bovine olfactory mucus was applied at 50 s and ATP as positive control was applied at 310 s. Forty X objective was used for this experiment. Middle panel show kinetics of 20 individual cells taken off the field represented in the upper panel. Finally, a representation of the average of the 20 traces is given in the lower panel.

FIG. 4: Fractionation of bovine olfactory mucus. (A) Co-elution of OBP and Bovine Serum Albumin (BSA) during bovine olfactory mucus fractionation on DEAE (peaks 4 and 5). Protein elution has been followed at 280 nm. Enclosed is a SDS-PAGE showing profile of the 6 different peaks detected during the fractionation. OBP and BSA have been identified by mass spectrometry. (B) Purification of bovine OBP (bOBP). Peaks 4 and 5 have been pooled and fractionated on C18. Protein elution has been followed at 214 nm. bOBP and BSA were eluted separately as depicted by the enclosed SDS-PAGE. Also, bOBP was eluted into two peaks. OBP and BSA have been identified by mass spectrometry.

FIG. 5: Two fractions of bovine olfactory mucus that mainly contain OBP and BSA enhance mOR-I7 activation in the assay according to the present invention. Screening of 30 odorant molecules on HEK 293T wt or overexpressing mOR-I7 with the assay according to the present invention, CCBSA. Fluo4 was used as fluorescent dye during this screening. (A) List of screened molecules. (B and C) depicts kinetics observed at 520 nm for HEK 293T mOR-I7 and HEK 293T Wt during the assay performed on FDSS (Hamamatsu Photonics K.K., Japan), respectively. (D) Histograms representing cellular responses to the different odorants listed in (A). Results are expressed as ratio of mOR-I7 on Wt after standardization to response triggered by injection of 1 mM ATP. (E) Schemes of different hits obtained during the screening. (a) 6-cis-nonenal, (b) citronellal, (c) 4-(2-methoxyethyl)-phenol, (d) Vanillyl acetone, (e) citronellol, (f) 2,3 heptanedione, (g) 1-octanal, (h) 1-octanol, (i) 1-heptanol, (j) olfactophore 1 and (k) olfactophore 2. (F) Depicts kinetics observed at 520 nm for HEK 293T mOR-I7 during a classical assay performed on FDSS (Hamamatsu Photonics K.K., Japan). A classical assay means odorant molecules are directly loaded into the assay buffer without being subjected to CCBSA assay.

FIG. 6: BSA is an odorant carrier protein. (A, B and C) mOR-I7 activation by bovine olfactory mucus peak 4 and 5 incubated with different amount of octanal in the assay as described by the present invention. (D, E and F) mOR-I7 activation by a solution of BSA incubated with different amount of octanal in our novel assay. Panels A and D show kinetics of HEK 293T overexpressing mOR-I7 while panels B and E show kinetics of HEK 293T Wt under the same conditions. Kinetics is expressed as standardized fluorescence intensity. (G) BSA per se does not trigger any calcium flux at the concentration used in the assay as described by the present invention. CCBSA was performed with increased amount of BSA in the absence of any odorant. Results are expressed as fluorescent units. The arrow pinpoint the concentration used in the assay described in the present invention. (H) Kinetics of cAMP synthesis after incubation of mOR-I7 with 5 different ligands, among which the 3 known ligands of mOR-I7, octanal, heptanal and citronellal. cAMP concentration has been determined by immunoassay (TROPIX, Applied Biosystem). Results are expressed as nM per well. (I) Histogram representing concentration of cAMP synthesized after 20 minutes incubation with 5 different ligands. Results are expressed as nM per well.

FIG. 7: Screening of 30 odorant molecules on HEK 293T overexpressing mOR-I7 with the cell based olfactory functional assay as described in the present invention using BSA as odorant carrier protein. (A) List of the 30 screened odorant molecules. (B) Representative plate kinetics as shown on plate reader window during screening (FDSS, Hamamatsu Photonics K.K., Japan). (C) mOR-I7 activation has been followed by calcium flux using fluo 4 as fluorescent dye on FDSS (Hamamatsu Photonics K.K., Japan). Results of two independent screenings are represented. They are expressed as percent of cellular response after injection of 100 μM ATP. Bars noted with a star represent consistent responses obtained from two independent screenings. See meaning of grey bars in the text. (D) mOR-I7 activation has been followed by determination of intracellular cAMP concentration using an immunodetection assay (TROPIX, Applied Biosystem). Results of two independent screenings are represented. They are expressed as percent of cellular response after incubation with 100 μM forskolin. Bars noted with a black circle represent consistent responses obtained from two independent screenings. See meaning of grey bars in the text. (E) Schemes of different hits obtained during the different screenings. (a) citronellal, (b) 1-octanal, (c) 1-octanol, (d) 2,3 heptanedione, (e) 1-heptanal, (f) 1-heptanol, (g) olfactophore.

### MODES FOR CARRYING OUT THE INVENTION

### Example 1

### Materials and Reagents

Reading of plates in the fluorescence-based assays described in the present invention has been performed on the FDSS system (Hammatsu, Japan). Reading of plates in the luminescence-based assays described in the present invention (cAMP level measurement) has been performed on Fluostar (BMG, Germany). All reagents including BSA fatty-acid free and odorants have been purchased from Sigma unless otherwise specified in the text.

### Example 2

### Cell Culture

HEK 293T cells were routinely grown in DMEM complemented with FBS at 37° C. under 5% CO2 and 90% humidity. Two days before functional assay, cells were plated onto 96-well plates (view-plate, Packard) to confluence, and kept at 37° C. in culture medium.

### Example 3

### Bovine Olfactory Mucus Sampling

Sample of bovine olfactory mucus was obtained from healthy dead cow (Viangro slaughterhouse, Brussels, Belgium). Bovine olfactory epithelium lays in the uppermost part of the nasal cavity. Cow muzzle was cut off the head and then longitudinally divided to evidence olfactory cleft. Olfactory mucus was then sampled. Pool of forty different mucus samples was used to perform experiments described in the present invention.

### Example 4

### OBP Purification

After a centrifugation at 4° C., pooled olfactory mucus was desalted on G25 matrix. Elution buffer was made of 50 mM Tris-Cl pH 8.0, 2 mM EDTA, 10% glycerol, 1 mM PMSF and 1 mM DTT. One mg proteins were then applied to a DEAE matrix (Amersham Bioscience). Protein elution was performed by a gradient of NaCl (10 mM to 500 mM) and detected at 280 nm. OBP and BSA-containing fractions were further fractionated on C18 matrix. Proteins were eluted by an acetonitrile gradient (0% to 90%) in the presence of 0.1% formic acid and 4 mM ammonium acetate.

### Example 5

### Fluorescence-Based Functional Assay

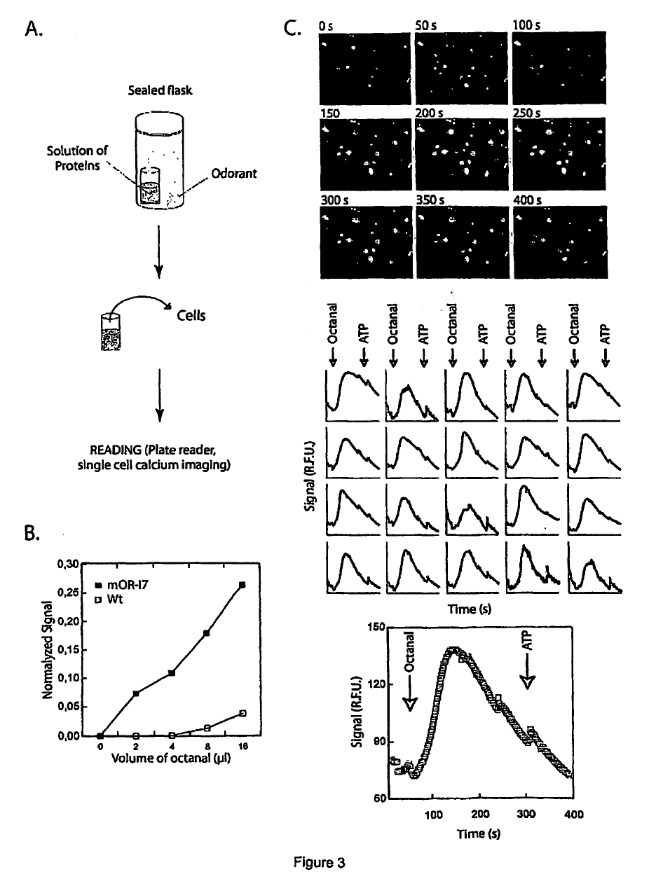

A solution of BP including SA, OBP and/or olfactory mucus (see concentration in the text) was incubated in the presence of odorant in a sealed flask for 16 hours at 4° C. (FIG. 3A). Such incubated-BP solution was then used as tested complex on olfactory receptor-expressing cells. Prior to incubation with such complex, cells were incubated for 1 hour with 4 μM Fluo4-AM (Molecular Probe) in the presence of 1 mM probenecid, and then rinsed twice with Fluo4-AM free HBSS buffer. After injection of odorant-BP complex on OR expressing cells, calcium flux was followed by measuring fluorescence at 520 nm (excitation 380 nm) on FDSS, a plate reader from Hamamatsu Photonics K.K. (Japan) at 25° C.

### Example 6

### cAMP Level Measurement in Our Novel Assay

Similarly to the first step of the fluorescence-based functional assay described above, BP solutions including SA, OBP and/or olfactory mucus (see concentration in the text) were incubated in the presence of odorant in a sealed flask for 16 hours at 4° C. (FIG. 3A). Such odorant-BP complexes were then applied to cultured-cell for 20 minutes at 37° C. cAMP level was then measured using the Tropix kit according to the manufacturer procedures (Applied Biosystem).

### Example 7

### Bovine Olfactory Mucus Improves Olfactory Receptor Activation by its Ligand

In the prior art it has been suggested that odorant molecules may bind the binding pocket of OBP. However, there is no hint in the literature if they are still accessible to olfactory receptors. Furthermore, suggestions were made in the literature that those odorant molecules may be trapped by OBP to prevent olfactory receptor activation. To address these hypothesise, the ability of bovine olfactory mucus to prevent olfactory receptor activation has been first assessed.

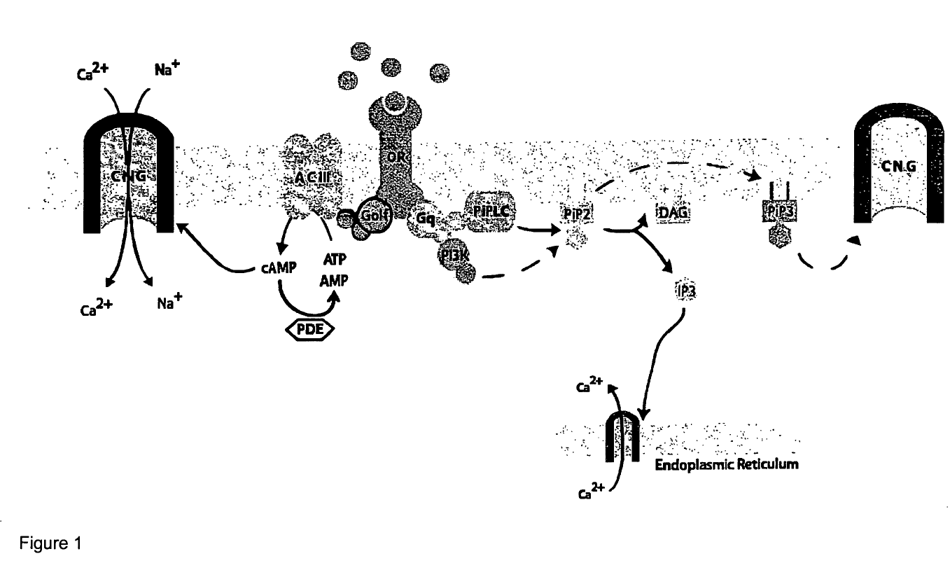

hORL-424, a human olfactory receptor, was overexpressed in HEK 293T, a cell line derived from rat olfactory epithelium (Hunter). Once differentiated, HEK 293T expressed the all panel of transduction proteins necessary for signal transduction, and thereof allow detection of OR activation through calcium flux (FIG. 1).

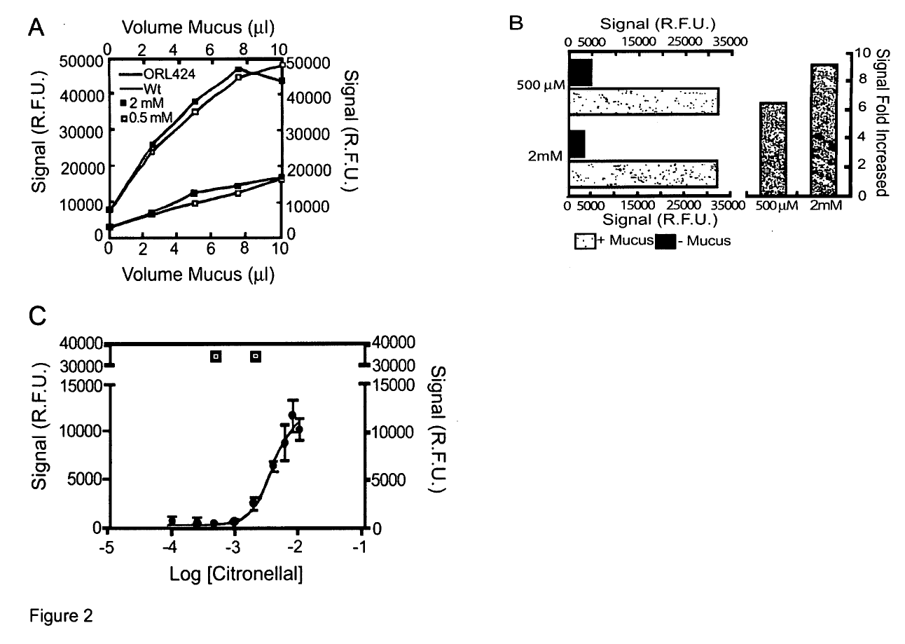

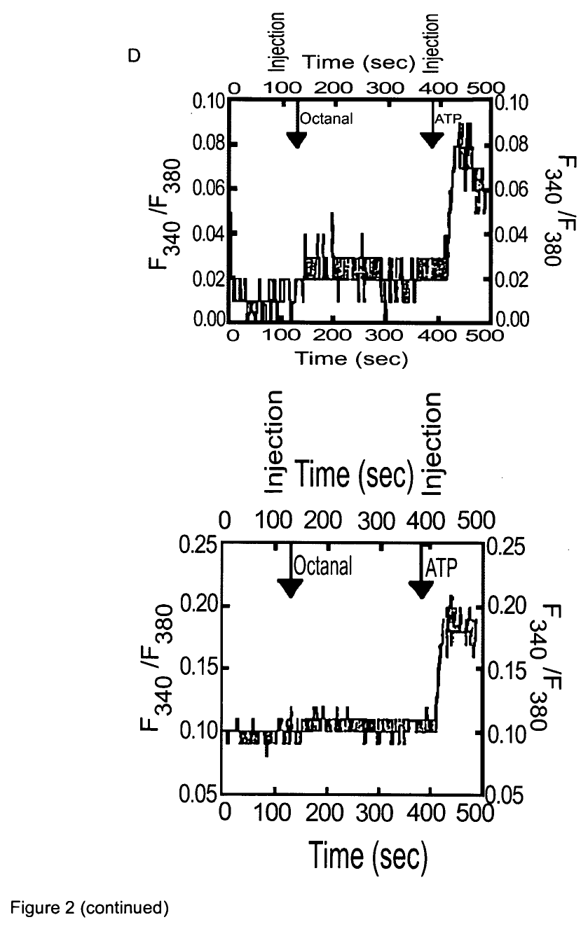

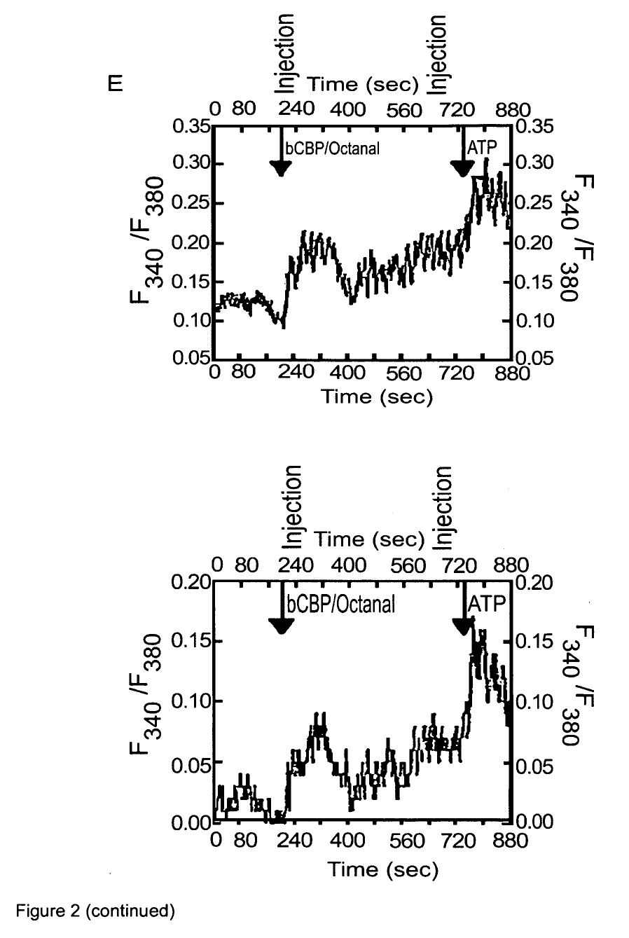

Activation of hORL-424 by citronellal in the presence of increasing amount of bovine olfactory mucus was followed by a fluorescent calcium tracer, fluo4, in a 96-well plate format. Addition of bovine olfactory mucus in the assay buffer led to a specific increase of hORL-424 activation by citronellal (FIG. 2A). Receptor activation was improved by approximately 9 folds (FIG. 2B). In addition, while up to 10 mM were necessary to reach maximum activation of hORL-424 in a conventional assay, as low as 0.5 mM was sufficient when olfactory mucus was added in the assay buffer (FIG. 2C).

This bovine olfactory mucus property was also observed on a different OR in other assay format. FIGS. 2 D and E show traces obtained from single cell calcium imaging performed on HEK 293T overexpressing mOR-I7 and activated by octanal. In this assay, Fura2 was used as calcium tracer. While octanal triggered a faint activation of mOR-I7 in the absence of mucus (FIG. 2D), a calcium flux similar to the one observed after addition of ATP, a positive control was observed in the presence of mucus (FIG. 2E).

The results depicted on FIG. 2 thus demonstrate that addition of bovine olfactory mucus in buffer of a conventional functional assay specifically improved olfactory receptor activation.

### Example 8

### Development of a Novel Olfactory Functional Assay

In an attempt to be closer to physiological process of olfaction and thereof to increase specificity and sensitivity of the cell-based assay described above, a novel olfactory functional assay was developed. Most of the time, mammalian smells volatile odorant molecules. However in the cell-based assay described above as in most cell-based assays, odorant molecules were injected into aqueous buffer. Such injection may lead to formation of micelles that can alter efficacy and specificity of ligands. Therefore a cell-based assay was developed focusing an a better solubilization of odorants. In summary, in a sealed flask, bovine olfactory mucus was incubated in the presence of an odorant (FIG. 3A). It was hereby tested if an odorant would be trapped and solubilized in that fluid. Thereafter, such solubilized odorant was injected onto cultured-cells overexpressing an olfactory receptor. Activation of this receptor could then be followed by conventional assay including fluorescent (FIGS. 3 B and C) and luminescent calcium assays.

For this novel assay, mOR-I7 activation was assessed by octanal. A constant volume of olfactory mucus was incubated in the presence of increasing amount of octanal ranging from 0 to 16 μl of a solution of 1M octanal. After 16 hours incubation at 4° C., bovine olfactory mucus was injected on HEK 293T overexpressing mOR-I7. Calcium flux was followed with Fluo4 on FDSS, a plate reader from Hamamatsu Photonics K.K. (Japan). As shown on FIG. 3B, fluorescence intensity was proportional to the volume of odorant incubated with bovine olfactory mucus. This cell response is specific to mOR-I7 activation by octanal since no cell response was observed on wt cells. Moreover, bovine olfactory mucus does not trigger any unspecific cell response per se since no calcium flux was observed after injection of buffer-Incubated bovine olfactory mucus on cells (FIG. 3B).

To further validate this assay, single cell calcium imaging assays following the same approach were performed (FIG. 3C). Bovine olfactory mucus was incubated in the presence of 16 μl of a 1M octanal solution for 16 hours at 4° C. Bovine olfactory mucus was then applied onto HEK 293T overexpressing mOR-I7. Calcium flux was followed with the Fluo4 calcium tracer and cells were observed at 40× magnification. One mM ATP was injected as positive control at the end of the experiment. Pictures of a representative field of the time course experiment is shown in the upper panel. Analysis of kinetics of fluorescence intensity of 20 cells taken off this field demonstrates that cells overexpressing mOR-I7 are activated by bovine olfactory mucus incubated with octanal. An average of these 20 kinetics is depicted on the lower panel.

This novel assay allows thus an efficient solubilization of odorant and greatly improves the efficacy of the molecule. In fact while fluorescence intensity observed after positive control injection such as ATP is usually greater than that observed after odorant injection in a conventional assay (FIGS. 2 D and E), single cell calcium imaging results (FIG. 3C) show that odorant in the newly proposed assay triggers a bigger calcium flux than ATP. Such disproportion has never been detected in a conventional assay. Also in the novel assay brings much more sensitivity compared to assays wherein than mucus is simply added in the assay buffer (compare FIGS. 2 E and D with FIG. 3C). This observation suggests that solubilization of odorants is really a critical step in olfactory functional assay.

### Example 9

### Fractions of Bovine Olfactory Mucus Containing OBP and BSA Improve Olfactory Receptor Activation

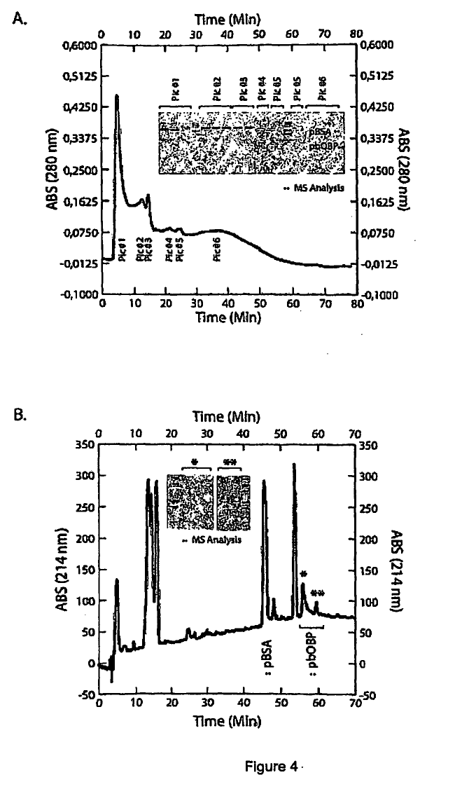

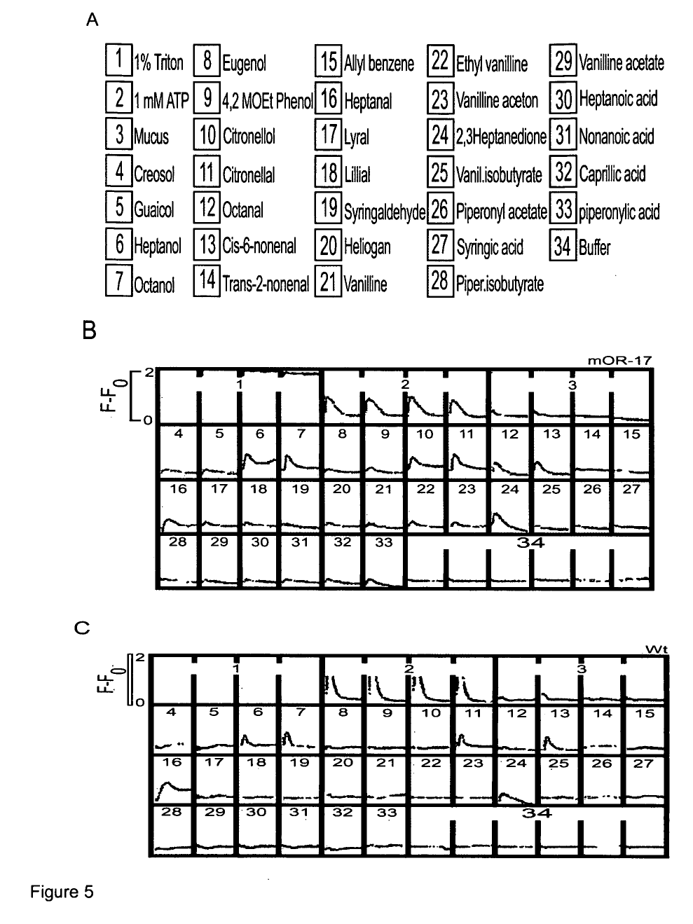

Results shown in FIG. 3 demonstrate that bovine olfactory mucus is capable of solubilizing odorant molecules. The present invention suggests that this property is due to the presence of a large amount of OBP and/or SA. To test this hypothesis, bovine olfactory mucus has been fractionated to isolate bovine OBP (bOBP). Elution from a DEAE column generated 6 major peaks. Peaks 4 and 5 contained mainly bOBP and BSA as identified by mass spectrometry (FIG. 4A). Since elution profile of those two peaks was very similar, they were pooled and named fraction 4.

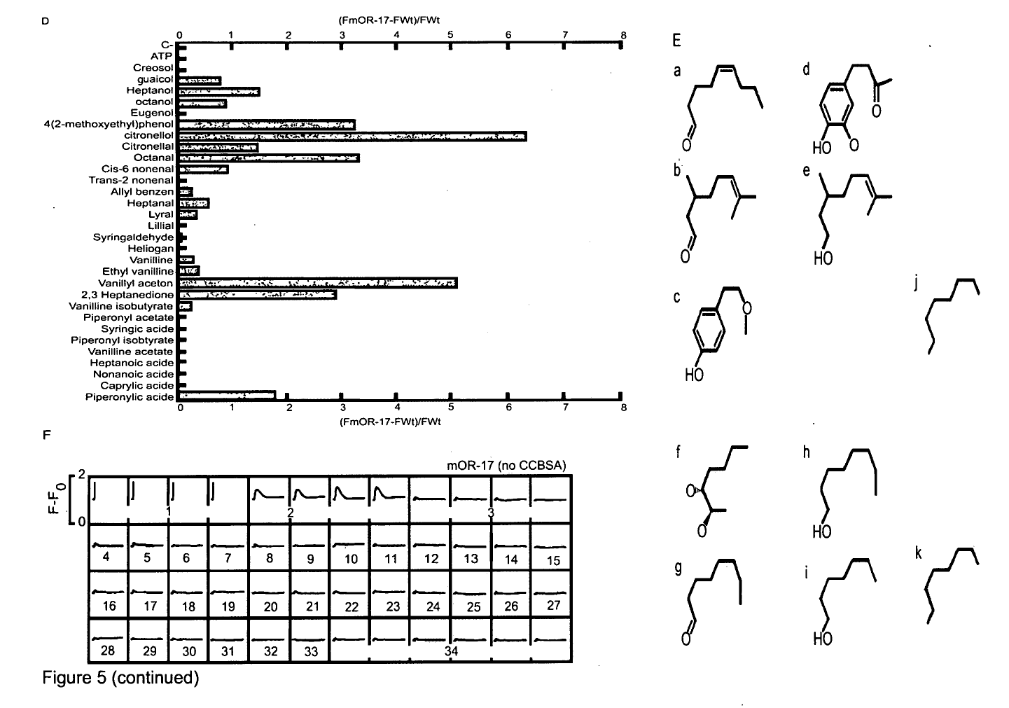

Capacity of fraction 4 to fulfil the role of odorant solubilizator was assessed in the newly proposed assay. Screening of 30 odorant molecules (FIG. 5A) was performed on HEK 293T wild type (FIG. 5C) or overexpressing mOR-I7 (FIG. 5B). Fraction 4 was incubated overnight at 4° C. In the presence of 16 μl of 1M solution of the 30 different odorants listed in FIG. 5A. For comparison, FIG. 5F shows traces usually obtained when odorant are directly loaded into the assay buffer instead of performing the CCBSA as described in the present invention. mOR-I7 activation was followed on FDSS (Hamamatsu Photonics K.K., Japan) with fluo4 as fluorescent dye. While no activation is detected under basic experimental conditions (FIG. 5F), ten odorants came up as potential ligands of mOR-I7: heptanol, octanol, 4(2-methoxyethyl)phenol, citronellol, citronellal, octanal, cis-6-nonenal, Vanillyl acetone, and 2,3 heptanedione (FIGS. 5D and E). These odorants led to at least one fold more activation of HEK 293T overexpressing mOR-I7 than the wild type counterpart. They can be classified into two groups. One group (FIG. 5 E (a) to (e)) gathers molecules that fit the olfactophore 1 depicted in FIG. 5 E (j). They are schematically made of 2 electronegative poles distanced by 6 carbons. Another group is made of molecules shown in FIG. 5 E (f) to (i). They all contain a 6-8 carbon chain ended by an electronegative group as depicted by olfactophore 2 represented in FIG. 5 E (k).

### Example 10

### BSA is a Novel Odorant Carrier Protein

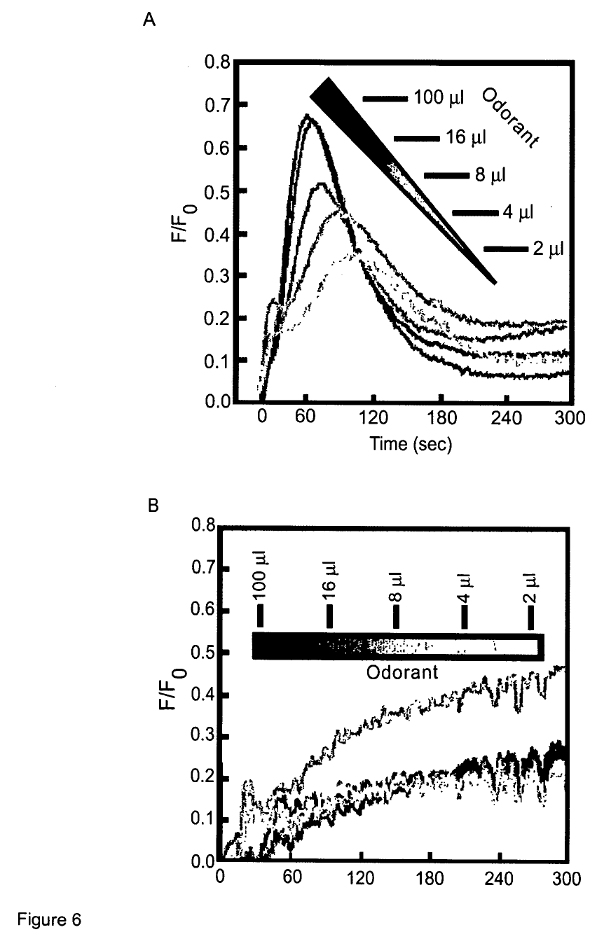

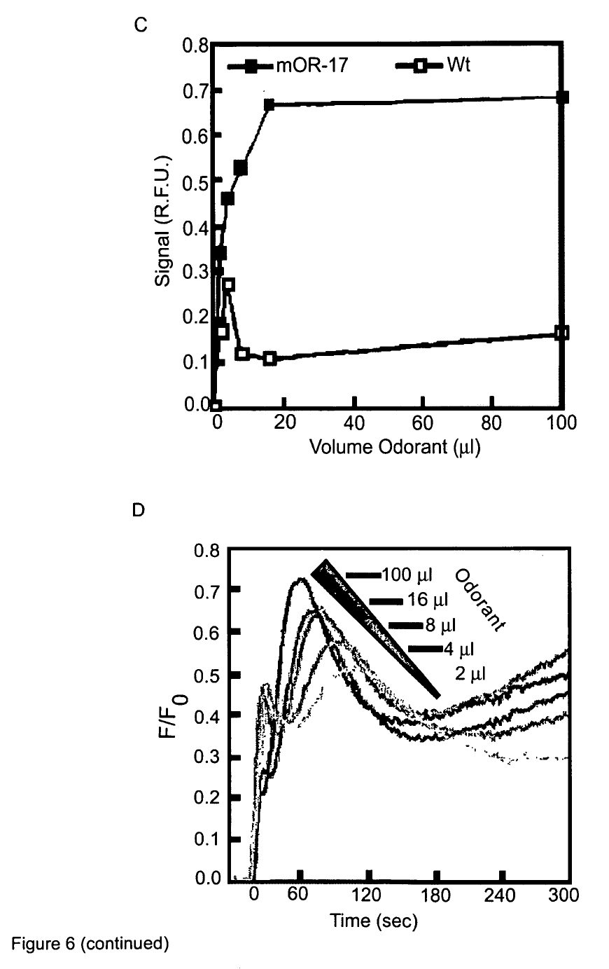

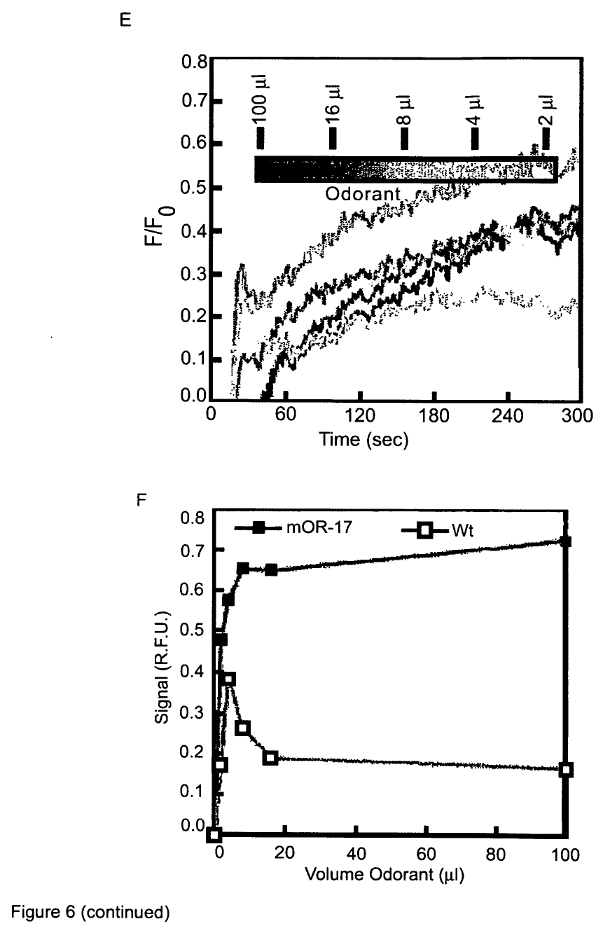

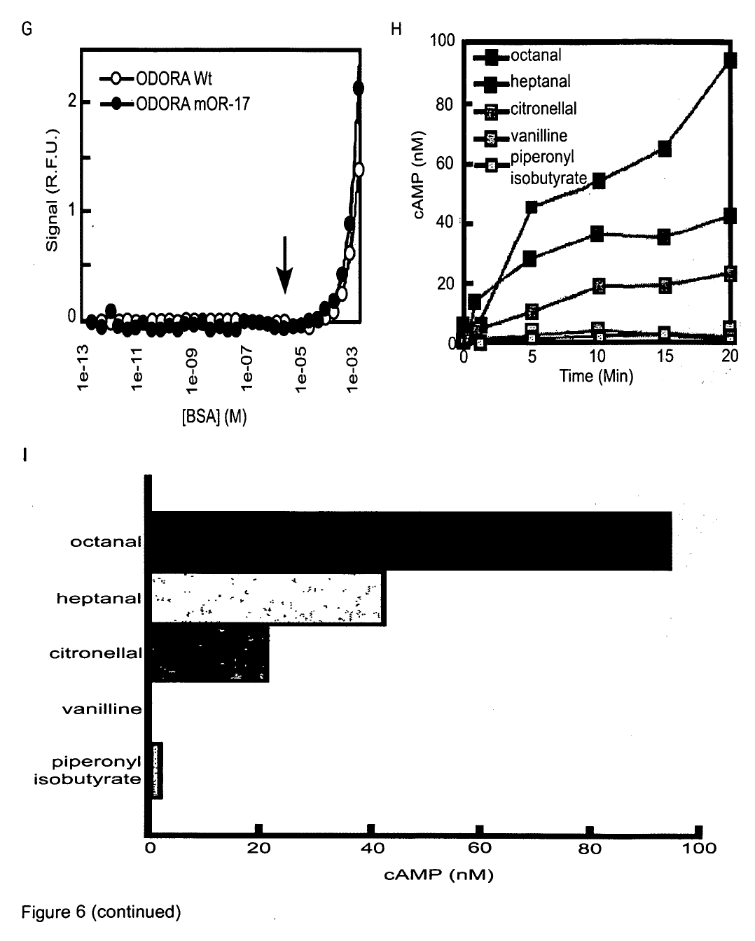

Example 9 showed that fraction 4 had properties to solubilize and enhance efficacy of odorant molecules in the novel cell based olfactory functional assay (FIG. 5). However this fraction 4 contained two major proteins, bOBP and BSA. Thus the results of said Example could in this approach not rule out any implication of BSA into fraction 4 properties. Therefore the capacity of BSA to solubilize and thereof to enhance the efficacy of odorants in the novel cell based olfactory functional assay as described by the present invention has been tested. To be as close as possible to BSA concentration observed in fraction 4. BSA concentration was set up at 5×10⁻⁶ M for the following experiments (marked by an arrow on FIG. 6 G). Fraction 4 and a solution of fatty acid-free BSA were incubated at 4° C. for 16 hours in the presence of increasing volume of 1M octanal. So-incubated solutions were then applied onto HEK 293T wt or overexpressing mOR-I7. Calcium-flux was followed on a plate reader (Hamamatsu Photonics K.K., Japan) with fluo4 as fluorescent dye (FIG. 6 A to F). Kinetics over 300 seconds (FIGS. 6 A, B, D and E) showed that while calcium flux was detected in HEK 293T overexpressing mOR-I7, no fluorescence appeared in the wt counterpart. Also intensity of detected fluorescence was proportional to the amount of odorant engaged in the assay. Inversely, time required to reach maximum fluorescence decreased when odorant volume increased (FIGS. 6 A and D). Such phenomenon is usually observed in such fluorescence assay when increasing ligand concentrations are injected.

Fluorescence signals detected during this kinetics are not due to BSA per se. In fact, in the one hand no calcium flux was detected in HEK 293T wt. On the other hand solutions ranging from 10⁻¹³ to 10⁻⁴ M of odorant-free BSA did not trigger any fluorescent signal (FIG. 6G). The concentration of BSA used in these assays was 5×10⁻⁶ M as described above.

Concentration-response curves obtained with either fraction 4 or BSA solution were very similar (FIGS. 6 C and F). As low as 20 μl of 1M odorant solution are sufficient to trigger maximum response of mOR-I7 in the new system with either of those solubilizing solutions.

As depicted in FIG. 1, binding of odorant molecule to olfactory receptor triggers a cascade of molecular events. In the most commonly accepted pathway, the first second messenger is cAMP synthesized by adenylate cyclase type III. Therefore, a mean to test olfactory receptor activation is to assess variation of cellular cAMP pool after olfactory receptor activation. It is noteworthy that such assay could be performed in parallel to calcium flux assay described above to validate hits obtained during a screening.

mOR-I7 is known to be activated specifically by 3 odorants: octanal, heptanal and citronellal (references). Specificity of the novel cell based olfactory functional assay according to the present invention was tested following mOR-I7 activation after injection of these three odorants and two non related odorant, vanilline and piperonyl isobutyrate. Olfactory receptor activation was detected by cellular cAMP pool immunoassay. A solution of BSA was incubated overnight at 4° C. In the presence of 16 μl of 1 M odorant solution, and then applied to HEK 293T overexpressing mOR-I7. Kinetics of mOR-I7 activation by each of these 5 odorants is shown in FIG. 6 H. Although five minutes incubation with solubilized odorant are sufficient to discriminate agonists from non agonists, cAMP accumulation last to 20 minutes. Histogram showed in FIG. 61 represents cellular cAMP pool after 20 minutes incubation with solubilized odorant. As described in the literature, our novel cell based olfactory functional assay pinpointed octanal, heptanal and citronellal as mOR-I7 ligands. Vanilline and piperonyl isobutyrate did not lead to cAMP synthesis and thereof are not ligands of mOR-I7 as expected based on their unrelated structure to the three ligands of mOR-I7 and on the literature. Taken together, these results demonstrated that a solution of BSA can be used to solubilized odorant molecules to trigger specific response of olfactory receptors.

### Example 11

### The Novel Olfactory Functional Assay Using BSA as Odorant Carrier Protein Allows Sensitive Screening of Odorant Molecules

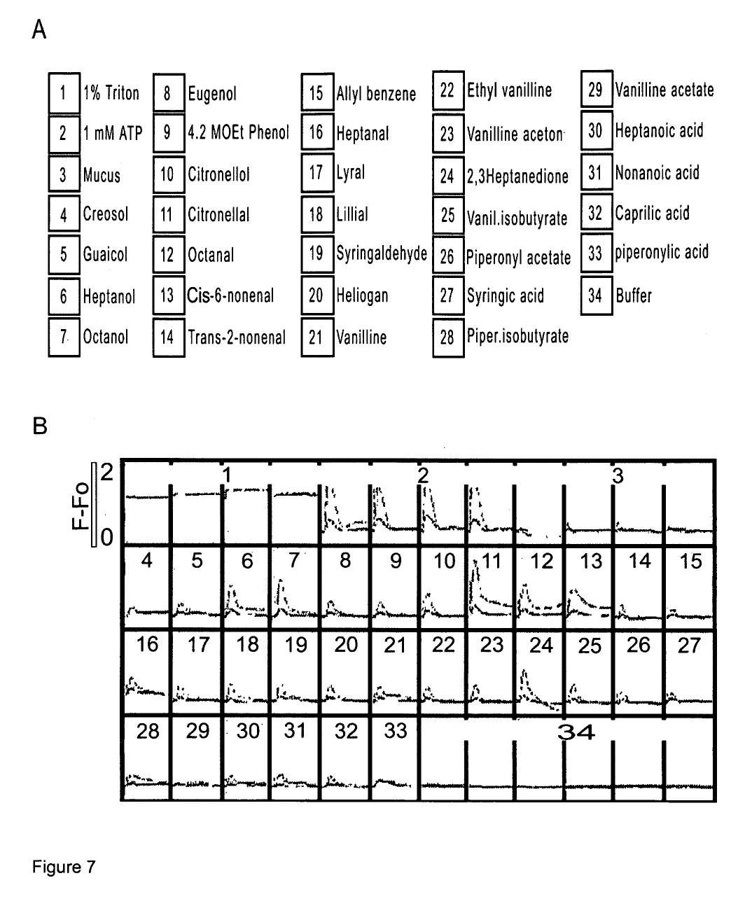

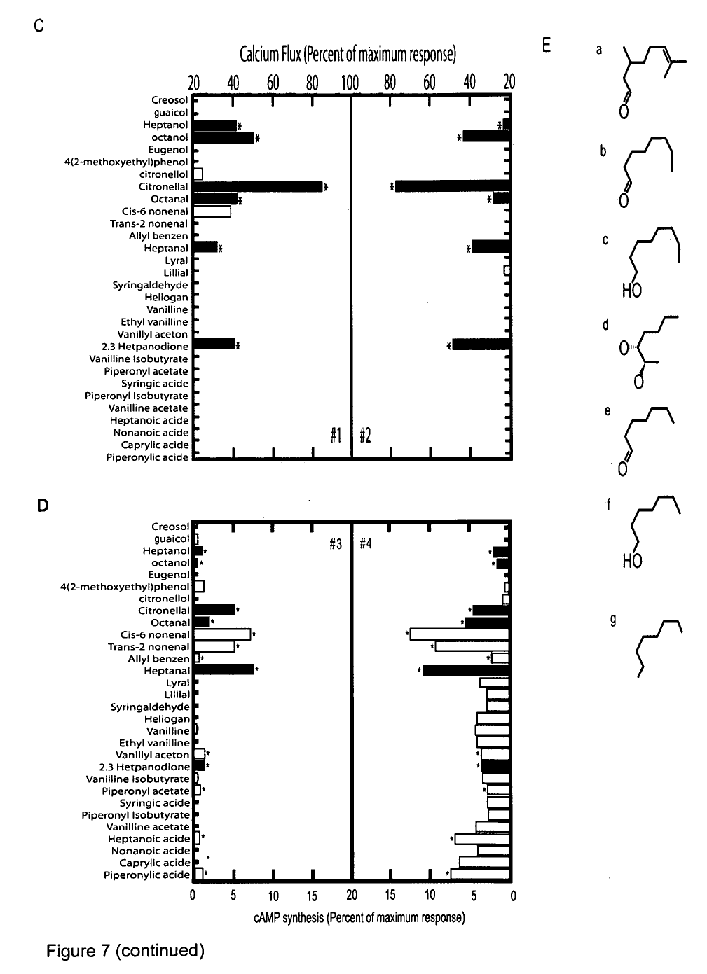

To further validate BSA as an odorant carrier protein, 4 independent screenings of 30 odorant molecules on mOR-I7 have been performed. Activation of mOR-I7 overexpressed in HEK 293T has been followed either by calcium flux assay (FIGS. 7 B and C), or by cAMP synthesis immunodetection (FIG. 7D). As described above, BSA solutions were incubated overnight at 4° C. with 30 odorant molecules listed in FIG. 7A. Solutions of BSA were then applied to cultured HEK 293T overexpressing mOR-I7. Receptor activation was either followed with a plate reader for calcium flux assay using Fluo4 as fluorescent dye (FDSS, Hamamatsu Photonics K.K., Japan), or by immunodetection assay (TROPIX, Applied Biosystem) for cAMP synthesis assay. FIG. 7B shows a typical screen observed during a screening of those 30 molecules with a calcium flux assay on FDSS (Hamamatsu Photonics K.K., Japan). Results of two independent screenings using calcium flux assay are shown on FIG. 7C. Fluorescence intensities have been standardized to signal obtained after injection of 100 μM ATP, a positive control. Eight and 7 odorants have been pinpointed as ligands of mOR-I7. Six of these odorants seem consistent 1-heptanol, 1-octanol, citronellol, 1-octanal, 1-heptanal, 2,3 heptanedione. They are star-marked in histogram represented in FIG. 7 C.

As discussed above (FIG. 1), activation of olfactory receptor can also be followed through cAMP synthesis. Such assay has been performed for screening the 30 odorant molecules on HEK 293T overexpressing mOR-I7. Results of two independent screenings are shown on FIG. 7D. Results are expressed as percent of response observed after incubation with 100 μM forskolin, an activator of adenylate cyclases. Many odorants triggered synthesis of cAMP. However, only 13 of them have been found consistent between the two screenings. They are marked by a black circle on FIG. 7D. Among these 13 odorants, 6 are found to be potential ligands of mOR-I7 through the 4 screenings done with either of the performed assay. Bar represented cell response to these 6 ligands are grey-coloured (FIGS. 7 C and D). Scheme of the odorant molecules are depicted in FIG. 7E. Comparison of their structure highlights a common backbone: an aliphatic chain ended by an electronegative group including aldehyde and alcohol.

It is noteworthy that those 6 odorants were found being ligands of mOR-I7 during screenings performed with fraction 4 as odorant carrier (FIG. 5). This observation thereof confirms that a solution of BSA play the role of odorant carrier in our novel olfactory functional assay.

### Example 12

### Assay to Determine if a Protein May be Considered as Volatile-Compound Binding Protein

Many ways exists to determine if a protein binds a volatile-compound:
1) The following proposed methods may be used to determine whether the tested protein works as a volatile-compound binding protein (BP).
2) Secondly, fluorescence polarization may be used to detect whether a compound bind a candidate protein. In such case, fluorescence polarization would increase. Incubation of the volatile compound may be performed in the same conditions described for the first step of the method of the present invention that is: a solution of tested protein is incubated in the presence of volatile compound in a sealed flask at 4° C. for 16 hours. The same experiment may be performed replacing tested protein solution with buffer alone. Fluorescence polarization may be performed and polarization coefficient of the two samples compared. Alternatively, a similar experiment can be done but dissolving directly volatile compound into tested-protein solution.
3) Third, an assay developed by Pernollet and collaborators can be used: Volatile Odorant Binding Assay (Eur. J. Biochem 267, 3079-3089, 2000). Basically, a fluorophore binds candidate protein. This fluorophore can be but is not limited to DAUDA, DACA, ASA, 1-AMA, 1,8 ANS and NPN. Emission spectrum of said free-fluorophore differs from said protein-bound-fluorophore. According to emission wavelengths one can determine whether the fluorophore is free or protein-bound. Thus, if the protein-bound-fluorophore can be displaced through the incubation of said complex with a volatile compound, the tested protein may be considered as a volatile-compound binding protein. In detail the assay may be performed as follows: as a first step, fluorophore binding experiment is performed with 2 μM tested-protein solution in 50 mM potassium phosphate buffer, pH 7.5. Fluorescent probe are dissolved in 10% (v/v) Methanol as 1 mM stock solution. One to 10 μM probe are added to the tested-protein solution and further incubated for 5 minutes at room temperature. In a second step, so-made fluorophore-protein complex is incubated in the presence of volatile compound in a sealed flask at 4° C. for 16 hours. This last step aimed to displace fluorophore with volatile compound. This displacement is detected by a shift in the fluorescence emission spectrum of the fluorophore.
4) Incubation of the volatile compound should be done in the same conditions described for the first step of our assay that is: a solution of tested protein is incubated in the presence of volatile compound in a sealed flask at 4° C. for 16 hours. Protein solution is then extracted with chloroform and analyzed by gas chromatography. A control of this experiment can be the buffer used to solubilize tested protein but alone.
5) Biacore technology can also be a mean to detect interaction between tested protein and volatile compound.

### Example 13

### Method to Identify Proteins Belonging to the Lipocalin Family

Many Lipocalin proteins have been identified such as odorant binding protein (OBP), pheromone binding protein (FBP), retinol binding protein (RBP), major urinary protein (MUP), aphrodisin, and von Ebner gland protein. Based on functional activity or structural similarity, a skilled person may easily determine if a protein belongs to the Lipocalin family or not.

For instance sequence similarity searches may be performed using the BLAST software package. Identity and similarity percentages may be calculated using BLOSUM62 as a scoring matrix. As known in the art, “similarity” between two polypeptides is determined by comparing the amino acid sequence and its conserved amino acid substitutes of one polypeptide to the sequence of a second polypeptide. Moreover, also known in the art is “identity” which means the degree of sequence relatedness between two polypeptide or two polynucleotide sequences as determined by the identity of the match between two strings of such sequences. Both identity and similarity can be readily calculated. While there exist a number of methods to measure identity and similarity between two polynucleotide or polypeptide sequences, the terms “identity” and “similarity” are well known to skilled artisans (Carillo and Lipton, 1988). Methods commonly employed to determine identity or similarity between two sequences include, but are not limited to, those disclosed in “Guide to Huge Computers (Bishop, 1994) and Carillo and Lipton (1988). Preferred methods to determine identity are designed to give the largest match between the two sequences tested. Methods to determine identity and similarity are codified in computer programs. Preferred computer program methods to determine identity and similarity between two sequences include, but are not limited to, GCG program package (Devereux et al., 1984), BLASTP, BLASTN and FASTA (Altschul et al, 1990). Proteins having 55%, 60%, 65%, preferably 70%, 75%, 80%, 85%, 90%, or 95%, or more preferably 99% similarity to known Lipocalins may be considered as belonging to the same protein or gene family.

### Example 14

### Method to Identify Proteins Belonging to the Serum Albumin Family

Many serum albumin proteins have been identified (see above). Based on functional activity or structural similarity, a skilled person may easily determine if a protein belong to the serum albumin family or not. The identification of the similarity and/or the identity between a polynucleotide or polypeptide sequence and a known SA sequences may be determined as mentioned for Lipocalin sequences in Example 13.

Its is to be understood that while the invention has been described in conjunction with the detailed description thereof, the foregoing description is intended to illustrate and not limit the scope of the invention, which is defined by the scope of the appended claims. For instance, in the above described examples heterologous HEK 293T cells overexpressing hORL-424 or mOR-I7 are used. Said receptors are known to be activated by citronellal and octanal respectively. As for said receptors the ligand was known, said receptors have been selected as reference volatile-compound-binding receptor to develop the presently described methods. However, that BP and/or SA may be used as carrier molecule in in vitro assays may also be applied for other OR, the present invention further suggests that these carrier molecules may be applied in vitro methods for all possible receptors recognizing volatile compounds. Furthermore, HEK 293T in which said receptors are expressed is only an example of possible cells which may be used. In addition, a functional assay was used for this purpose, however the effect seen may also be applied for in vitro assays using cell membranes comprising said receptors. In said experiments Ca2+ and cAMP were measured, but as a membrane integrated receptor may stimulate different signalling molecules, the measure of other signalling molecules than Ca2+ and cAMP is possible.

All of the references cited in the description are incorporated by reference. Other aspects, advantages, and modifications are within the scope of the following claims.

TABLE 1

Sequence 1: wild type murine OBP nucleic acid sequence
ATGGTGAAGTTCCTGCTAATTGCGATTGCATTAGGTGTATCCTGTGCACATCATGAATCTCTTGA
TATCAGTCCCTCAGAGGTTAATGGGGACTGGCACACCCTTTACATAGCTGCAGACAAGGTGGAGA
AAGTAAAGATGAATGGAGACCTGAGAGCGTACTTTGAGCATATGGAGTGCAATGACGACTGTGGG
ACACTCAAAGTCAAATTCCATGTCCAGATGAATGGCAAGTGTCAGACACACACTGTTGTGGGAGA
AAAACAAGAAGATGGGCGGTACACTACTGACTGTGAGTATAAATTCGAAGTTGTAATGAAGGAAG
ATGGCGCCCTTTTCTTTCACAACGTTAATGTGGATGAGAGCGGACAGGAGACAAATGTGATTTTA
GTTGCTGGAAAAGGAGAGACCCTGAGCAAAGCACAGAAGCAGGAGCTTGGGAAGCTGGTCAAGGA
ATACAATATTCCAAAGGAGAATATCCAGCACTTGGCACCCACAGGTTTTAAAACTGTTGTACTCA
TCTGGGCACTGCAGACAGATGGGCCATGGAAAACTATAGCTATCGCTGCTGATAATGTAGACAAA
ATAGAGATTAGTGGAGAGGACAAAATAGAGATTAGTGGAGAGCTGAGGCTCTATTTTCATCAAAT
TACTTGTGAAAAGGAATGCAAGAAAATGAATGTCACATTTTATGTCAATGAAAATGGACAATGTT
CATTGACAACAATCACTGGGTATTTGCAAGATGATGGCAACACCTACAGATCCCAATTTCAAGGG
GATAATCATTATGCAACTGTGAGGACGACACCAGAGAACATAGTATTTTATAGTGAGAATGTGGA
CAGAGCTGGCCGGAAAACAAAATTGGTATATGTTGTTGGTAAGAATGGCAGTGGATCTCTGAAAT
AG
Sequence 2: wild type murine OBP amino acid sequence
MVKFLLIAIALGVSCAHHESLDISPSEVNGDWHTLYIAADKVEKVKMNGDLRAYFEHMECNDDCG
TLKVKFHVQMNGKCQTHTVVGEKQEDGRYTTDCEYKFEVVMKEDGALFFHNVNVDESGQETNVIL
VAGKGETLSKAQKQELGKLVKEYNIPKENIQHLAPTGFKTVVLIWALQTDGPWKTIAIAADNVDK
IEISGEDKIEISGELRLYFHQITCEKECKKMNVTFYVNENGQCSLTTITGYLQDDGNTYRSQFQG
DNHYATVRTTPENIVFYSENVDRAGRKTKLVYVVGKNGSGSLK
Sequence 3: wild type rat OBP nucleic acid sequence (1f)
GAATCCAGGCTCTAACATGGTGAAGTTTCTGCTGATTGTTCTTGCATTAGGTGTATCCTGTGCAC
ATCATGAAAATCTTGATATCAGTCCCTCAGAGGTTAATGGGGACTGGCGCACCCTTTACATAGTT
GCAGATAATGTGGAGAAGGTAGCAGAAGGTGGATCCCTGAGAGCTTACTTTCAGCACATGGAATG
TGGTGATGAATGCCAGGAACTCAAAATCATATTCAATGTCAAGTTGGACAGTGAATGTCAGACAC
ACACTGTTGTGGGACAAAAACATGAAGATGGGCGGTACACTACTGACTACTCTGGTAGAAATTAC
TTCCATGTTTTGAAGAAGACAGATGACATTATTTTCTTTCACAACGTTAATGTCGATGAGAGTGG
AAGGAGACAATGTGATTTAGTTGCTGGGAAAAGAGAGGACCTGAACAAAGCACAGAAGCAGGAGC
TTAGGAAGCTGGCTGAGGAGTATAATATTCCAAATGAGAATACCCAGCACTTGGTGCCCACAGAC
ACTTGTAACCAATAAAGACTCCATATGGCTTCACAAAGGACAGCAAGGTCAGCAATATTTCCCAC
ATCACCTTTTCCATGAAATCAGAATCGTGACAATGAAGATAACTCATCCTTTTCTTATTTTTTCT
TTTCATCTTTCCTATGAAGCCAGAAAATCTGCTTCGTGGATTTGTTTCCCACCCTCCTATCATGG
TACTGATTCTTCTGTTGATAAAATAAATTTATTTTTCATGCAC
Sequence 4: wild type rat OBP nucleic acid sequence (2B)
ACACACTTCCAGGGTGAGCTGCCTTGTGTGAGAGCCCAGTGACTGGAGATGAAGAGCCGGCTCCT
CACCGTCCTGCTGCTGGGGCTGATGGCTGTCCTGAAGGCTCAGGAAGCCCCACCTGATGACCAGG
AGGATTTCTCTGGGAAGTGGTACACAAAGGCCACGGTTTGTGACAGGAACCACACAGATGGGAAG
AGACCTATGAAAGTGTTCCCTATGACTGTGACAGCCCTGGAAGGAGGGGACTTAGAGGTCCGGAT
AACATTCCGGGGGAAGGGTCATTGTCATTTGAGACGAATTACGATGCACAAAACTGATGAGCCTG
GCAAGTACACTACCTTCAAAGGCAAGAAGACCTTCTATACTAAGGAGATTCCTGTAAAGGACCAC
TACATCTTCTACATTAAAGGCCAGCGCCATGGGAAATCATATCTGAAGGGGAAACTCGTGGGGAG
AGACTCTAAGGACAACCCAGAGGCCATGGAGGAATTCAAGAAATTTGTAAAGAGCAAGGGATTCA
GAGAAGAAAACATTACTGTCCCTGAGCTGTTGGATGAGTGTGTACCTGGGAGTGACTAGGCACAG
CTGCCCGTCAGGATAGAGTTGCTGATCCTGCCCTAATGCTGACTCAGTTCTGATACATCCTGGGA
GCTCCCGAACTCCAGACGACTTTCCTCACCTTCATGGATGGACTTCCCTTCCACCTCAGCTTCAC
CCACCCCAGCACAGCTT
Sequence 5: wild type rat OBP nucleic acid sequence (OBP3)
TGGGCACCATCAGCAGAGAGATTGTCCCGACAGAGAGGCAATTCTATTCCCTACCAACATGAAGC
TGTTGCTGCTGCTGCTGTGTCTGGGCCTGACCCTGGTCTGTGGCCATGCAGAAGAAGCTAGTTTC
GAGAGAGGGAACCTCGATGTGGACAAGCTCAATGGGGATTGGTTTTCTATTGTCGTGGCCTCTGA
TAAAAGAGAAAAGATAGAAGAGAACGGCAGCATGAGAGTTTTTGTGCAGCACATCGATGTCTTGG
AGAATTCCTTAGGCTTCACGTTCCGTATTAAGGAAAATGGAGTGTGCACAGAATTTTCTTTGGTT
GCCGACAAAACAGCAAAGGATGGCGAATATTTTGTTGAGTATGACGGAGAAAATACATTTACTAT
ACTGAAGACAGACTATGACAATTATGTCATGTTTCATCTCGTTAATGTCAACAACGGGGAAACAT
TCCAGCTGATGGAGCTCTACGGCAGAACAAAGGATCTGAGTTCAGACATCAAGGAAAAGTTTGCA
AAACTATGTGTGGCACATGGAATCACTAGGGACAATATCATTGACCTAACCAAGACTGATCGCTG
TCTCCAGGCCCGAGGTTGAAGAAAGGCCTGAGCCTCCAGATTGCAGGGCAAGATCTATTTCTTCA
TCCTTTGTTCTATACAATAGAGTGCCTCTCTGTCCAGAAGTCAATCCAAGAAGTGCTTAATGGGT
TCCTTTATTCTTTCTTCCTGGATTACTCCGTGCTGAGTGGAGACTTCTCACCAGGACTCCAGCAT
TACCATTTCCTGTCCATGGAGCATCCTGAGACAAATTCTGCGATCTGATTTCCATCCTGTCTCAC
AGAAAAGTGCAATCCTGGTCTCTCCAGCATCTTCCCTAGTTACCCAGGACAACACATCGAGAATT
AAAAGCTTTCTTAAATTTCTCTTTGCCCCACTCATGATCATTCCGCACAAATTTCTTGCTCTTGC
AGTGCATAAATGATTACCCTTGCACTT
Sequence 6: wild type rat OBP amino acid sequence (1f)
MVKFLLIVLALGVSCAHHENLDISPSEVNGDWRTLYIVADNVEKVAEGGSLRAYFQHMECGDECQ
ELKIIFNVKLDSECQTHTVVGQKHEDGRYTTDYSGRNYFHVLKKTDDIIFFHNVNVDESGRRQCD
LVAGKREDLNKAQKQELRKLAEEYNIPNENTQHLVPTDTCNQ
Sequence 7: wild type rat OBP amino acid sequence (2B)
MKSRLLTVLLLGLMAVLKAQEAPPDDQEDFSGKWYTKATVCDRNHTDGKRPMKVFPMTVTALEGG
DLEVRITFRGKGHCHLRRITMHKTDEPGKYTTFKGKKTFYTKEIPVKDHYIFYIKGQRHGKSYLK
GKLVGRDSKDNPEAMEEFKKFVKSKGFREENITVPELLDECVPGSD
Sequence 8: wild type rat OBP amino acid sequence (OBP3)
MKLLLLLLCLGLTLVCGHAEEASFERGNLDVDKLNGDWFSIVVASDKREKIEENGSMRVFVQHID
VLENSLGFTFRIKENGVCTEFSLVADKTAKDGEYFVEYDGENTFTILKTDYDNYVMFHLVNVNNG
ETFQLMELYGRTKDLSSDIKEKFAKLCVAHGITRDNIIDLTKTDRCLQARG
Sequence 9: wild type human OBP nucleic acid sequence
CGCCCAGTGACCTGCCGAGGTCGGCAGCACAGAGCTCTGGAGATGAAGACCCTGTTCCTGGGTGT
CACGCTCGGCCTGGCCGCTGCCCTGTCCTTCACCCTGGAGGAGGAGGATATCACAGGGACCTGGT
ACGTGAAGGCCATGGTGGTCGATAAGGACTTTCCGGAGGACAGGAGGCCCAGGAAGGTGTCCCCA
GTGAAGGTGACAGCCCTGGGCGGTGGGAACTTGGAAGCCACGTTCACCTTCATGAGGGAGGATCG
GTGCATCCAGAAGAAAATCCTGATGCGGAAGACGGAGGAGCCTGGCAAATTCAGCGCCTATGGGG
GCAGGAAGCTCATATACCTGCAGGAGCTGCCCGGGACGGACGACTACGTCTTTTACTGCAAAGAC
CAGCGCCGTGGGGGCCTGCGCTACATGGGAAAGCTTGTGGCATCTGCTCCCTGCAGGGCCGTGCC
GCTGTCCCCACGTCGGCTCACCTGGCCACCTCACCTGCAGGTAGGAATCCTAATACCAACCTGGA
GGCCCTGGAAGAATTTAAGAAATTGGTGCAGCACAAGGGACTCTCGGAGGAGGACATTTTCATGC
CCCTGCAGACGGGAAGCTGCGTTCTCGAACACTAGGCAGCCCCCGGGTCTGCACCTCCAGAGCCC
ACCCTACCACCAGACACAGAGCCCGGACCACCTGGACCTACCCTCCAGCCATGACCCTTCCCTGC
TCCCACCCACCTGACTCCAAATAAAG
Sequence 10: wild type human OBP nucleic acid sequence
CGCCCAGTGACCTGCCGAGGTCGGCAGCACAGAGCTCTGGAGATGAAGACCCTGTTCCTGGGTGT
CACGCTCGGCCTGGCCGCTGCCCTGTCCTTCACCCTGGAGGAGGAGGATATCACAGGGACCTGGT
ACGTGAAGGCCATGGTGGTCGATAAGGACTTTCCGGAGGACAGGAGGCCCAGGAAGGTGTCCCCA
GTGAAGGTGACAGCCCTGGGCGGTGGGAAGTTGGAAGCCACGTTCACCTTCATGAGGGAGGATCG
GTGCATCCAGAAGAAAATCCTGATGCGGAAGACGGAGGAGCCTGGCAAATACAGCGCCTGCTTGT
CCGCAGTCGAGATGGACCAGATCACGCCTGCCCTCTGGGAGGCCCTAGCCATTGACACATTGAGG
AAGCTGAGGATTGGGACAAGGAGGCCAAGGATTAGATGGGGGCAGGAAGCTCATGTACCTGCAGG
AGCTGCCCAGGAGGGACCACTACATCTTTTACTGCAAAGACCAGCACCATGGGGGCCTGCTCCAC
ATGGGAAAGCTTGTGGGTAGGAATTCTGATACCAACCGGGAGGCCCTGGAAGAATTTAAGAAATT
GGTGCAGCGCAAGGGACTCTCGGAGGAGGACATTTTCACGCCCCTGCAGACGGGAAGCTGCGTTC
CCGAACACTAGGCAGCCCCCGGGTCTGCACCTCCAGAGCCCACCCTACCACCAGACACAGAGCCC
GGACCACCTGGACCTACCCTCCAGCCATGACCCTTCCCTGCTCCCACCCACCTGACTCCAAATAA
AG
Sequence 11: wild type human OBP nucleic acid sequence
CGAGGTCGGCAGCACAGAGCTCTGGAGATGAAGACCCTGTTCCTGGGTGTCACGCTCGGCCTGGC
CGCTGCCCTGTCCTTCACCCTGGAGGAGGAGGATATCACAGGGACCTGGTACGTGAAGGCCATGG
TGGTCGATAAGGACTTTCCGGAGGACAGGAGGCCCAGGAAGGTGTCCCCAGTGAAGGTGACAGCC
CTGGGCGGTGGGAACTTGGAAGCCACGTTCACCTTCATGAGGGAGGATCGGTGCATCCAGAAGAA
AATCCTGATGCGGAAGACGGAGGAGCCTGGCAAATTCAGCGCCTATGGGGGCAGGAAGCTCATAT
ACCTGCAGGAGCTGCCCGGGACGGACGACTACGTCTTTTACTGCAAAGACCAGCGCCGTGGGGGC
CTGCGCTACATGGGAAAGCTTGTGGGTAGGAATCCTAATACCAACCTGGAGGCCCTGGAAGAATT
TAAGAAATTGGTGCAGCACAAGGGACTCTCGGAGGAGGACATTTTCATGCCCCTGCAGACGGGAA
GCTGCGTTCTCGAACACTAGGCAGCCCCCGGGTCTGCACCTCCAGAGCCCACCCTACCACCAGAC
ACAGA
sequence 12: wild type human OBP amino acid sequence
MKTLFLGVTLGLAAALSFTLEEEDITGTWYVKAMVVDKDFPEDRRPRKVSPVKVTALGGGNLEAT
FTFMREDRCIQKKILMRKTEEPGKFSAYGGRKLIYLQELPGTDDYVFYCKDQRRGGLRYMGKLVA
SAPCRAVPLSPRRLTWPPHLQVGILIPTWRPWKNLRNWCSTRDSRRRTFSCPCRREAAFSNTRQP
PGLHLQSPPYHQTQSPDHLDLPSSHDPSLLPPT
sequence 13: wild type human OBP amino acid sequence (2B)
MKTLFLGVTLGLAAALSFTLEEEDITGTWYVKAMVVDKDFPEDRRPRKVSPVKVTALGGGKLEAT
FTFMREDRCIQKKILMRKTEEPGKYSACLSAVEMDQITPALWEALAIDTLRKLRIGTRRPRIRWG
QEAHVPAGAAQEGPLHLLLQRPAPWGPAPHGKACG
sequence 14: wild type human OBP amino acid sequence (2A)
MKTLFLGVTLGLAAALSFTLEEEDITGTWYVKAMVVDKDFPEDRRPRKVSPVKVTALGGGNLEAT
FTFMREDRCIQKKILMRKTEEPGKFSAYGGRKLIYLQELPGTDDYVFYCKDQRRGGLRYMGKLVG
RNPNTNLEALEEFKKLVQHKGLSEEDIFMPLQTGSCVLEH
Sequence 15. wild type bovine OBP amino acid sequence
AQEEEAEQNLSELSGPWRTVYIGSTNPEKIQENGPFRTYFRELVFDDEKGTVDFYFSVKRDGKWK
NVHVKATKQDDGTYVADYEGQNVFKIVSLSRTHLVAHNINVDKHGQTTELTELFVKLNVEDEDLE
KFWKLTEDKGIDKKNVVNFLENEDHPHPE
Sequence 16 wild type pig OBP nucleic acid sequence
ATGAAGAGTCTGCTGCTGAGTCTGGTCCTTGGTCTGGTTTGTGCCCAGGAACCTCAACCTGAACA
AGATCCCTTTGAGCTTTCAGGAAAATGGATAACCAGCTACATAGGCTCTAGTGACCTGGAGAAGA
TTGGAGAAAATGCACCCTTCCAGGTTTTCATGCGTAGCATTGAATTTGATGACAAAGAGAGCAAA
GTATACTTGAACTTTTTTAGCAAGGAAAATGGAATCTGTGAAGAATTTTCGCTGATCGGAACCAA
ACAAGAAGGCAATACTTACGATGTTAACTACGCAGGTAACAACAAATTTGTAGTTAGTTATGCGT
CCGAAACTGCCCTGATAATCTCTAACATCAATGTGGATGAAGAAGGCGACAAAACCATAATGACG
GGACTGTTGGGCAAAGGAACTGACATTGAAGACCAAGATTTGGAGAAGTTTAAAGAGGTGACAAG
AGAGAACGGGATTCCAGAAGAAAATATTGTGAACATCATCGAAAGAGATGACTGTCCTGCCAAGT
GA
Sequence 17 wild type pig OBP amino acid sequence
MKSLLLSLVLGLVCAQEPQPEQDPFELSGKWITSYIGSSDLEKIGENAPFQVFMRSIEFDDKESK
VYLNFFSKENGICEEFSLIGTKQEGNTYDVNYAGNNKFVVSYASETALIISNINVDEEGDKTIMT
GLLGKGTDIEDQDLEKFKEVTRENGIPEENIVNIIERDDCPAK
Sequence 18: wild type bovine albumin nucleic acid sequence
(ALB Bos Taurus 1)
ATGAAGTGGGTGACTTTTATTTCTCTTCTCCTTCTCTTCAGCTCTGCTTATTCCAGGGGTGTGTT
TCGTCGAGATACACACAAGAGTGAGATTGCTCATCGGTTTAAAGATTTGGGAGAAGAACATTTTA
AAGGCCTGGTACTGATTGCCTTTTCTCAGTATCTCCAGCAGTGTCCATTTGATGAGCATGTAAAA
TTAGTGAACGAACTAACTGAGTTTGCAAAAACATGTGTTGCTGATGAGTCCCATGCCGGCTGTGA
AAAGTCACTTCACACTCTCTTTGGAGATGAATTGTGTAAAGTTGCATCCCTTCGTGAAACCTATG
GTGACATGGCTGACTGCTGTGAGAAACAAGAGCCTGAAAGAAATGAATGCTTCCTGAGCCACAAA
GATGATAGCCCAGACCTCCCTAAATTGAAACCAGACCCCAATACTTTGTGTGATGAGTTTAAGGC
AGATGAAAAGAAGTTTTGGGGAAAATACCTATACGAAATTGCTAGAAGACATCCCTACTTTTATG
CACCAGAACTCCTTTACTATGCTAATAAATATAATGGAGTTTTTCAAGAATGCTGCCAAGCTGAA
GATAAAGGTGCCTGCCTGCTACCAAAGATTGAAACTATGAGAGAAAAAGTACTGACTTCATCTGC
CAGACAGAGACTCAGGTGTGCCAGTATTCAAAAATTTGGAGAAAGAGCTTTAAAAGCATGGTCAG
TAGCTCGCCTGAGCCAGAAATTTCCCAAGGCTGAGTTTGTAGAAGTTACCAAGCTAGTGACAGAT
CTCACAAAAGTCCACAAGGAATGCTGCCATGGTGACCTACTTGAATGCGCAGATGACAGGGCAGA
TCTTGCCAAGTACATATGTGATAATCAAGATACAATCTCCAGTAAACTGAAGGAATGCTGTGATA
AGCCTTTGTTGGAAAAATCCCACTGCATTGCTGAGGTAGAAAAAGATGCCATACCTGAAAACCTG
CCCCCATTAACTGCTGACTTTGCTGAAGATAAGGATGTTTGCAAAAACTATCAGGAAGCAAAAGA
TGCCTTCCTGGGCTCGTTTTTGTATGAATATTCAAGAAGGCATCCTGAATATGCTGTCTCAGTGC
TATTGAGACTTGCCAAGGAATATGAAGCCACACTGGAGGAATGCTGTGCCAAAGATGATCCACAT
GCATGCTATTCCACAGTGTTTGACAAACTTAAGCATCTTGTGGATGAGCCTCAGAATTTAATTAA
ACAAAACTGTGACCAATTCGAAAAACTTGGAGAGTATGGATTCCAAAATGCGCTCATAGTTCGTT
ACACCAGGAAAGTACCCCAAGTGTCAACTCCAACTCTCGTGGAGGTTTCAAGAAGCCTAGGAAAA
GTGGGTACTAGGTGTTGTACAAAGCCGGAATCAGAAAGAATGCCCTGTACTGAAGACTATCTGAG
CTTGATCCTGAACCGGTTGTGCGTGCTGCATGAGAAGACACCAGTGAGTGAAAAAGTCACCAAGT
GCTGCACAGAGTCATTGGTGAACAGACGGCCATGTTTCTCTGCTCTGACACCTGATGAAACATAT
GTACCCAAAGCCTTTGATGAGAAATTGTTCACCTTCCATGCAGATATATGCACACTTCCCGATAC
TGAGAAACAAATCAAGAAACAAACTGCACTTGTTGAGCTGTTGAAACACAAGCCCAAGGCAACAG
AGGAACAACTGAAAACCGTCATGGAGAATTTTGTGGCTTTTGTAGACAAGTGCTGTGCAGCTGAT
GACAAAGAAGCCTGCTTTGCTGTGGAGGGTCCAAAACTTGTTGTTTCAACTCAAACAGCCTTAGC
CTAA
Sequence 19: wild type bovine albumin amino acid sequence
(ALB Bos Taurus 1)
MKWVTFISLLLLFSSAYSRGVFRRDTHKSEIAHRFKDLGEEHFKGLVLIAFSQYLQQCPFDEHVK
LVNELTEFAKTCVADESHAGCEKSLHTLFGDELCKVASLRETYGDMADCCEKQEPERNECFLSHK
DDSPDLPKLKPDPNTLCKEFKADEKKFWGKYLYEIARRHPYFYAPELLYYANKYNGVFQECCQAE
DKGACLLPKIETMREKVLTSSARQRLRCASIQKFGERALKAWSVARLSQKFPKAEFVEVTKLVTD
LTKVHKECCHGDLLECADDRADLAKYICDNQDTISSKLKECCDKPLLEKSHCIAEVEKDAIPENL
PPLTADFAEDKDVCKNYQEAKDAFLGSFLYEYSRRHPEYAVSVLLRLAKEYEATLEECCAKDDPH
ACYSTVFDKLKHLVDEPQNLIKQNCDQFEKLGEYGFQNALIVRYTRKVPQVSTPTLVEVSRSLGK
VGTRCCTKPESERMPCTEDYLSLILNRLCVLHEKTPVSEKVTKCCTESLVNRRPCFSALTPDETY
VPKAFDEKLFTFHADICTLPDTEKQIKKQTALVELLKHKPKATEEQLKTVMENFVAFVDKCCAAD
DKEACFAVEGPKLVVSTQTALA
Sequence 20: wild type human albumin nucleic acid sequence
(ALB Homo Sapiens 1)
AGCTTTTCTCTTCTGTCAACCCCACACGCCTTTGGCACAATGAAGTGGGTAACCTTTATTTCCCT
TCTTTTTCTCTTTAGCTCGGCTTATTCCAGGGGTGTGTTTCGTCGAGATGCACACAAGAGTGAGG
TTGCTCATCGGTTTAAAGATTTGGGAGAAGAAAATTTCAAAGCCTTGGTGTTGATTGCCTTTGCT
CAGTATCTTCAGCAGTGTCCATTTGAAGATCATGTAAAATTAGTGAATGAAGTAACTGAATTTGC
AAAAACATGTGTTGCTGATGAGTCAGCTGAAAATTGTGACAAATCACTTCATACCCTTTTTGGAG
ACAAATTATGCACAGTTGCAACTCTTCGTGAAACCTATGGTGAAATGGCTGACTGCTGTGCAAAA
CAAGAACCTGAGAGAAATGAATGCTTCTTGCAACACAAAGATGACAACCCAAACCTCCCCCGATT
GGTGAGACCAGAGGTTGATGTGATGTGCACTGCTTTTCATGACAATGAAGAGACATTTTTGAAAA
AATACTTATATGAAATTGCCAGAAGACATCCTTACTTTTATGCCCCGGAACTCCTTTTCTTTGCT
AAAAGGTATAAAGCTGCTTTTACAGAATGTTGCCAAGCTGCTGATAAAGCTGCCTGCCTGTTGCC
AAAGCTCGATGAACTTCGGGATGAAGGGAAGGCTTCGTCTGCCAAACAGAGACTCAAGTGTGCCA
GTCTCCAAAAATTTGGAGAAAGAGCTTTCAAAGCATGGGCAGTAGCTCGCCTGAGCCAGAGATTT
CCCAAAGCTGAGTTTGCAGAAGTTTCCAAGTTAGTGACAGATCTTACCAAAGTCCACACGGAATG
CTGCCATGGAGATCTGCTTGAATGTGCTGATGACAGGGCGGACCTTGCCAAGTATATCTGTGAAA
ATCAAGATTCGATCTCCAGTAAACTGAAGGAATGCTGTGAAAAACCTCTGTTGGAAAAATCCCAC
TGCATTGCCGAAGTGGAAAATGATGAGATGCCTGCTGACTTGCCTTCATTAGCTGCTGATTTTGT
TGAAAGTAAGGATGTTTGCAAAAACTATGCTGAGGCAAAGGATGTCTTCCTGGGCATGTTTTTGT
ATGAATATGCAAGAAGGCATCCTGATTACTCTGTCGTGCTGCTGCTGAGACTTGCCAAGACATAT
GAAACCACTCTAGAGAAGTGCTGTGCCGCTGCAGATCCTCATGAATGCTATGCCAAAGTGTTCGA
TGAATTTAAACCTCTTGTGGAAGAGCCTCAGAATTTAATCAAACAAAATTGTGAGCTTTTTGAGC
AGCTTGGAGAGTACAAATTCCAGAATGCGCTATTAGTTCGTTACACCAAGAAAGTACCCCAAGTG
TCAACTCCAACTCTTGTAGAGGTCTCAAGAAACCTAGGAAAAGTGGGCAGCAAATGTTGTAAACA
TCCTGAAGCAAAAAGAATGCCCTGTGCAGAAGACTATCTATCCGTGGTCCTGAACCAGTTATGTG
TGTTGCATGAGAAAACGCCAGTAAGTGACAGAGTCACCAAATGCTGCACAGAATCCTTGGTGAAC
AGGCGACCATGCTTTTCAGCTCTGGAAGTCGATGAAACATACGTTCCCAAAGAGTTTAATGCTGA
AACATTCACCTTCCATGCAGATATATGCACACTTTCTGAGAAGGAGAGACAAATCAAGAAACAAA
CTGCACTTGTTGAGCTCGTGAAACACAAGCCCAAGGCAACAAAAGAGCAACTGAAAGCTGTTATG
GATGATTTCGCAGCTTTTGTAGAGAAGTGCTGCAAGGCTGACGATAAGGAGACCTGCTTTGCCGA
GGAGGGTAAAAAACTTGTTGCTGCAAGTCAAGCTGCCTTAGGCTTATAACATCTACATTTAAAAG
CATCTCAGCCTACCATGAGAATAAGAGAAAGAAAATGAAGATCAAAAGCTTATTCATCTGTTTTC
TTTTTCGTTGGTGTAAAGCCAACACCCTGTCTAAAAAACATAAATTTCTTTAATCATTTTGCCTC
TTTTCTCTGTGCTTCAATTAATAAAAAATGGAAAGAATCTAATAGAGTGGTACAGCACTGTTATT
TTTCAAAGATGTGTTGCTATCCTGAAAATTCTGTAGGTTCTGTGGAAGTTCCAGTGTTCTCTCTT
ATTCCACTTCGGTAGAGGATTTCTAGTTTCTGTGGGCTAATTAAATAAATCACTAATACTCTTCT
AAGTT
Sequence 21: wild type human albumin amino acid sequence
(ALB Homo Sapiens 1)
MKWVTFISLLFLFSSAYSRGVFRRDAHKSEVAHRFKDLGEENFKALVLIAFAQYLQQCPFEDHVK
LVNEVTEFAKTCVADESAENCDKSLHTLFGDKLCTVATLRETYGEMADCCAKQEPERNECFLQHK
DDNPNLPRLVRPEVDVMCTAFHDNEETFLKKYLYEIARRHPYFYAPELLFFAKRYKAAFTECCQA
ADKAACLLPKLDELRDEGKASSAKQRLKCASLQKFGERAFKAWAVARLSQRFPKAEFAEVSKLVT
DLTKVHTECCHGDLLECADDRADLAKYICENQDSISSKLKECCEKPLLEKSHCIAEVENDEMPAD
LPSLAADFVESKDVCKNYAEAKDVFLGMFLYEYARRHPDYSVVLLLRLAKTYETTLEKCCAAADP
HECYAKVFDEFKPLVEEPQNLIKQNCELFEQLGEYKFQNALLVRYTKKVPQVSTPTLVEVSRNLG
KVGSKCCKHPEAKRMPCAEDYLSVVLNQLCVLHEKTPVSDRVTKCCTESLVNRRPCFSALEVDET
YVPKEFNAETFTFHADICTLSEKERQIKKQTALVELVKHKPKATKEQLKAVMDDFAAFVEKCCKA
DDKETCFAEEGKKLVAASQAALGL
Sequence 22: wild type wild boar albumin nucleic acid sequence
(ALB Sus scrofa 1)
ACCTTTTCTCTTCTATCAACCCCACAAGCCTTTGGCACAATGAAGTGGGTGACTTTTATTTCCCT
TCTCTTTCTCTTCAGCTCTGCTTATTCCAGGGGTGTGTTTCGTCGAGATACATACAAGAGTGAAA
TTGCTCATCGGTTTAAAGATTTGGGAGAACAATATTTCAAAGGCCTAGTGCTGATTGCCTTTTCT
CAGCATCTCCAGCAATGCCCATATGAAGAGCATGTGAAATTAGTGAGGGAAGTAACTGAGTTTGC
AAAAACATGTGTTGCTGATGAGTCAGCTGAAAATTGTGACAAGTCAATTCACACTCTCTTTGGAG
ATAAATTATGTGCAATTCCATCCCTTCGTGAACACTATGGTGACTTGGCTGACTGCTGTGAAAAA
GAAGAGCCTGAGAGAAACGAATGCTTCCTCCAACACAAAAATGATAACCCCGACATCCCTAAATT
GAAACCAGACCCTGTTGCTTTATGCGCTGACTTCCAGGAAGATGAACAGAAGTTTTGGGGAAAAT
ACCTATATGAAATTGCCAGAAGACATCCCTATTTCTACGCCCCAGAACTCCTTTATTATGCCATT
ATATATAAAGATGTTTTTTCAGAATGCTGCCAAGCTGCTGATAAAGCTGCCTGCCTGTTACCAAA
GATTGAGCATCTGAGAGAAAAAGTACTGACTTCCGCCGCCAAACAGAGACTTAAGTGTGCCAGTA
TCCAAAAATTCGGAGAGAGAGCTTTCAAAGCATGGTCATTAGCTCGCCTGAGCCAGAGATTTCCC
AAGGCTGACTTTACAGAGATTTCCAAGATAGTGACAGATCTTGCAAAAGTCCACAAGGAATGCTG
CCATGGTGACCTGCTTGAATGTGCAGATGACAGGGCGGATCTTGCCAAATATATATGTGAAAATC
AAGACACAATCTCCACTAAACTGAAGGAATGCTGTGATAAGCCTCTGTTGGAAAAATCCCACTGC
ATTGCTGAGGCAAAAAGAGATGAATTGCCTGCAGACCTGAACCCATTAGAACATGATTTTGTTGA
AGATAAGGAAGTTTGTAAAAACTATAAAGAAGCAAAGCATGTCTTCCTGGGCACGTTTTTGTATG
AGTATTCAAGAAGGCACCCAGACTACTCTGTCTCATTGCTGCTGAGAATTGCCAAGATATATGAA
GCCACACTGGAGGACTGCTGTGCCAAAGAGGATCCTCCGGCATGCTATGCCACAGTGTTTGATAA
ATTTCAGCCTCTTGTGGATGAGCCTAAGAATTTAATCAAACAAAACTGTGAACTTTTTGAAAAAC
TTGGAGAGTATGGATTCCAAAATGCGCTCATAGTTCGTTACACCAAGAAAGTACCCCAAGTGTCA
ACTCCAACTCTTGTGGAGGTCGCAAGAAAACTAGGACTAGTGGGCTCTAGGTGTTGTAAGCGTCC
TGAAGAAGAAAGACTGTCCTGTGCTGAAGACTATCTGTCCCTGGTCCTGAACCGGTTGTGCGTGT
TGCACGAGAAGACACCAGTGAGCGAAAAAGTTACCAAATGCTGCACAGAGTCCTTGGTGAACAGA
CGGCCTTGCTTTTCTGCTCTGACACCAGACGAAACATACAAACCCAAAGAATTTGTTGAGGGAAC
CTTCACCTTCCATGCAGACCTATGCACACTTCCTGAGGATGAGAAACAAATCAAGAAGCAAACTG
CACTCGTTGAGTTGTTGAAACACAAGCCTCATGCAACAGAGGAACAACTGAGAACTGTCCTGGGC
AACTTTGCAGCCTTTGTACAAAAGTGCTGCGCCGCTCCTGACCATGAGGCCTGCTTTGCTGTGGA
GGGTCCGAAATTTGTTATTGAAATTCGAGGGATCTTAGCCTAAACAACACAGTGACAAGCATCTC
AGACTACCCTGAGAATAAGAGAAAGAGAAATGAAGACCTAGACTTATCCATCTCTTTTTCTTTTC
TGTTGGTTTTAAACCAACACCCTGTCTAAAGTACACAAATTTCTTTAAATATTTTGCCTCTTTTC
TCTGTGCTACAATTAATAAAAAAATGAAAAGAATCT
Sequence 23: wild type wild boar albumin amino acid sequence
(ALB Sus scrofa 1)
MKWVTFISLLFLFSSAYSRGVFRRDTYKSEIAHRFKDLGEQYFKGLVLIAFSQHLQQCPYEEHVK
LVREVTEFAKTCVADESAENCDKSIHTLFGDKLCAIPSLREHYGDLADCCEKEEPERNECFLQHK
NDNPDIPKLKPDPVALCADFQEDEQKFWGKYLYEIARRHPYFYAPELLYYAIIYKDVFSECCQAA
DKAACLLPKIEHLREKVLTSAAKQRLKCASIQKFGERAFKAWSLARLSQRFPKADFTEISKIVTD
LAKVHKECCHGDLLECADDRADLAKYICENQDTISTKLKECCDKPLLEKSHCIAEAKRDELPADL
NPLEHDFVEDKEVCKNYKEAKHVFLGTFLYEYSRRHPDYSVSLLLRIAKIYEATLEDCCAKEDPP
ACYATVFDKFQPLVDEPKNLIKQNCELFEKLGEYGFQNALIVRYTKKVPQVSTPTLVEVARKLGL
VGSRCCKRPEEERLSCAEDYLSLVLNRLCVLHEKTPVSEKVTKCCTESLVNRRPCFSALTPDETY
KPKEFVEGTFTFHADLCTLPEDEKQIKKQTALVELLKHKPHATEEQLRTVLGNFAAPVQKCCAAP
DHEACFAVEGPKFVIEIRGILA
Sequence 24: wild type rabbit albumin nucleic acid sequence
(ALB Oryctolagus cuniculus 1)
ATTCAATATAAAGAAGGGTTTGGACATCTTTCTCCTACTGGTACCACGGAATTTTGGCACAATGA
AGTGGGTAACCTTTATCTCCCTTCTTTTCCTCTTCAGCTCTGCTTATTCCAGGGGTGTGTTTCGC
CGAGAAGCACATAAAAGTGAGATTGCTCATCGGTTTAATGATGTGGGAGAAGAACATTTCATAGG
CCTGGTGCTGATTACCTTTTCTCAGTATCTCCAGAAGTGCCCATATGAAGAGCATGCGAAGTTAG
TGAAGGAAGTAACAGACTTGGCAAAAGCATGTGTTGCTGATGAGTCAGCAGCAAATTGTGACAAA
TCACTTCATGATATTTTTGGAGACAAAATCTGTGCATTGCCAAGTCTTCGTGACACCTATGGTGA
CGTGGCTGACTGCTGTGAGAAAAAAGAACCTGAGCGAAACGAATGCTTCCTGCACCACAAGGATG
ATAAACCCGACTTGCCTCCGTTTGCGAGACCAGAAGCTGATGTTTTGTGCAAAGCCTTTCATGAT
GATGAAAAGGCATTCTTTGGACACTATTTATATGAAGTTGCCAGAAGACATCCTTACTTTTATGC
CCCTGAACTCCTTTACTATGCTCAGAAGTACAAAGCCATTCTAACAGAATGTTGCGAAGCTGCTG
ATAAAGGGGCCTGCCTCACACCTAAGCTTGATGCTTTGGAAGGAAAAAGCCTGATTTCAGCTGCC
CAAGAGAGACTCAGGTGTGCCAGTATTCAGAAATTTGGAGACAGAGCTTACAAAGCATGGGCACT
TGTTCGTCTGAGCCAAAGATTTCCCAAGGCTGACTTCACAGACATTTCCAAGATAGTGACAGATC
TCACCAAAGTCCACAAGGAATGCTGCCACGGTGACCTGCTTGAATGTGCAGATGACAGGGCGGAC
CTTGCCAAGTACATGTGTGAACATCAGGAAACAATCTCCAGTCATCTGAAGGAATGCTGTGATAA
GCCAATATTGGAAAAAGCCCACTGCATTTATGGTTTGCATAATGATGAGACACCTGCTGGCTTGC
CAGCAGTAGCTGAGGAATTTGTTGAGGATAAGGATGTTTGCAAAAATTATGAAGAGGCAAAAGAT
CTCTTCTTGGGCAAGTTTTTGTATGAGTATTCAAGAAGGCACCCTGATTACTCTGTCGTTCTGCT
GCTGAGACTTGGCAAGGCCTATGAAGCCACCCTGAAAAAGTGCTGTGCCACTGATGACCCTCACG
CATGCTATGCCAAAGTGCTTGATGAGTTTCAGCCTCTTGTGGATGAACCCAAGAATTTAGTGAAA
CAAAACTGTGAACTCTATGAGCAGCTTGGTGACTACAACTTCCAAAATGCGCTCCTAGTTCGTTA
TACCAAGAAAGTACCTCAAGTGTCAACTCCAACTCTCGTGGAAATATCAAGAAGCCTAGGAAAAG
TGGGCAGCAAGTGCTGTAAGCATCCTGAAGCAGAAAGACTGCCTTGTGTTGAAGATTATCTGTCC
GTGGTCCTGAACAGGTTGTGCGTGTTGCATGAGAAGACACCAGTGAGTGAGAAAGTCACCAAATG
CTGCTCAGAGTCATTGGTCGACAGACGACCATGCTTTAGCGCCCTGGGCCCCGATGAAACATACG
TCCCCAAAGAATTTAATGCTGAAACATTCACCTTCCATGCGGACATATGCACTCTTCCAGAAACG
GAGAGGAAAATCAAGAAACAAACGGCACTTGTTGAGTTGGTGAAACACAAGCCCCACGCAACAAA
TGATCAACTGAAAACTGTTGTTGGAGAGTTCACAGCTTTGTTAGACAAGTGCTGCAGTGCTGAAG
ACAAGGAGGCCTGCTTTGCTGTGGAGGGTCCAAAACTTGTTGAATCAAGTAAAGCTACCTTAGGC
TAAAAAATCACAGCCACAATAATCTCAGCCTACCCTGAGAATAAGAGAAGAGAAATGAAGACCCA
GAGCCTATTCATCTGTTTTTCTTTTCTGTTGATATAAAACCAACAG
Sequence 25: wild type rabbit albumin amino acid sequence
(ALB Oryctolagus cuniculus 1)
MKWVTFISLLFLFSSAYSRGVFRREAHKSEIAHRFNDVGEEHFIGLVLITFSQYLQKCPYEEHAK
LVKEVTDLAKACVADESAANCDKSLHDIFGDKICALPSLRDTYGDVADCCEKKEPERNECFLHHK
DDKPDLPPFARPEADVLCKAFHDDEKAFFGHYLYEVARRHPYFYAPELLYYAQKYKAILTECCEA
ADKGACLTPKLDALEGKSLISAAQERLRCASIQKFGDRAYKAWALVRLSQRFPKADFTDISKIVT
DLTKVHKECCHGDLLECADDRADLAKYMCEHQETISSHLKECCDKPILEKAHCIYGLHNDETPAG
LPAVAEEFVEDKDVCKNYEEAKDLFLGKFLYEYSRRHPDYSVVLLLRLGKAYEATLKKCCATDDP
HACYAKVLDEFQPLVDEPKNLVKQNCELYEQLGDYNFQNALLVRYTKKVPQVSTPTLVEISRSLG
KVGSKCCKHPEAERLPCVEDYLSVVLNRLCVLHEKTPVSEKVTKCCSESLVDRRPCFSALGPDET
YVPKEFNAETFTFHADICTLPETERKIKKQTALVELVKHKPHATNDQLKTVVGEFTALLDKCCSA
EDKEACFAVEGPKLVESSKATLG
Sequence 26: wild type mouse albumin nucleic acid sequence
(ALB Mus Musculus 1)
ATGAAGTGGGTAACCTTTCTCCTCCTCCTCTTCGTCTCCGGCTCTGCTTTTTCCAGGGGTGTGTT
TCGCCGAGAAGCACACAAGAGTGAGATCGCCCATCGGTATAATGATTTGGGAGAACAACATTTCA
AAGGCCTAGTCCTGATTGCCTTTTCCCAGTATCTCCAGAAATGCTCATACGATGAGCATGCCAAA
TTAGTGCAGGAAGTAACAGACTTTGCAAAGACGTGTGTTGCCGATGAGTCTGCCGCCAACTGTGA
CAAATCCCTTCACACTCTTTTTGGAGATAAGTTGTGTGCCATTCCAAACCTCCGTGAAAACTATG
GTGAACTGGCTGACTGCTGTACAAAACAAGAGCCCGAAAGAAACGAATGTTTCCTGCAACACAAA
GATGACAACCCCAGCCTGCCACCATTTGAAAGGCCAGAGGCTGAGGCCATGTGCACCTCCTTTAA
GGAAAACCCAACCACCTTTATGGGACACTATTTGCATGAAGTTGCCAGAAGACATCCTTATTTCT
ATGCCCCAGAACTTCTTTACTATGCTGAGCAGTACAATGAGATTCTGACCCAGTGTTGTGCAGAG
GCTGACAAGGAAAGCTGCCTGACCCCGAAGCTTGATGGTGTGAAGGAGAAAGCATTGGTCTCATC
TGTCCGTCAGAGAATGAAGTGCTCCAGTATGCAGAAGTTTGGAGAGAGAGCTTTTAAAGCATGGG
CAGTAGCTCGTCTGAGCCAGACATTCCCCAATGCTGACTTTGCAGAAATCACCAAATTGGCAACA
GACCTGACCAAAGTCAACAAGGAGTGCTGCCATGGTGACCTGCTGGAATGCGCAGATGACAGGGC
GGAACTTGCCAAGTACATGTGTGAAAACCAGGCGACTATCTCCAGCAAACTGCAGACTTGCTGCG
ATAAACCACTGTTGAAGAAAGCCCACTGTCTTAGTGAGGTGGAGCATGACACCATGCCTGCTGAT
CTGCCTGCCATTGCTGCTGATTTTGTTGAGGACCAGGAAGTGTGCAAGAACTATGCTGAGGCCAA
GGATGTCTTCCTGGGCACGTTCTTGTATGAATATTCAAGAAGACACCCTGATTACTCTGTATCCC
TGTTGCTGAGACTTGCTAAGAAATATGAAGCCACTCTGGAAAAGTGCTGCGCTGAAGCCAATCCT
CCCGCATGCTACGGCACAGTGCTTGCTGAATTTCAGCCTCTTGTAGAAGAGCCTAAGAACTTGGT
CAAAACCAACTGTGATCTTTACGAGAAGCTTGGAGAATATGGATTCCAAAATGCCATTCTAGTTC
GCTACACCCAGAAAGCACCTCAGGTGTCAACCCCAACTCTCGTGGAGGCTGCAAGAAACCTAGGA
AGAGTGGGCACCAAGTGTTGTACACTTCCTGAAGATCAGAGACTGCCTTGTGTGGAGGACTATCT
GTCTGCAATCCTGAACCGTGTGTGTCTGCTGCATGAGAAGACCCCAGTGAGTGAGCATGTTACCA
AGTGCTGTAGTGGATCCCTGGTGGAAAGGCGGCCATGCTTCTCTGCTCTGACAGTTGATGAAACA
TATGTCCCCAAAGAGTTTAAAGCTGAGACCTTCACCTTCCACTCTGATATCTGCACACTTCCAGA
GAAGGAGAAGCAGATTAAGAAACAAACGGCTCTTGCTGAGCTGGTGAAGCACAAGCCCAAGGCTA
CAGCGGAGCAACTGAAGACTGTCATGGATGACTTTGCACAGTTCCTGGATACATGTTGCAAGGCT
GCTGACAAGGACACCTGCTTCTCGACTGAGGGTCCAAACCTTGTCACTAGATGCAAAGACGCCTT
AGCCTAAACACACCACAACCACAACCTTCTCAGGCTACCCTGACACATGAAAGGGCGAATTCCAG
CACACTGGCGGCCGTTACTAGTGGATCCGAGCTCG
Sequence 27: wild type mouse albumin amino acid sequence
(ALB Mus Musculus 1)
MKWVTFLLLLFVSGSAFSRGVFRREAHKSEIAHRYNDLGEQHFKGLVLIAPSQYLQKCSYDEHAK
LVQEVTDFAKTCVADESAANCDKSLHTLFGDKLCAIPNLRENYGELADCCTKQEPERNECFLQHK
DDNPSLPPFERPEAEAMCTSFKENPTTFMGHYLHEVARRHPYFYAPELLYYAEQYNEILTQCCAE
ADKESCLTPKLDGVKEKALVSSVRQRMKCSSMQKFGERAFKAWAVARLSQTFPNADFAEITKLAT
DLTKVNKECCHGDLLECADDRAELAKYMCENQATISSKLQTCCDKPLLKKAHCLSEVEHDTMPAD
LPAIAADFVEDQEVCKNYAEAKDVFLGTFLYEYSRRHPDYSVSLLLRLAKKYEATLEKCCAEANP
PACYGTVLAEFQPLVEEPKNLVKTNCDLYEKLGEYGFQNAILVRYTQKAPQVSTPTLVEAARNLG
RVGTKCCTLPEDQRLPCVEDYLSAILNRVCLLHEKTPVSEHVTKCCSGSLVERRPCFSALTVDEY
VPKEFKAETFTFHSDICTLPEKEKQIKKQTALAELVKHKPKATAEQLKTVMDDFAQFLDTCCKAA
DKDTCFSTEGPNLVTRCKDALA
Sequence 28: wild type rat albumin nucleic acid sequence
(ALB Rattus Norvegicus 1)
ATGAAGTGGGTAACCTTTCTCCTCCTCCTCTTCATCTCCGGTTCTGCCTTTTCTAGGGGTGTGTT
TCGCCGAGAAGCACACAAGAGTGAGATCGCCCATCGGTTTAAGGACTTAGGAGAACAGCATTTCA
AAGGCCTAGTCCTGATTGCCTTTTCCCAGTATCTCCAGAAATGCCCATATGAAGAGCATATCAAA
TTGGTGCAGGAAGTAACAGACTTTGCAAAAACATGTGTCGCTGATGAGAATGCCGAAAACTGTGA
CAAGTCCATTCACACTCTCTTCGGAGACAAGTTATGCGCCATTCCAAAGCTTCGTGACAACTACG
GTGAACTGGCTGACTGCTGTGCAAAACAAGAGCCCGAAAGAAACGAGTGTTTCCTGCAGCACAAG
GATGACAACCCCAACCTGCCACCCTTCCAGAGGCCGGAGGCTGAGGCCATGTGCACCTCCTTCCA
GGAGAACCCTACCAGCTTTCTGGGACACTATTTGCATGAAGTTGCCAGGAGACATCCTTATTTCT
ATGCCCCAGAACTCCTTTACTATGCTGAGAAATACAATGAGGTTCTGACCCAGTGCTGCACAGAG
TCTGACAAAGCAGCCTGCCTGACACCGAAGCTTGATGCCGTGAAAGAGAAAGCACTGGTCGCAGC
TGTCCGTCAGAGGATGAAGTGCTCCAGTATGCAGAGATTTGGAGAGAGAGCCTTCAAAGCCTGGG
CAGTAGCTCGTATGAGCCAGAGATTCCCCAATGCTGAGTTCGCAGAAATCACCAAATTGGCAACA
GACGTTACCAAAATCAACAAGGAGTGCTGTCACGGCGACCTGTTGGAATGCGCGGATGACAGGGC
AGAACTTGCCAAGTACATGTGTGAGAACCAGGCCACTATCTCCAGCAAACTGCAGGCTTGCTGTG
ATAAGCCAGTGCTGCAGAAATCCCAGTGTCTCGCTGAGACAGAACATGACAACATTCCTGCCGAT
CTGCCCTCAATAGCTGCTGACTTTGTTGAGGATAAGGAAGTGTGTAAGAACTATGCTGAGGCCAA
GGATGTCTTCCTGGGCACGTTTTTGTATGAATATTCAAGAAGGCACCCCGATTACTCCGTGTCCC
TGCTGCTGAGACTTGCTAAGAAATATGAAGCCACACTGGAGAAGTGCTGTGCTGAAGGCGATCCT
CCTGCCTGCTACGGCACAGTGCTTGCAGAATTTCAGCCTCTTGTAGAAGAACCTAAGAACTTGGT
CAAAACTAACTGTGAGCTTTACGAGAAGCTTGGAGAGTATGGATTCCAAAACGCCGTTCTGGTTC
GATACACCCAGAAAGCACCTCAGGTGTCGACCCCAACTCTCGTGGAGGCAGCAAGAAACCTGGGA
AGAGTGGGCACCAAGTGTTGTACCCTTCCTGAAGCTCAGAGACTGCCCTGTGTGGAAGACTATCT
GTCTGCCATCCTGAACCGTCTGTGTGTGCTGCATGAGAAGACCCCAGTGAGCGAGAAGGTCACCA
AGTGCTGTAGTGGGTCCTTGGTGGAAAGACGGCCATGTTTCTCTGCTCTGACAGTTGACGAGACA
TATGTCCCCAAAGAGTTTAAAGCTGAGACCTTCACCTTCCACTCTGATATCTGCACACTCCCAGA
CAAGGAGAAGCAGATAAAGAAGCAAACGGCTCTCGCTGAGCTGGTGAAACACAAGCCCAAGGCCA
CAGAAGATCAGCTGAAGACGGTGATGGGTGACTTCGCACAATTCGTGGACAAGTGTTGCAAGGCT
GCCGACAAGGATAACTGCTTCGCCACTGAGGGGCCAAACCTTGTTGCTAGAAGCAAAGAAGCCTT
AGCCTAAACACATCACAACCATCTCAGGCTACCCTGAGAAAAAAAGACATGAAGACTCAGGACTC
ATCTCTTCTGTTGGTGTAAAACCAACACCCTAAGGAACACAAATTTCTTTGAACATTTGACTTCT
TTTCTC
Sequence 29: wild type rat albumin amino acid sequence
(ALB Rattus Norvegicus 1)
MKWVTFLLLLFISGSAFSRGVFRREAHKSEIAHRFKDLGEQHFKGLVLIAFSQYLQKCPYEEHIK
LVQEVTDFAKTCVADENAENCDKSIHTLFGDKLCAIPKLRDNYGELADCCAKQEPERNECFLQHK
DDNPNLPPFQRPEAEAMCTSFQENPTSFLGHYLHEVARRHPYFYAPELLYYAEKYNEVLTQCCTE
SDKAACLTPKLDAVKEKALVAAVRQRMKCSSMQRFGERAFKAWAVARMSQRFPNAEFAEITKLAT
DVTKINKECCHGDLLECADDRAELAKYMCENQATISSKLQACCDKPVLQKSQCLAETEHDNIPAD
LPSIAADFVEDKEVCKNYAEAKDVFLGTFLYEYSRRHPDYSVSLLLRLAKKYEATLEKCCAEGDP
PACYGTVLAEFQPLVEEPKNLVKTNCELYEKLGEYGFQNAVLVRYTQKAPQVSTPTLVEAARNLG
RVGTKCCTLPEAQRLPCVEDYLSAILNRLCVLHEKTPVSEKVTKCCSGSLVERRPCFSALTVDET
YVPKEFKAETFTFHSDICTLPDKEKQIKKQTALAELVKHKPKATEDQLKTVMGDFAQFVDKCCKA
ADKDNCFATEGPNLVARSKEALA
Sequence 30: Bos taurus von Ebner minor salivary gland protein
(C20orf114), mRNA
GCGTGTCCAGGTTCTGCCACGTGCCACCTGCCGACCCTGAAGAAGATGGCCTACCCGTGGACCTT
CACCTTCCTCTGTGGTTTGCTGGCAGCCAACCTGGTAGGAGCCACCTTAAGCCCTCCTGTGGTTC
TCAGTCTCAGCACAGAAGTCATCAAGCAAATGCTGGCTCAGAAACTGAAGAATCACGATGTTACC
AACACCCTGCAGCAGCTGCCACTGCTCACTGCCATGGAGGAGGAGTCGTCCAGGGGCATTTTCGG
CAACCTGGTGAAATCCATCCTGAAGCATATTCTCTGGATGAAAGTCACCTCAGCCAGCATCGGTC
AGCTGCAGGTGCAGCCCCTGGCCAACGGCCGGCAGCTGATGGTCAAGGCCCCCCTGGACGTGGTG
GCTGGATTCAACGTGCCCCTTTTCAAGACCGTTGTGGAGCTGCATGTGGAGGTGGAGGCCCAAGC
CATCATCCACGTGGAGACTAGGGAGAAGGACCACGCCCGCCTGGTCCTCAGCGAGTGCTCCAACA
CCGGCGGGAGCCTGCGCGTCAGCCTGCTGCACAAGCTCTCCTTCCTGCTCAAATGCTTAGCCGAC
AAGGTCATAAGCCTTCTGACGCCAGCGCCCCCTAAACTGGTGAAAAGCGAGCTGTGTCCTGTGCT
CAAGGCGGGCTTTGAGGACATGCGTGGGGAACTCTTGAATCTGACGAAGGTGCCCATGTCTCTCA
ACTCTGAGCACCTGAAGCTTGATTTTATTTCTCCTGTCATCGACCACAGTGTTGTCCATCTCATC
CTGGGGGCCAGGTTGTTCAACTCAGAAGGAAAGGTGACTAAGCTGTTCAATGTTGCTGGGGATTC
CCTGAATCTGCCCACCCTGAACCAGACCCCTTTCAGGCTCACCGTGAGGAAGGATGTGGTGGTCG
CTATCATAGCTGCCTTGATCCATTCGGGAAAACTCACAGTCCTGTTGGACTATGTGCTTCCTGAG
GTAGCCCGCCAGCTGAGGTCAAGCATCAAGGTGATCGACGAAACGGCAGCAGCGCAGCTGGGGCC
CACACAGATCGTGAAGATCATGAGTCAGACGACCCCAATGCTCATTCTGGACCAGGGCAATGCCA
AGGTGGCCCAACTGATCGTGCTGGAAATATTCGCCACCGATAAAGACAGCCGCCCCCTCTTCACC
CTGGGCATCGAAGCCTCCTCGGACATTCAGTTTTACGTCGAAGATGGCCTACTTGTGTTCAGCTT
TAACGAAATCAGAGCTGATCGGATCCATCTGATGAACTCAGACATCGGTGTGTTCAACCCTAAGC
TTCTGAACAACATCACCACCAAGATCCTCACCTCCATCCTGCTGCCAAACGAGAATGGCAAATTA
AGATCTGGGATCCCAGTGTCAATGGTGAAAAACTTGGGATTTAAGTCGATTTCATTGTCTCTGAC
CAAGGAAGCCCTTGTGGTCACCCAAGCCTCCTCTTAGAACCTCAGCCCACTTTCCTCTCTCCCAG
TGAAGACTTGCACTGTGGTCCTCCAGGGAAGGCTGTGTCTCAATGAGAGTGTGGGAGCCAGCGCT
GTAATCTGTCCCTCCCTACAATGAATAAACTTTGTGAATCTTGCAGTCCAAAAAAAAAAAAAAA
Sequence 31: Von Ebner minor salivary gland protein [Bos taurus]
MAYPWTFTFLCGLLAANLVGATLSPPVVLSLSTEVIKQMLAQKLKNHDVTNTLQQLPLLTAMEEE
SSRGIFGNLVKSILKHILWMKVTSASIGQLQVQPLANGRQLMVKAPLDVVAGFNVPLFKTVVELH
VEVEAQAIIHVETREKDHARLVLSECSNTGGSLRVSLLHKLSFLLKCLADKVISLLTPAPPKLVK
SELCPVLKAGFEDMRGELLNLTKVPMSLNSEHLKLDFISPVIDHSVVHLILGARLFNSEGKVTKL
FNVAGDSLNLPTLNQTPFRLTVRKDVVVAIIAALIHSGKLTVLLDYVLPEVARQLRSSIKVIDET
AAAQLGPTQIVKIMSQTTPMLILDQGNAKVAQLIVLEIFATDKDSRPLFTLGIEASSDIQFYVED
GLLVFSFNEIRADRIHLMNSDIGVFNPKLLNNITTKILTSILLPNENGKLRSGIPVSMVKNLGFK
SISLSLTKEALVVTQASS
Sequence 32: Sus scrofa von Ebner gland protein mRNA,
complete cds
ATGATGAGGGCTCTGCTCCTGGCCATTGGCCTCGGCCTCGTTGCTGCCCTGCAGGCCCAGGAGTT
CCCGGCCGTGGGGCAGCCGCTGCAGGATCTGCTGGGGAGATGGTATCTGAAGGCCATGACCTCGG
ACCCGGAGATTCCCGGGAAGAAGCCCGAGTCGGTGACCCCCCTGATTCTCAAGGCCCTGGAGGGG
GGCGACCTGGAAGCCCAGATAACCTTTCTGATTGACGGTCAGTGCCAGGACGTGACACTGGTCCT
AAAGAAAACCAACCAGCCCTTCACGTTCACGGCCTATGACGGCAAGCGCGTGGTGTACATCTTAC
CGTCCAAGGTGAAGGACCACTACATTCTCTACTGCGAGGGTGAGCTGGACGGGCAGGAGGTCCGC
ATGGCGAAGCTCGTGGGAAGAGACCCAGAGAACAACCCAGAGGCTTTGGAGGAGTTCAAGGAGGT
GGCAAGAGCCAAAGGGCTAAACCTGGACATCGTCAGGCCCCAGCAAAGCGAAACCTGCTCTCCAG
GAGGGAACTAG
Sequence 33: Von Ebner gland protein [Sus scrofa]
MMRALLLAIGLGLVAALQAQEFPAVGQPLQDLLGRWYLKAMTSDPEIPGKKPESVTPLILKALEG
GDLEAQITFLIDGQCQDVTLVLKKTNQPFTFTAYDGKRVVYILPSKVKDHYILYCEGELDGQEVR
MAKLVGRDPENNPEALEEFKEVARAKGLNLDIVRPQQSETCSPGGN
Sequence 34: Human putative von Ebner gland protein Nucleic acid
sequence (C20orf114)
GCCCGGGAGAGGAGAGGAGCGGGCCGAGGACTCCAGCGTGCCCAGATGGCCGGCCCGTGGACCTT
CACCCTTCTCTGTGGTTTGCTGGCAGCCACCTTGATCCAAGCCACCCTCAGTCCCACTGCAGTTC
TCATCCTCGGCCCAAAAGTCATCAAAGAAAAGCTGACACAGGAGCTGAAGGACCACAACGCCACC
AGCATCCTGCAGCAGcTGCCGCTGCTCAGTGCCATGCGGGAAAAGCCAGCCGGAGGCATCCCTGT
GCTGGGCAGCCTGGTGAACACCGTCCTGAAGCACATCATCTGGCTGAAGGTCATCACAGCTAACA
TCCTCCAGCTGCAGGTGAAGCCCTCGGCCAATGACCAGGAGCTGCTAGTCAAGATCCCCCTGGAC
ATGGTGGCTGGATTCAACACGCCCCTGGTCAAGACCATCGTGGAGTTCCACATGACGACTGAGGC
CCAAGCCACCATCCGCATGGACACCAGTGCAAGTGGCCCCACCCGCCTGGTCCTCAGTGACTGTG
CCACCAGCCATGGGAGCCTGCGCATCCAACTGCTGCATAAGCTCTCcTTCCTGGTGAACGCCTTA
GCTAAGCAGGTCATGAACCTCCTAGTGCCATCCCTGCCCAATCTAGTGAAAAACCAGCTGTGTCC
CGTGATCGAGGCTTCCTTCAATGGCATGTATGCAGACCTCCTGCAGCTGGTGAAGGTGCCCATTT
CCCTCAGCATTGACCGTCTGGAGTTTGACCTTCTGTATCCTGCCATCAAGGGTGACACCATTCAG
CTCTACCTGGGGGCCAAGTTGTTGGACTCACAGGGAAAGGTGACCAAGTGGTTCAATAACTCTGC
AGCTTCCCTGACAATGCCCACCCTGGACAACATCCCGTTCAGCCTCATCGTGAGTCAGGACGTGG
TGAAAGCTGCAGTGGCTGCTGTGCTCTCTCCAGAAGAATTCATGGTCCTGTTGGACTCTGTGCTT
CCTGAGAGTGCCCATCGGCTGAAGTCAAGCATCGGGCTGATCAATGAAAAGGCTGCAGATAAGCT
GGGATCTACCCAGATCGTGAAGATCCTAACTCAGGACACTCCCGAGTTTTTTATAGACCAAGGCC
ATGCCAAGGTGGCCCAACTGATCGTGCTGGAAGTGTTTCCCTCCAGTGAAGCCCTCCGCCCTTTG
TTCACCCTGGGCATCGAAGCCAGCTCGGAAGCTCAGTTTTACACCAAAGGTGACCAACTTATACT
CAACTTGAATAACATCAGCTCTGATCGGATCCAGCTGATGAACTCTGGGATTGGCTGGTTCCAAC
CTGATGTTCTGAAAAACATCATCACTGAGATCATCCACTCCATCCTGCTGCCGAACCAGAATGGC
AAATTAAGATCTGGGGTCCCAGTGTCATTGGTGAAGGCCTTGGGATTCGAGGCAGCTGAGTCCTC
ACTGACCAAGGATGCCCTTGTGCTTACTCCAGCCTCCTTGTGGAAACCCAGCTCTCCTGTCTCCC
AGTGAAGACTTGGATGGCAGCCATCAGGGAAGGCTGGGTCCCAGCTGGGAGTATGGGTGTGAGCT
CTATAGACCATCCCTCTCTGCAATCAATAAACACTTGCCTGTGATGCCTGC
Sequence 35: Human putative von Ebner gland protein Amino acid
sequence (C20orf114)
MAGPWTFTLLCGLLAATLIQATLSPTAVLILGPKVIKEKLTQELKDHNATSILQQLPLLSAMREK
PAGGIPVLGSLVNTVLKHIIWLKVITANILQLQVKPSANDQELLVKIPLDMVAGFNTPLVKTIVE
FHMTTEAQATIRMDTSASGPTRLVLSDCATSHGSLRIQLLHKLSFLVNALAKQVMNLLVPSLPNL
VKNQLCPVIEASFNGMYADLLQLVKVPISLSIDRLEFDLLYPAIKGDTIQLYLGAKLLDSQGKVT
KWFNNSAASLTMPTLDNIPFSLIVSQDVVKAAVAAVLSPEEPMVLLDSVLPESAHRLKSSIGLIN
EKAADKLGSTQIVKILTQDTPEFFIDQGHAKVAQLIVLEVFPSSEALRPLFTLGIEASSEAQFYT
KGDQLILNLNNISSDRIQLMNSGIGWFQPDVLKNIITEIIHSILLPNQNGKLRSGVPVSLVKALG
FEAAESSLTKDALVLTPASLWKPSSPVSQ
Sequence 36: Mus musculus major urinary protein 1 (Mup1), mRNA
CTGAACCCAGAGAGTATATAAGAACAAGCAAAGGGGCTGGGGAGTGGAGTGTAGCCACGATCACA
AGAAAGACGTGGTCCTGACAGACAGACAATCCTATTCCCTACCAAAATGAAGATGCTGCTGCTGC
TGTGTTTGGGACTGACCCTAGTCTGTGTCCATGCAGAAGAAGCTAGTTCTACGGGAAGGAACTTT
AATGTAGAAAAGATTAATGGGGAATGGCATACTATTATCCTGGCCTCTGACAAAAGAGAAAAGAT
AGAAGATAATGGCAACTTTAGACTTTTTCTGGAGCAAATCCATGTCTTGGAGAATTCCTTAGTTC
TTAAATTCCATACTGTAAGAGATGAAGAGTGCTCGGAATTATCTATGGTTGCTGACAAAACAGAA
AAGGCTGGTGAATATTCTGTGACGTATGATGGATTCAATACATTTACTATACCTAAGACAGACTA
TGATAACTTTCTTATGGCTCATCTCATTAACGAAAAGGATGGGGAAACCTTCCAGCTGATGGGGC
TCTATGGCCGAGAACCAGATTTGAGTTCAGACATCAAGGAAAGGTTTGCACAACTATGTGAGAAG
CATGGAATCCTTAGAGAAAATATCATTGACCTATCCAATGCCAATCGCTGCCTCCAGGCCCGAGA
ATGAAGAATGGCCTGAGCCTCCAGTGTTGAGTGGAGACTTCTCACCAGGACTCCACCATCATCCC
TTCCTATCCATACAGCATCCCCAGTATAAATTCTGTGATCTGCATTCCATCCTGTCTCACTGAGA
AGTCCAATTCCAGTCTATCCACATGTTACCTAGGATACCTCATCAAGAATCAAAGACTTCTTTAA
ATTTTTCTTTGATATACCCATGACAATTTTTCATGAATTTCTTCCTCTTCCTGTTCAATAAATGA
TTACCCTTGCACTTA
Sequence 37: major urinary protein 1 [Mus musculus]
MKMLLLLCLGLTLVCVHAEEASSTGRNFNVEKINGEWHTIILASDKREKIEDNGNFRLFLEQIHV
LENSLVLKFHTVRDEECSELSMVADKTEKAGEYSVTYDGFNTFTIPKTDYDNFLMAHLINEKDGE
TFQLMGLYGREPDLSSDIKERFAQLCEKHGILRENIIDLSNANRCLQARE
Sequence 38: Helicoverpa assulta pheromone binding protein (pbp)
mRNA, complete cds
ATGAATTTTGCTAAGCCCTTAGAAGACTGTAAGAAAGAGATGGATCTCCCAGACTCGGTGACGAC
AGACTTCTACAACTTCTGGAAGGAAGGCTACGAGTTCACGAACAGACAGACGGGCTGCGCCATCC
TCTGCCTCTCCTCCAAGCTAGAGCTGCTGGACCAGGAGATGAAGCTGCACCACGGCAAGGCGCAG
GAGTTCGCCAAGAAACATGGCGCTGACGATGCTATGGCTAAGCAGCTGGTAGACCTGATCCACGG
CTGCTCGCGGTCTACTCCTGACGTGACAGACGATCCCTGTATGAAGGCCCTCAACGTGGCCAAGT
GCTTCAAGGCCAAGATACACGAGCTCAACTGGGCGCCCAGCATGGACCTCGTCGTCGGAGAAGTC
TTGGCCGAAGTTTAG
Sequence 39: pheromone binding protein [Helicoverpa assulta]
MNFAKPLEDCKKEMDLPDSVTTDFYNFWKEGYEFTNRQTGCAILCLSSKLELLDQEMKLHHGKAQ
EFAKKHGADDAMAKQLVDLIHGCSRSTPDVTDDPCMKALNVAKCFKAKIHELNWAPSMDLVVGEV
LAEV
Sequence 40: Sesamia nonagrioides pheromone binding protein 1
precursor (PBP1) mRNA, complete cds
ATTATTCAAAATGGCTGATTCAAGATGGTGGTTCGCGAGTTTCATCTGCGTCATTATTATGACAA
GTTCGGTGATGTCTTCCAAGGAGTTGGTCTCCAAAATGAGTTCCGGGTTCTCGAAGGTTTTGGAT
CAGTGTAAAGCTGAGCTGAACGTGGGCGAACACATAATGCAAGACATGTACAACTTCTGGCGCGA
GGAGTACGAGCTGGTGAACCGCGACCTGGGATGCATGGTGATGTGCATGGCCTCCAAGTTGGACC
TGGTAGGAGACGACCAGAAGATGCACCATGGAAAGGCCGAGGAGTTTGCCAAGAGTCATGGAGCT
GATGACGAGCTGGCTAAGCAGCTGGTGGGCATCATCCATGCCTGCGAGACGCAGCACCAAGCCAT
CGAGGATCCCTGCAGCCGCACGCTGGAGGTGGCCAAGTGCTTCCGCTCGAAGATGCACGAGCTGA
AGTGGGCCCCGCCCATGGAGGTCGCCATAGAAGAGATTATGACAGCTGTTTAGGTGGAATATGGG
ATAGAAAGGGGAGGAAGGAGTGAAATAGGGCCTTTTCAATTCTTATTTAAAAAATGTAATAATAA
TACTAAAGGTGCCGGTGGTTTATTAGTTTCTTATTGATTATAACTTATTATTACTAACATCTCTC
GCAACTCGTCAGTTTCTTATTATTATTTAATAATAACCGGTGTTAGAATTATTTTTATTAAAATA
AAGTATATTATTTTAGTCCAAAAAAAAAAAAAAAAAAAAAAAAAA
Sequence 41: pheromone binding protein 1 precursor
[Sesamia nonagrioides]
MADSRWWFASFICVIIMTSSVMSSKELVSKMSSGFSKVLDQCKAELNVGEHIMQDMYNFWREEYE
LVNRDLGCMVMCMASKLDLVGDDQKMHHGKAEEFAKSHGADDELAKQLVGIIHACETQHQAIEDP
CSRTLEVAKCFRSKMHELKWAPPMEVAIEEIMTAV
Sequence 42: Sesamia nonagrioides pheromone binding protein 2
precursor (PBP2) mRNA, complete cds
AATGGCGCTGCATCGATCGCCCATCATGTCGGCACGCTTGGCGCTGGTACTGATCGCCAGTCTGT
TCATCGTCGTGAAATGTTCTCAAGAAGTCATGAAGAATCTGACCCATCATTTCTCTAAGCCTTTG
GAAGACTGTAAGAAGGAGATGGACCTCCCGGACTCAGTGATCACAGATTTCTACAATTTCTGGAA
AGAAGGCTACGAGTTCACGAGCAGACATACAGGCTGTGCCATACTCTGCCTCTCATCTAAGCTGG
AACTGCTCGATCCAGACCTTAAGTTGCATCATGGAAAGGCGCAGGAGTTCGCGCAGAAACATGGC
GCTGACGAGGCCATGGCGAAGCAGCTGGTAGGCCTGATCCACGGCTGTATGGAGACAATCCGCGA
ACCGGCCGACGACCCCTGCGTGAGGGCTCAGAACGTAGTCATGTGCTTCAAGGCCAAGATACATG
AGCTGANCTGGGCGCCTAGCTTGGACCTCATCGTGGGAGAAGTCTTGGCTGAAGTCTAGCATGAT
GCCCTTGGTTCCGTGATATACCTTTATCTTCTCTTCGTCATAGAAGGCCATCATTGCATGTGATA
GTGATGTTGTTGTTTTGAATGCAAAACATAGTTTCATCTTTTTCATTTGTTTTGCTTGAGTGTTT
TCAGCTAGAGACATTACGTAAATCAAAGTCTTTTTTATCAAATATCATTCTCTGTTAAGAAACCA
ATAACCAGTGCTCAGACAACATTAATGTTATGTGCGGTTGTAATGTAATGCAATGCTTATGACCT
GCAGGAATAAATGCAAATAAGTTTATATCTACATTACATTATGTTTATACATTACAGTACATTGT
GTTATACAAGTCTGATTTGTTTCTATCTCTACTTTAACGACAAGGCTTGTTCAATGGACTACAGA
TATTTCTACAGTTAGTTATTTGATTAATATTTAATAATTCTTGTGAAGAGTCTCCTGTCTCGCCA
GTTCTCATCAAAGGGAGTGGGTACATTGTAAGTTGCAAGTTCTGGATGTCATATTAATAAAGAAT
ACATCTTTACAAAAAAAAAAAAAAAAAAAAAAAAAAAA
Sequence 43: pheromone binding protein 2 precursor
[Sesamia nonagrioides]
MALHRSPIMSARLALVLIASLFIVVKCSQEVMKNLTHHFSKPLEDCKKEMDLPDSVITDFYNFWK
EGYEFTSRHTGCAILCLSSKLELLDPDLKLHHGKAQEFAQKHGADEAMAKQLVGLIHGCMETIRE
PADDPCVRAQNVVMCFKAKIHELXWAPSLDLIVGEVLAEV
Sequence 44: Spodoptera exigua pheromone binding protein 1 (PBP1)
mRNA, complete cds
ACGCGGGGGCAGATAACAAGATGGCGGGCGCAAAATGGCGGTTTGTCTGTGTTGTGTTCGCGCTG
TACCTGACCAGCGCCGCGCTGGGCTCGCAGGAGCTCATGATGAAGATGACTAAGGGATTCACGAA
AGTCGTCGATGAGTGCAAAGCTGAGCTTAACGCGGGGGAGCACATCATGCAGGACATGTACAACT
ACTGGCGCGAAGACTACCAGCTCATTAACCGGGACTTGGGCTGCATGATCCTGTGCATGGCAAAG
AAGTTGGACCTCATGGAAGACCAGAAGATGCACCACGGGAAGACAGAAGAATTCGCCAAGAGTCA
TGGCGCTGATGACGAGGTTGCCAAGAAGCTGGTGAGCATAATCCACGAATGCGAGCAGCAGCACG
CCGGCATAGCGGACGATTGCATGAGGGTGTTGGAGATATCCAAATGCTTCCGCACCAAGATTCAC
GAGCTCAAATGGGCACCCAACATGGAGGTCATTATGGAAGAGGTGATGACCGCCGTGTAGACACG
AGGGAACCAGGAAACAATGTCATTTTAGGGAAAACTGCTGCAGTTGTTGGAGTGTCACGCGGGAT
AATGATCTGCAGCGTTAGCAAAACTGATGTACATACTTGTAATCGAGAATGCTATGGCAAACGAA
ACAAATGTATTTGGAGATTTATCAGTTTGAATACGTTGTGTGGCGCGTGGGCAATAGTGAATCTA
TACGTATGAACAACATTTGTTTCCTTTTATTTAGCGTTAACGATCACAAGTTGTACTGAACGATA
ACTAAAGCTCATAATGGTTCTAAGATTATCTCTAGATTGCAGGGCTATAACTGGAAAGGGTTTCG
TGTCATTTCGTTTCATGTCGCTGATTGATCAACACTTTCTAACACCTTTACACAATTCTCTTCAA
TCGTCGGAGTTATTCTACTTCACCCAGAAGTGAAATTGTTGTATCATTATCCTGGCTCTTTATTC
AGTGAAACTATGTAGCTGTATAAGTATTATTTATTTCCTCTTTAGGTTCTTGGTTAATTAAAGTG
TTTCAATTCATGAAAAAAAAAAAAAAAAAAAAAAAAA
Sequence 45: pheromone binding protein 1 [Spodoptera exigua]
MAGAKWRFVCVVFALYLTSAALGSQELMMKMTKGFTKVVDECKAELNAGEHIMQDMYNYWREDYQ
LINRDLGCMILCMAKKLDLMEDQKMHHGKTEEFAKSHGADDEVAKKLVSIIHECEQQHAGIADDC
MRVLEISKCFRTKIHELKWAPNMEVIMEEVMTAV
Sequence 46: Spodoptera exigua pheromone binding protein 2 (PBP2)
mRNA, complete cds
ACGCGGGGGACCATGTCGGTGAGGGTGGCGCTGGTGGTGGCCGCCAGTATGCTGGTAGTGGTACA
GGCGTCGCAAGATGTCATGAAGAACTTGGCCATCAATTTCGCGAAACCTTTGGATGACTGTAAGA
AGGAGATGGACCTGCCAGACTCGGTGACGACCGACTTCTACAACTTCTGGAAGGAAGGATACGAG
CTGACGAACAGACAGACCGGCTGTGCTATCCTGTGTCTCTCTTCGAAGTTGGAGATTCTTGACCA
AGAACTGAACCTGCATCACGGCAGGGCGCAGGAGTTTGCTATGAAACACGGCGCTGACGAGACCA
TGGCGAAGCAGATAGTGGACATGATCCACACTTGTGCGCAGTCTACTCCCGACGTAGCGGCGGAC
CCTTGCATGAAGACCCTGAATGTAGCCAAGTGCTTCAAGTTGAAGATACACGAGCTCAACTGGGC
GCCCAGCATGGAGCTCATCGTGGGAGAAGTGCTGGCTGAAGTGTAACTTGAATCACTCAAGACCT
TTAAGCTGGCCTTCATTATGTGAGGTCTTCATAAACATATCTTTGACGTCTCGGCTCGTTGAACG
GACCCCAGATTAGGTTAGGTTGGTCGTGAAGCATCTCCTGGCTCGCCAGTTCGCCTCAGAGGGTG
TGGGTAGTAGTAGGCTGCAGCAAGATGTCAAATATTGTTCAATATACTGTACATCATTAAAAAAA
Sequence 47: pheromone binding protein 2 [Spodoptera exigua]
MSVRVALVVAASMLVVVQASQDVMKNLAINFAKPLDDCKKEMDLPDSVTTDFYNFWKEGYELTNR
QTGCAILCLSSKLEILDQELNLHHGRAQEFAMKHGADETMAKQIVDMIHTCAQSTPDVAADPCMK
TLNVAKCFKLKIHELNWAPSMELIVGEVLAEV
Sequence 48: Drosophila melanogaster CG10436-PA (Pbprp1) mRNA,
complete cds
AGTTCAACTTTAGCAATTTTTGGGGAGAAGCAAAAATGGTTGCAAGGCATTTTAGTTTTTTTTTA
GCACTACTCATACTATATGATTTAATTCCTAGTAATCAAGGAGTGGAAATTAATCCTACGATCAT
AAAGCAGGTGAGAAAGCTGCGAATGCGATGCTTAAATCAGACAGGAGCTTCTGTAGATGTGATTG
ACAAGTCGGTGAAAAATAGAATACTACCTACAGATCCCGAGATCAAGTGTTTTCTCTACTGCATG
TTTGATATGTTCGGATTGATTGATTCACAAAACATAATGCACTTGGAAGCACTGTTGGAGGTTTT
ACCCGAGGAAATACACAAAACGATTAACGGATTAGTCAGTTCATGTGGAACTCAGAAGGGAAAAG
ATGGCTGTGATACCGCTTATGAAACCGTCAAGTGCTACATTGCTGTAAACGGAAAATTCATATGG
GAAGAGATAATAGTGCTACTTGGGTAGCGCTAACCAACCTAAATATATCCCGATCCACGATTCCC
AAGAGCAGCAAACAGCGCAGGATGCG
Sequence 49: CG10436-PA [Drosophila melanogaster]
MVARHFSFFLALLILYDLIPSNQGVEINPTIIKQVRKLRMRCLNQTGASVDVIDKSVKNRILPTD
PEIKCFLYCMFDMFGLIDSQNIMHLEALLEVLPEEIHKTINGLVSSCGTQKGKDGCDTAYETVKC
YIAVNGKFIWEEIIVLLG
Sequence 50: Drosophila melanogaster CG11421-PA (Pbprp3) mRNA,
complete cds
AAAGCAAATTCAATTGTGACTGCGGTTGTCAAACAATTCTTGCGTGTCGGGTGTGTGCAGTATCG
AGTTCTGGCCATAACTACTTCTGCTAAAAGCGAACGAGCTTGTTTTTGTTTTATTCAGAGCTCGC
AAATAAGGCCGAGCCAGGGCACAATTTTTGCTGTTTCACGGATGGACCAGGAAGGACCACGCAGC
AGCGGAAAGGAGCGAAACGGAAAGAGCCACATTAAAATGGCTTTGAATGGCTTTGGTCGGCGTGT
CAGTGCGTCTGTCCTTTTAATCGCCTTGTCGCTGCTCAGCGGAGCGCTGATCCTGCCGCCGGCTG
CGGCGCAGCGTGACGAGAACTATCCACCGCCGGGCATCCTGAAAATGGCCAAGCCCTTCCACGAC
GCGTGTGTGGAGAAGACGGGCGTAACCGAGGCTGCCATCAAGGAGTTCAGCGATGGGGAGATTCA
CGAGGACGAGAAGCTCAAATGCTACATGAACTGCTTCTTCCACGAGATCGAAGTGGTGGACGACA
ATGGGGACGTGCATCTGGAGAAGCTCTTCGCCACGGTACCGCTCTCCATGCGCGACAAGCTGATG
GAGATGTCCAAGGGCTGCGTCCATCCGGAGGGCGATACGCTGTGCCACAAGGCCTGGTGGTTCCA
CCAGTGCTGGAAAAAGGCCGATCCCAAGCACTACTTCTTGCCGTGAACACCTGGGCCACCTTTCA
GCCCAGTTCCAGTTCCATGGTCCGTGGACCACCCGTTGCCGACCCCGCTCTATTTATGTGGTAGT
TTAGTTTCTGCTAGTTTTCAATAGCTGTCGAGTAATAAACGTAGGCGAGTTGTGCATGCAAGCTA
A
Sequence 51: CG11421-PA [Drosophila melanogaster]
MALNGFGRRVSASVLLIALSLLSGALILPPAAAQRDENYPPPGILKMAKPFHDACVEKTGVTEAA
IKEFSDGEIHEDEKLKCYMNCFFHEIEVVDDNGDVHLEKLFATVPLSMRDKLMEMSKGCVHPEGD
TLCHKAWWFHQCWKKADPKHYFLP
Sequence 52: Drosophila melanogaster CG1668-PA, isoform A
(Pbprp2) mRNA, complete cds
TCACAATCACTCATCTCACCCAGAGCTGTTGATCGATTTAATTACAAGCGGGATTTCTCATCTCT
CATTTTGCATTTAGCATTTTGCATTTTCATTTCCATTTCCACTAGCCATAGCCATTCCCAATTCT
ATATCCCCGGCATTTGCAGCGATTTCATGCCAGTCACCAATTAAGCAGGTAAGTGGAGATCGGTG
GGCCATCTCATCTGGCAGCGGCAGTTCCAGCGGGGTGTCACTCGTTCACACGATGCCCAGTCGAG
GGCATCTCCGCCGGATTCCGTCCCATCCCGTCCAGAGCGGCGGAGTGAAGTGGAGTGCCATGTGC
CATGTGCTGCCCATGTAGTTCATAATTGCGCGTAATTGCCGGAGCTGCTTGAGACGCAGCTGGAG
ATCGGCGATGGATCCGATCTGCCAAATCAATCACGGGACTCGGCTTAGGCAATAGCTCCTATAAA
ACGCCGACGTTGCCGGCGATTCGCATCCAAGTCAGAGTTCGCACGTCGCGCAGTTCAATCGCAAA
TCGAAATGTCGCATCTGGTTCACCTGACCGTCCTGCTCCTAGTGGGCATCCTCTGCCTGGGCGCC
ACCAGCGCCAAGCCGCACGAGGAGATCAACAGGGACCACGCCGCCGAGCTGGCCAACGAGTGCAA
GGCTGAGACGGGAGCCACCGATGAGGATGTGGAGCAGCTGATGAGCCACGACCTGCCCGAGAGAC
ACGAGGCCAAGTGCCTGCGCGCCTGCGTGATGAAAAAGCTGCAGATAATGGATGAATCCGGTAAG
CTGAACAAGGAACACGCCATCGAGTTGGTCAAGGTCATGAGCAAGCACGATGCAGAGAAGGAAGA
CGCTCCCGCCGAGGTGGTGGCCAAGTGCGAGGCCATCGAGACACCCGAGGATCATTGCGACGCTG
CCTTCGCCTACGAGGAATGCATTTACGAGCAAATGAAGGAGCATGGACTCGAGCTGGAGGAGCAC
TGAGAACAGATTTGAGACCCATGACGACCCCCCGTTACTGTATCACAAGCGCCCTTCTGGAATAT
AACCATCTTTTTTTTTTATGTGTATACTATGAATTAAGTACTTGATAAACTGAGAAACTGCAGG
Sequence 53: CG1668-PA, isoform A [Drosophila melanogaster]
MSHLVHLTVLLLVGILCLGATSAKPHEEINRDHAAELANECKAETGATDEDVEQLMSHDLPERHE
AKCLRACVMKKLQIMDESGKLNKEHAIELVKVMSKHDAEKEDAPAEVVAKCEAIETPEDHCDAAF
AYEECIYEQMKEHGLELEEH
Sequence 54: Drosophila melanogaster CG1176-PA (Pbprp4) mRNA,
complete cds
AGCACTTTGTTTGTTCAAGATGTATTCCGCGTTAGTTAGAGCTTGTGCTGTCATTGCTTTTCTGA
TCTTGAGCCCGAATTGTGCCAGGGCTCTACAGGATCACGCCAAGGATAATGGTGATATTTTCATC
ATAAACTATGATAGTTTCGATGGCGATGTGGATGACATATCCACCACCACTTCAGCTCCTAGAGA
GGCTGACTACGTAGATTTTGACGAGGTTAATCGTAACTGCAATGCTAGTTTCATAACGTCGATGA
CCAATGTCTTGCAGTTTAATAACACTGGGGATTTGCCAGATGACAAGGATAAGGTAACCAGCATG
TGCTATTTTCACTGCTTTTTCGAAAAGTCCGGTTTGATGACGGACTATAAGTTAAATACGGATCT
GGTGCGCAAATATGTTTGGCCAGCCACTGGCGATTCCGTTGAGGCCTGCGAAGCTGAAGGCAAGG
ACGAGACGAATGCTTGCATGCGGGGCTATGCCATCGTCAAGTGCGTGTTTACTAGAGCCCTCACG
GATGCTAGAAACAAACCCACTGTATGAATAACATCAAAGGTCACATCTCGGACTTATCA
Sequence 55: CG1176-PA [Drosophila melanogaster]
MYSALVRACAVIAFLILSPNCARALQDHAKDNGDIFIINYDSFDGDVDDISTTTSAPREALYVDF
DEVNRNCNASFITSMTNVLQFNNTGDLPDDKDKVTSMCYFHCFFEKSGLMTDYKLNTDLVRKYVW
PATGDSVEACEAEGKDETNACMRGYAIVKCVFTRALTDARNKPTV
Sequence 56: Drosophila melanogaster CG6641-PA (Pbprp5) mRNA,
complete cds
AAGTTCCGTTCAGACACACCGACCTAGCATCATGCAGTCTACTCCAATCATTCTGGTGGCAATCG
TCCTTCTCGGCGCCGCACTGGTGCGAGCCTTTGACGAGAAGGAGGCCCTGGCCAAGCTGATGGAG
TCAGCCGAGAGCTGCATGCCGGAAGTGGGGGCCACCGATGCCGATCTGCAGGAAATGGTCAAGAA
GCAGCCAGCCAGCACATATGCCGGCAAGTGCCTGCGCGCCTGCGTGATGAAGAACATCGGAATTC
TGGACGCCAACGGAAAACTGGACACGGAGGCAGGTCACGAGAAGGCCAAGCAGTACACGGGCAAC
GATCCGGCCAAGCTAAAGATTGCCCTGGAGATCGGCGACACCTGTGCCGCCATCACTGTGCCGGA
TGATCACTGCGAGGCCGCCGAAGCCTATGGCACTTGCTTCAGGGGCGAGGCCAAGAAACATGGAC
TCTTGTAATCATTGATGCAGCGCTACCCACCTGGACACG
Sequence 57: CG6641-PA [Drosophila melanogaster]
MQSTPIILVAIVLLGAALVRAFDEKEALAKLMESAESCMPEVGATDADLQEMVKKQPASTYAGKC
LRACVMKNIGILDANGKLDTEAGHEKAKQYTGNDPAKLKIALEIGDTCAAITVPDDHCEAAEAYG
TCFRGEAKKHGLL

1. A method to identify and/or to confirm the binding and function of a volatile compound onto a membrane-integrated receptor comprising the steps of: —selecting a compound which may be a ligand for said receptor,
solubilizing said compound by airborne absorption onto a volatile-compound-Binding Protein (BP), making a BP-ligand-complex, applying said ligand complex on cells expressing said receptor, measuring the functional response of said receptor, and, —identifying a ligand for said receptor and/or confirming the binding and function of said compound onto said receptor.

2. The method according to claim 1, wherein the functional response may be measured by studying cellular signaling molecules chosen from the group: Ca2+, cAMP pool, IP3, GTP, melanophore assay and MAP-kinase; or; wherein the functional response may be measured by studying G protein/OR interaction, desensitization of the receptor; or; wherein the functional response may be measured by studying the modulation of a reporting system.

3. The method according to claim 1, wherein the functional response is measured using a radioisotope, fluorescent or luminescent method.

4. A method to identify and/or confirm the binding of a volatile compound onto a membrane-integrated receptor comprising the steps of: —selecting a compound which may be a ligand for said receptor, solubilizing said compound by airborne absorption onto volatile-compound-Binding Protein (BP), making a BP-ligand-complex,
applying said ligand complex to cell membranes comprising said receptor, measuring the binding of said compound to said receptor, and, —identifying a ligand for said receptor and/or confirming the binding of said compound onto said receptor.

5. The method according to claim 4, wherein the binding is measured using a radioisotope, fluorescent (FRET, polarization) or luminescent method (BRET).

6. The method according to any of claims 1, wherein said cells are heterologous cells; or according to claim 4, wherein the cell membranes are prepared from heterologous cells expressing said receptor.

7. The method according to any of claims 1, wherein said cells are cells isolated from tissues chosen from the group consisting of the olfactory epithelium, germ cells, testis, spleen, insulin-secreting-cells, heart, brain, trachea, intervertebral intercosa, hit joint cartilages, liver, stomach, intestinal surface, thymus, dorsal muscles and coronaries; or according to claim 4, wherein said cell membranes are prepared from said tissues.

8. The method according to claim 7, wherein said cells are neurons isolated from one of said tissues; or according to claim 4, wherein said cell membranes are prepared from said isolated neurons.

9. The method according to claim 1, wherein the membrane integrated receptor is chosen from the group consisting of a G Protein Coupled Receptor, an ion-channel, and a Tyrosine kinase receptor.

10. The method according to any of claims 1, wherein the membrane integrated receptor is chosen from the group consisting of an olfactory receptor, a pheromone receptor, a taste receptor, and, any kind of membrane-bound receptor recognizing a volatile compound.

11. The method according to any of claims 1, wherein the volatile-compound-Binding Protein (BP) is of mammalian or insect origin.

12. The method according to any of claims 1, wherein the volatile-compound-Binding Protein (BP) is chosen from the group consisting of Lipocalin, serum albumin (SA), and any protein having the capacity of binding a volatile compound and functioning as a carrier to present said volatile compound to membrane-integrated receptors.

13. The method according to any of claims 1, wherein the volatile-compound-Binding Protein is a wild type protein or a variant protein.

14. The method according to any of claims 1, wherein the volatile-compound-Binding Protein is a monomer; or present as a homodimer or heterodimer, a homomultimer or heteromultimer thereof.

15. The method according to any of claims 1, wherein the volatile-compound-Binding Protein is present in a composition.

16. The method according to claim 15, wherein said composition is a natural fluid or fractions thereof.

17. The method according to any of claims 1, wherein the volatile-compound-Binding Protein is a member of the Lipocalin family wherein said proteins have a canonical super-secondary structure.

18. The method according to any of claims 1, wherein the method is a screening method.

19. The method according to any of claims 1, wherein the method is a high throughput method.

20. A kit comprising:
a cell expressing a membrane-integrated receptor recognizing a volatile compound, or a membrane fraction thereof, and, —a volatile-compound-Binding Protein (BP), a complex thereof, or, a composition thereof.

21. Use of a kit according to claim 20 to identify and/or to confirm the binding and/or function of a volatile compound onto a membrane-integrated receptor.

22. The method according to any of claim 1, the kit according to claim 20, and the use according to claim 21, wherein the volatile-compound binding protein or the Lipocalin is chosen from the group consisting of odorant binding protein (OBP), pheromone binding protein (PBP), retinol binding protein (RBP), major urinary protein (MUP), aphrodisin and von Ebner gland protein. B
